(19) United States
(12) Patent Application Publication
(10) Pub. No.: US 2024/0279230 A1
(43) Pub. Date: Aug. 22, 2024

(54) CRYSTALLINE FORMS OF A HER2 INHIBITOR

(71) Applicant: Boehringer Ingelheim International GmbH, Ingelheim am Rhein (DE)

(72) Inventors: Ulrike WERTHMANN, Biberach an der Riss (DE); Joe Ju GAO, Southbury, CT (US); Huayu LI, Brookfield, CT (US); Jason Alan MULDER, New Milford, CT (US); Thuraya Mohamed OMAR, Bardonia, NY (US)

(21) Appl. No.: 18/544,537

(22) Filed: Dec. 19, 2023

Related U.S. Application Data
(60) Provisional application No. 63/476,733, filed on Dec. 22, 2022.
(60) Provisional application No. 63/476,715, filed on Dec. 22, 2022.

(30) Foreign Application Priority Data
Feb. 14, 2023 (EP) 23156485.7
Feb. 14, 2023 (EP) 23156619.1
Jul. 28, 2023 (EP) 23188330.7
Dec. 15, 2023 (EP) 23383302.9

(51) Int. Cl. C07D 487/04; A61K 9/10; A61K 31/519

(52) U.S. Cl. CPC C07D 487/04; A61K 9/10; A61K 31/519

(57) ABSTRACT

The present invention relates to crystalline forms of a HER2 inhibitor, processes to prepare the same, pharmaceutical compositions comprising the same and uses thereof, particularly in the treatment and/or prevention of cancer and for the preparation of a solid dispersion of the HER2 inhibitor.

### RELATED APPLICATION DISCLOSURE

This application claims the benefit of US Patent Application No. U.S. 63/476,733, filed Dec. 22, 2022; US Patent Application No. U.S. 63/476,715, filed Dec. 22, 2022; European Patent Application No. 23156485.7, filed Feb. 14, 2023; European Patent Application No. EP 23156619.1, filed Feb. 14, 2023, and European Patent Application No. EP 23188330.7, filed Jul. 28, 2023; each of which is hereby incorporated by reference herein in its entirety

### FIELD OF THE INVENTION

The present invention relates to crystalline forms of N-{1-[8-({3-methyl-4-[(1-methyl-1H-1,3-benzodiazol-5-yl)oxy]phenyl}amino)-[1,3]diazino[5,4-d]pyrimidin-2-yl]piperidin-4-yl}prop-2-enamide.

### BACKGROUND

N-{1-[8-({3-methyl-4-[(1-methyl-1H-1,3-benzodiazol-5-yl)oxy]phenyl}amino)-[1,3]-diazino[5,4-d]pyrimidin-2-yl]piperidin-4-yl}prop-2-enamide, also herein referred to as compound (1) or zongertinib, is a HER2 (ErbB2) inhibitor described in WO 2021/213800. Zongertinib is a potent and selective tyrosine kinase inhibitor of wild type and mutant HER2 that spares wild type epithelial growth factor receptor (EGFR). Therefore, it is useful for the treatment and/or prevention of diseases and/or conditions wherein the inhibition of wild type and/or mutant HER2 is of therapeutic benefit, especially oncological and/or hyperproliferative diseases, such as cancer.

Different solid state forms of an active pharmaceutical ingredient (API) often possess different properties. Differences in the physicochemical properties of solid state forms can play a crucial role for the improvement of pharmaceutical compositions, for example, pharmaceutical formulations with improved dissolution profile or with improved stability or shelf-life can become accessible due to an improved solid state form of an API. Also processing or handling of the API during the formulation process may be improved. New solid state forms of an API can thus have desirable processing properties. They can be easier to handle, better suited for storage, and/or allow for better purification, compared to other solid state forms.

For example, the tendency of an API to absorb water from the environment can negatively affect the pharmaceutical behavior and quality of a formulation comprising the API. Water absorption for example can lead to chemical degradation (e.g. via hydrolysis), trigger changes of the physical form (e.g. via hydrate formation), lead to changes in dissolution behavior and influence powder properties such as flowability, compactability, tableting, compression behavior, etc. Also, the sudden appearance or disappearance of a metastable polymorph can present a problem in pharmaceutical development. Similarly, serious consequences may arise if phase transformation occurs in a pharmaceutical dosage form, e.g. upon storage.

Therefore, there remains a need to improve the physicochemical properties e.g. the thermal stability of N-{1-[8-({3-methyl-4-[(1-methyl-1H-1,3-benzodiazol-5-yl)oxy]phenyl}amino)-[1,3]-diazino[5,4-d]pyrimidin-2-yl]piperidin-4-yl}prop-2-enamide over solutions of the prior art. A particular objective of the present invention is, for example, to improve physical and chemical stability against temperature and/or moisture stress of compound (1). Another objective of the present invention is to improve powder properties of compound (1) such as crystallinity, hygroscopicity and morphology. There is also a strong need for the provision of a form of compound (1) which exhibits low hygroscopicity and/or is thermodynamically stable to avoid phase transformation during pharmaceutical processing or storage.

### BRIEF DESCRIPTION OF THE FIGURES

FIG. 1 shows an x-ray powder diffractogram (XRPD) of crystalline form I of compound (1); the x-axis shows the scattering angle in ° 2-theta, the y-axis shows the intensity of the scattered X-ray beam in counts per second (cps) of detected photons.

FIG. 2 shows an x-ray powder diffractogram (XRPD) of crystalline form III of compound (1); the x-axis shows the scattering angle in ° 2-theta, the y-axis shows the intensity of the scattered X-ray beam in counts per second (cps) of detected photons.

FIG. 3 shows an x-ray powder diffractogram (XRPD) of crystalline form IV of compound (1); the x-axis shows the scattering angle in ° 2-theta, the y-axis shows the intensity of the scattered X-ray beam in counts per second (cps) of detected photons.

FIG. 4 shows an overlay of x-ray powder diffractograms (XRPDs) of form I (bottom), form III (middle) and form IV (top) of compound (1);

FIG. 5a shows background Raman spectra;

FIG. 5b shows a Raman spectrum of crystalline form I of compound (1);

FIG. 5c shows a Raman spectrum of crystalline form III of compound (1);

FIG. 5d shows a Raman spectrum of crystalline form IV of compound (1);

FIG. 6 shows a DSC plot of crystalline form I of compound (1);

FIG. 7 shows a DSC plot of crystalline form III of compound (1);

FIG. 8 shows a DSC plot of crystalline form IV of compound (1);

FIG. 9 shows a TGA plot of crystalline form I of compound (1);

FIG. 10 shows a TGA plot of crystalline form III of compound (1);

FIG. 11 shows a TGA plot of crystalline form IV of compound (1);

FIG. 12 shows DVS sorption and desorption isotherms of crystalline form I of compound (1);

FIG. 13 shows an overlay of x-ray powder diffractograms (XRPDs) of crystalline form I measured before (bottom) and after (top) the DVS experiment described in Example 3.5;

FIG. 14 shows DVS sorption and desorption isotherms of crystalline form III of compound (1);

FIG. 15 shows DVS sorption and desorption isotherms of crystalline form IV of compound (1);

FIG. 16 shows a SEM image of crystalline form I of compound (1) (magnification: ×2000; scale bar: 30 μm);

FIG. 17 shows a SEM image of crystalline form III of compound (1) (magnification: ×1000; scale bar: 80 μm);

FIG. 18 shows a SEM image of crystalline form IV of compound (1) (magnification: ×440; scale bar: 100 μm);

FIG. 19 shows an overlay of a form IV diffractogram calculated from SXRD data (bottom) with two experimental diffractograms of reference form IV samples (middle and top);

FIG. 20 shows an overlay of powder x-ray diffractograms of form III of compound (1) under different stress conditions: from bottom to top, reference form III sample, form III stressed at 90° C./78% RH for 21 days, form III stressed at 90° C./3% RH for 21 days, reference form III sample;

FIG. 21 shows an overlay of powder x-ray diffractograms of form IV of compound (1) under different stress conditions: from bottom to top, reference form IV sample, form IV stressed at 90° C./78% RH for 21 days, form IV stressed at 90° C./3% RH for 21 days, reference form IV sample;

FIG. 22 shows an overlay of powder x-ray diffractograms of form I of compound (1) before (bottom curve) and after (top curve) stressing at 150° C. for 6 hours.

FIG. 23 shows an overlay of powder x-ray diffractograms of form II of compound (1) before (bottom curve) and after (top curve) vacuum drying at 40° C., causing a phase change from form II to form V.

FIG. 24 shows an overlay of powder x-ray diffractograms of form VI of compound (1) before (bottom curve) and after (top curve) stressing at 120° C. for 1 hour, causing a phase change from form VI to amorphous compound (1).

FIG. 25 shows an overlay of powder x-ray diffractograms of various crystalline forms of compound (1): from bottom to top, form III, form I, form II, form V, form IV, form VI, form VII.

FIG. 26 shows the x-ray powder diffractograms (XRPDs) of spray dried amorphous solid dispersions of compound (1) with 75 wt % HPMCAS-M (top curve) and 50 wt % HPMCAS-M (bottom curve) obtained from Example 6 herein compared to the XRPD of crystalline compound (1).

FIG. 27 shows the x-ray powder diffractograms (XRPDs) of spray dried amorphous solid dispersions of compound (1) with 75 wt % PVP-VA (top curve) and 50 wt % PVP-VA (bottom curve) obtained from Example 6 herein compared to the XRPD of crystalline compound (1).

FIG. 28 shows the x-ray powder diffractograms (XRPDs) of spray dried amorphous solid dispersions of compound (1) with 75 wt % Eudragit® L100 (top curve) and 50 wt % Eudragit® L100 (bottom curve) obtained from Example 6 herein compared to the XRPD of crystalline compound (1).

FIG. 29 shows the x-ray powder diffractograms (XRPDs) of spray dried amorphous solid dispersions of compound (1) with 75 wt % HPMC HME 15LV (top curve) and 50 wt % HPMC HME 15LV (bottom curve) obtained from Example 6 herein compared to the XRPD of crystalline compound (1).

### SUMMARY

According to a first aspect, different crystalline forms of compound (1) as shown below are provided:

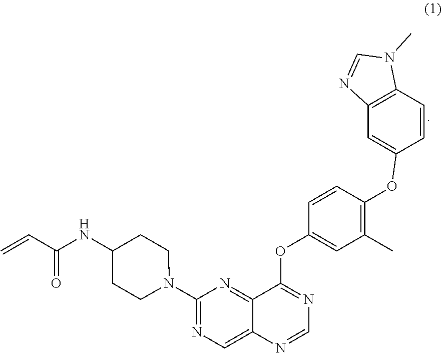 [SMILES: C=CC(=O)NC1CCN(c2ncc3ncnc(Oc4ccc(Oc5ccc6c(c5)ncn6C)c(C)c4)c3n2)CC1]

As a further aspect, methods are provided for producing crystalline forms of compound (1) as shown above. The crystalline forms of compound (1) obtained by or obtainable by such methods represent further aspects of the invention.

As a still further aspect, provided are uses and methods for treating and/or preventing an oncological and/or hyperproliferative disease, in particular cancer, with the crystalline forms of compound (1).

As yet another aspect, pharmaceutical compositions comprising the crystalline forms of compound (1) are provided.

Also provided herein are uses of said crystalline forms of compound (1) to prepare solid dispersions of compound (1). Processes of preparing solid dispersions of compound (1) with the crystalline forms of compound (1) are also provided.

### DETAILED DESCRIPTION

It is a purpose of the present invention to improve the physicochemical properties, e.g. the thermal stability of compound (1), wherein compound (1) has the following structure:

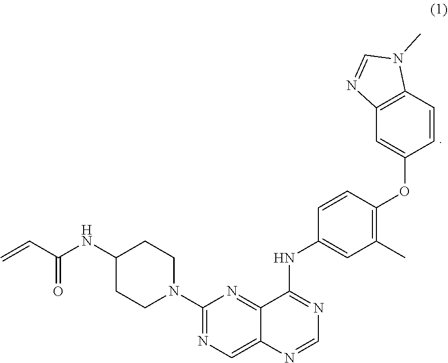 [SMILES: C=CC(=O)NC1CCN(c2ncc3ncnc(Nc4ccc(Oc5ccc6c(c5)ncn6C)c(C)c4)c3n2)CC1]

Crystalline forms I, III, and IV as defined herein possess one or more improved properties in terms of chemical stability, physical stability, melting point, hygroscopicity, morphology, solubility, crystallinity, flowability, bulk density, compactibility and wettability compared to other solid state forms. In particular, it was found that crystalline forms I, III, and IV improve physical and chemical stability against temperature and/or moisture stress of compound (1) thus allowing for its stable storage.

For example, no irreversible phase changes occurred during DVS experiments with form I, form III and form IV (see Example 3.5). In addition, form I was also polymorphically stable when subjected to temperature stress (see Example 4.2). Accelerated stress stability studies conducted on form III and form IV showed that neither form underwent phase changes even under extreme storage conditions of 90° C./3% RH and 90° C./78% RH (see Example 4.1).

Form I contains a higher level of residual solvents, including water, than forms III and IV and has a lower melting point. Form I may be converted to form III and/or form IV, e.g. under competitive slurry conditions.

Forms III and IV can allow for higher stability for storage of compound (1) than form I, which is important to maintain the quality of the compound over time.

Forms III and IV have very similar melting points. The solid-state stability data indicates they are both stable crystalline forms. Form III and IV do not readily interconvert under competitive slurry conditions.

Any of the crystalline forms I, III and IV described herein or mixtures thereof can advantageously be used to prepare a solid dispersion comprising compound (1) and a pharmaceutically acceptable dispersion carrier. Therefore, the identity of the crystalline form—whether it is form I, III or IV—is not critical to the formulation process of compound (1) to a solid dispersion. For example, forms III and IV exhibit comparative dissolution properties in solvent systems for preparing such solid dispersion.

It was discovered that crystalline forms I, III, and IV as defined herein show improved thermal stability over the amorphous form of compound (1) which directly impacts quality of the material as well as its preservation over storage. Furthermore, isolation of crystalline forms I, III, and/or IV generally is not only easier on the production scale, such as referring to solids/liquid separation, but also allows for a superior control of the chemical purity of the material relying on the well-defined crystalline lattice. It also opens an opportunity for a particle engineering via development/optimization of crystallization procedures and/or milling protocols.

Therefore, the polymorphic forms according to the present invention surprisingly show significant advantageous physicochemical properties which allow for stable manufacturing and storage of compound (1) and for reliable manufacturing of a safe and efficacious pharmaceutical drug product containing compound (1).

The expressions “crystalline form”, “polymorphic form” and “polymorph” are used interchangeably herein and refer to a crystalline solid phase chemical composition, being either a single chemical entity, e.g. compound X, or a multiple-component composition such as a salt or solvate, e.g. XₐY_{b}Z_{c}, each having a unique crystal lattice (periodic arrangement of molecules) and displaying a distinct X-ray diffraction pattern. When speaking of a crystalline form of compound (1) as shown below, any of crystalline forms I, III, and IV are meant, including the respective broader aspect or definition as well as each embodiment thereof.

The term “solid state form” as used herein refers to any crystalline and/or amorphous phase of a compound, e.g. of compound (1).

The crystalline forms of compound (1) of the present invention may be characterized by analytical methods well known in the field of the pharmaceutical industry for characterizing solids. Such methods comprise but are not limited to XRPD, ssNMR, FTIR spectroscopy, Raman spectroscopy, DSC, TGA and GMS. The crystalline forms of compound (1) of the present invention may be characterized by one of the aforementioned analytical methods or by combining two or more of them. In particular, the crystalline forms of compound (1) of the present invention may be characterized by any one of the following embodiments or by combining two or more of the following embodiments. “Cu-Kα radiation” as used in the present invention includes Cu-Kα1 radiation and Cu-Kα1,2 radiation, wherein Cu-Kα1 radiation has a wavelength of 1.54056 Å and Cu-Kα1,2 radiation has an average wavelength of 1.54184 Å. As used herein, the term “compound (1)” refers to the compound as defined below:

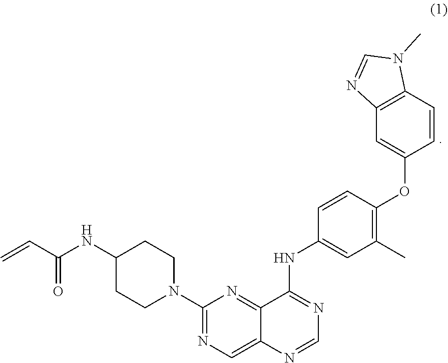 [SMILES: C=CC(=O)NC1CCN(c2ncc3ncnc(Nc4ccc(Oc5ccc6c(c5)ncn6C)c(C)c4)c3n2)CC1]

The IUPAC name of compound (1) is N-{1-[8-({3-methyl-4-[(1-methyl-1H-1,3-benzodiazol-5-yl)oxy]phenyl}amino)-[1,3]diazino[5,4-d]pyrimidin-2-yl]piperidin-4-yl}prop-2-enamide. In case of discrepancy between IUPAC name and depicted formula, the formula shall prevail. Compound (1) is disclosed in WO 2021/213800 as example compound I-01. Compound (1) is also known as zongertinib. WO 2021/213800 describes [1,3]diazino[5,4-d]pyrimidines such as compound (1) as HER2 inhibitors and provides a synthesis procedure for compound (1). Properties of compound (1) and evidence for inhibitory effect on HER2 wild-type and YVMA kinase activity, while sparing EGFR, are also disclosed in WO 2021/213800, which is herein incorporated by reference.

### Form IV

According to a first aspect, a crystalline form is provided which is also called form IV in the sense of the present invention. According to form IV of the present invention, a crystalline form of compound (1) is provided:

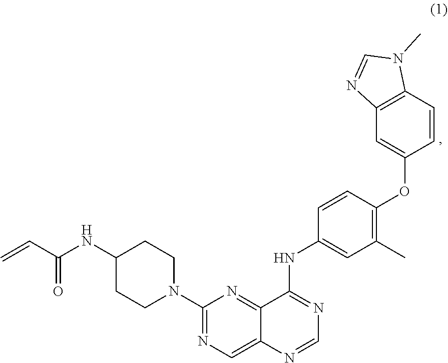 [SMILES: C=CC(=O)NC1CCN(c2ncc3ncnc(Nc4ccc(Oc5ccc6c(c5)ncn6C)c(C)c4)c3n2)CC1]

characterized by having:
(a) a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°) and (16.7±0.2°); and/or
(b) a Raman spectrum comprising a peak at any one of the following wavenumbers expressed as inverse centimeters when measured at a temperature in the range of from 20 to 30° C. and a wavelength of 785 nm: 640±2 and/or 831±2.

According to an embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°) and (16.7±0.2°).

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.1°), (11.7±0.1°) and (16.7±0.1)°.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°), (14.7±0.2°) and (16.7±0.2°).

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.1°), (11.7±0.1°), (14.7±0.1°) and (16.7±0.1°).

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°), (14.7±0.2°), (16.7±0.2°), (18.8±0.2°) and (19.2±0.2°).

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.1°), (11.7±0.1°), (14.7±0.1°), (16.7±0.1°), (18.8±0.1°) and (19.2±0.1°).

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.5±0.2°), (11.7±0.2°), (14.7±0.2°), (16.7±0.2°), (18.8±0.2°), (19.2±0.2°), (24.3±0.2°) and (25.8±0.2)°.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.1°), (11.5±0.1°), (11.7±0.1°), (14.7±0.1°), (16.7±0.1°), (18.8±0.1°), (19.2±0.1°), (24.3±0.1°) and (25.8±0.1°).

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.5±0.2°), (11.7±0.2°), (14.7±0.2°), (16.7±0.2°), (18.8±0.2°), (19.2±0.2°), (21.3±0.2°), (24.3±0.2°) and (25.8±0.2)°.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.1°), (11.5±0.1°), (11.7±0.1°), (14.7±0.1°), (16.7±0.1°), (18.8±0.1°), (19.2±0.1°), (21.3±0.1°), (24.3±0.1°) and (25.8±0.1°).

According to a further embodiment of form IV, the crystalline form has a Raman spectrum comprising a peak at any one of the following wavenumbers expressed as inverse centimeters when measured at a temperature in the range of from 20 to 30° C. and a wavelength of 785 nm: 640±2 and/or 831±2.

According to a further embodiment of form IV, the crystalline form has a Raman spectrum comprising a peak at the following wavenumber expressed as inverse centimeters when measured at a temperature in the range of from 20 to 30° C. and a wavelength of 785 nm: 640±2.

According to a further embodiment of form IV, the crystalline form has a Raman spectrum comprising a peak at the following wavenumber expressed as inverse centimeters when measured at a temperature in the range of from 20 to 30° C. and a wavelength of 785 nm: 831±2.

According to a further embodiment of form IV, the crystalline form has a Raman spectrum comprising peaks at the following wavenumbers expressed as inverse centimeters when measured at a temperature in the range of from 20 to 30° C. and a wavelength of 785 nm: 640±2 and 831±2.

According to a further embodiment of form IV, the crystalline form has a Raman spectrum comprising peaks at the following wavenumbers expressed as inverse centimeters when measured at a temperature in the range of from 20 to 30° C. and a wavelength of 785 nm: 238±2, 640±2 and 831±2.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°) and (16.7±0.2°) and a Raman spectrum comprising a peak at any one of the following wavenumbers expressed as inverse centimeters when measured at a temperature in the range of from 20 to 30° C. and a wavelength of 785 nm: 640±2 and/or 831±2.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°) and (16.7±0.2°) and a Raman spectrum comprising a peak at any one of the following wavenumbers expressed as inverse centimeters when measured at a temperature in the range of from 20 to 30° C. and a wavelength of 785 nm: 640±1 and/or 831=1.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.1°), (11.7±0.1°) and (16.7±0.1°) and a Raman spectrum comprising a peak at any one of the following wavenumbers expressed as inverse centimeters when measured at a temperature in the range of from 20 to 30° C. and a wavelength of 785 nm: 640±2 and/or 831±2.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°) and (16.7±0.2°) and a Raman spectrum comprising peaks at the following wavenumbers expressed as inverse centimeters when measured at a temperature in the range of from 20 to 30° C. and a wavelength of 785 nm: 640±2 and 831±2.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2)°, (11.7±0.2°) and (16.7±0.2°) and a Raman spectrum comprising peaks at the following wavenumbers expressed as inverse centimeters when measured at a temperature in the range of from 20 to 30° C. and a wavelength of 785 nm: 640±2 or 831±2. According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°), (14.7±0.2°), (16.7±0.2°), (18.8±0.2°) and (19.2±0.2°) and a Raman spectrum comprising peaks at the following wavenumbers expressed as inverse centimeters when measured at a temperature in the range of from 20 to 30° C. and a wavelength of 785 nm: 238±2, 640±2 and 831±2.

According to a further embodiment of form IV, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°), (14.7±0.2°), (16.7±0.2°), (18.8±0.2°) and (19.2±0.2°) and a Raman spectrum comprising peaks at the following wavenumbers expressed as inverse centimeters when measured at a temperature in the range of from 20 to 30° C. and a wavelength of 785 nm: 238±1, 640±1 and 831±1.

According to a further embodiment of form IV, the crystalline form is in substantially pure form.

The term “substantially pure” when referring to a designated crystalline form of compound (1) means that the designated crystalline form contains less than about 20% (by weight) of residual components such as alternate polymorphic or isomorphic crystalline form(s) thereof. It is preferred that a substantially pure form of compound (1) contains less than about 10% (by weight) of alternate polymorphic or isomorphic crystalline forms, more preferred is less than about 5% (by weight), such as less than about 3% (by weight), and most preferably less than about 1% (by weight) of alternate polymorphic or isomorphic crystalline forms.

The term “isomorphic forms” as used herein refers to forms having the same overall crystal structure but with minor differences in cell dimensions, i.e. showing similar but not identical XRPD patterns. Due to the relatively small size of certain solvent molecules when compared to the active compound, some solvates may be isomorphic.

In a preferred embodiment of form IV, the crystalline form has a TGA thermogram characterized by a mass loss from approximately 243° C. (+5° C.) to approximately 580° C. (±5° C.). Preferably, said mass loss is measured at a heating rate of 20° C./min. In addition or in alternative, preferably, said mass loss is of about 50 wt % based on the weight of the crystalline form.

In a preferred embodiment of form IV, the crystalline form has a TGA thermogram characterized by a mass loss from approximately 420° C. (±5° C.) to approximately 455° C. (±5° C.). Preferably, said mass loss is measured at a heating rate of 20° C./min. In addition or in alternative, preferably, said mass loss is of about 50 wt % based on the weight of the crystalline form.

As used herein, the term “approximately” and “about” mean within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range. In another embodiment of form IV, the crystalline form has a TGA thermogram showing a mass loss of not more than 2.0 wt %, preferably of not more than 1.5 wt %, even more preferably of not more than 1.3 wt %, based on the weight of the crystalline form, when heated from 25 to 244° C. at a rate of 20° C./min.

According to a further embodiment of form IV, the crystalline form has a melting point between 220° C. and 240° C. According to a further embodiment of form IV, the crystalline form has a melting point between 225° C. and 235° C. According to a further embodiment of form IV, the crystalline form has a melting point of approximately 232° C. (±5° C.). In these embodiments, the melting point is preferably determined by differential scanning calorimetry (DSC), in particular at a heating rate of 10° C./min.

In another embodiment of form IV, the crystalline form is characterized by having a DSC curve comprising an endothermic peak, preferably a single endothermic peak, having a peak onset at a temperature of (229±2° C.), preferably of (229±1° C.), for example at about 229° C., when measured at a heating rate of 10° C./min.

In a further embodiment of form IV, the crystalline form is characterized by having a DSC curve comprising an endothermic peak, preferably a single endothermic peak, having a peak maximum at a temperature of (232±2)° C., preferably of (232±1° C.), for example at about 232° C., when measured at a heating rate of 10° C./min.

In a further embodiment of form IV, the crystalline form is characterized by showing a mass change of not more than 2.0 wt %, preferably of not more than 1.0 wt %, most preferably of not more than 0.5 wt %, based on the weight of the crystalline form, when measured with DVS at a relative humidity in the range of from 0 to 90% and a temperature of (25.0±1.0° C.)

In a further embodiment of form IV, the crystalline form is characterized by showing a mass change of not more than 2.0 wt %, preferably of not more than 1.0 wt %, most preferably of not more than 0.5 wt %, such as not more than 0.4 wt % based on the weight of the crystalline form, when measured with DVS at a relative humidity in the range of from 0 to 80% and a temperature of (25.0±1.0° C.)

In one embodiment of form IV, the crystalline form is anhydrous. The terms “anhydrous” or “anhydrate” as used herein refer to a crystalline solid where no water is cooperated in or accommodated by the crystal structure. Anhydrous forms may still contain residual water, which is not part of the crystal structure but may be adsorbed on the surface or absorbed in disordered regions of the crystal. Typically, an anhydrous form does not contain more than 2.0 wt %, preferably not more than 1.0 wt % and most preferably not more than 0.5 wt % of water, based on the weight of the crystalline form.

In a further embodiment of form IV, the crystalline form is slightly hygroscopic. The term “slightly hygroscopic” as used herein refers to a compound showing a water uptake of at most 2 wt % in the sorption cycle, based on the weight of the compound, when measured with DVS at a relative humidity in the range of from 0 to 90% RH and a temperature of (25.0±1.0) ° C. In yet another embodiment of form IV, the crystalline form is characterized by exhibiting a triclinic unit cell having space group P-1. Preferably, the unit cell has approximately the following parameters
a=9.4141(12) Å
b=10.5120(13) Å
c=15.6630(19) Å
α=91.327(6)°
β=102.462(7)°
γ=114.987(8)°

when measured with single crystal x-ray diffraction at 283-303 K with CuK_{α} radiation having a wavelength of 1.54184 Å.

In a preferred embodiment, the invention relates to a composition comprising crystalline form IV as defined herein, wherein the crystalline form of compound (1) is present in an amount of at least about 50% (w/w), 60% (w/w), 65% (w/w), 67% (w/w), 70% (w/w), 75% (w/w), 80% (w/w) or 82% (w/w), preferably at least about 85% (w/w) or 88% (w/w), more preferably at least about 90% (w/w), including at least about 90, 91, 92, 93, 94, 95, 96, 97, 98 or 99% (w/w), and also including equal to about 100% (w/w), based on the weight of the composition. The remaining material may comprise other solid-state form(s) of compound (1) such as amorphous compound (1), and/or reaction impurities and/or processing impurities arising from the preparation of the composition but excluding any pharmaceutically acceptable excipients. Preferably, the remaining material is amorphous compound (1).

According to a further aspect, provided is a process for the preparation of a crystalline form of compound (1), wherein compound (1) is as follows:

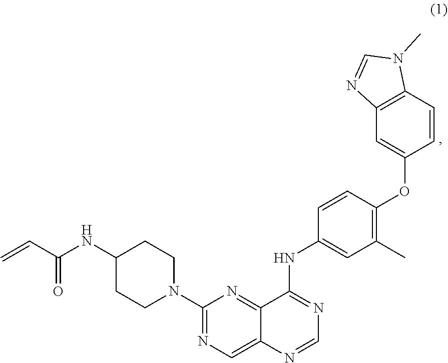 [SMILES: C=CC(=O)NC1CCN(c2ncc3ncnc(Nc4ccc(Oc5ccc6c(c5)ncn6C)c(C)c4)c3n2)CC1]

wherein the process comprises the following steps:
i) dissolving compound (1) upon heating in at least one organic solvent;
ii) cooling the solution obtained in step (i), wherein a crystalline form of compound (1) is formed; and
iii) isolating at least a part of the crystalline form of compound (1) obtained in step (ii).

Preferably, according to this process, crystalline form IV as herein defined is at least in part formed, especially selectively formed.

According to this method and according to step i), compound (1) is dissolved upon heating in at least one organic solvent. With this regard, either one or a plurality of solvents may be used. Especially, it may be preferred that the solvent is or the solvent mixture comprises an alcohol. Even more preferred, the solvent is or the solvent mixture comprises n-butanol. Dissolution may for example be reached at temperatures of equal or more than 75° C., such as at about 90° C., exemplarily by agitating the mixture.

According to step ii), the mixture is cooled with appropriate cooling rates. For example, firstly slow-cooling with a cooling rate of less than 1° C./min, such as of less than 0.5° C./min, for example of about 0.2° C./min, optionally followed by quick cooling having a higher cooling rate than the slow-cooling, such as to room temperature, for example to about 20° C. may be used. By means of cooling, crystalline form IV is formed.

Finally, the produced crystalline form may be isolated according to step iii). This may be realized by common processes. According to an embodiment, filtering and washing the crystals may be preferred.

As a further example, crystalline form IV may also be formed by in the presence of seed crystals, for example, using already formed seed crystals of crystalline form III. With this regard, in order to dissolve compound (1), a mixture of an alcohol and another organic solvent may be used. As an example for an organic solvent, anisole may be used, which may be used in an amount appropriate to dissolve compound (1). For example, a mixture of anisole with n-butanol may be used in approximately a 1:1 volume-ratio.

The alcohol may then be removed by distillation at least in part. The resulting mixture may be provided with seed crystals of crystalline form III and held at a temperature of equal or more than 100° C. The mixture may then be cooled to room temperature and the resulting solid is isolated as crystalline form IV.

According to a further aspect, provided is a crystalline form obtainable or obtained by
i) dissolving compound (1) upon heating in at least one organic solvent; and
ii) cooling the solution obtained in step (i), wherein the crystalline form of compound (1) is formed.

In embodiments of this obtained or obtainable crystalline form, steps (i) and (ii) can be performed as detailed above.

The crystalline form obtainable or obtained by the processes according to the aspects described in this section about form IV and preferred embodiments thereof is a further object of the invention. Preferably, this crystalline form is characterized by having a powder X-ray diffraction pattern comprising the peaks as defined above for form IV and/or a Raman spectrum comprising peaks at the wavenumbers as defined above for form IV. Preferably, this crystalline form corresponds to form IV as defined herein in its broadest form or in any embodiment.

### Form III

According to a further aspect, a crystalline form is provided which is also called form III in the sense of the present invention. According to form III of the present invention, a crystalline form of compound (1) is provided:

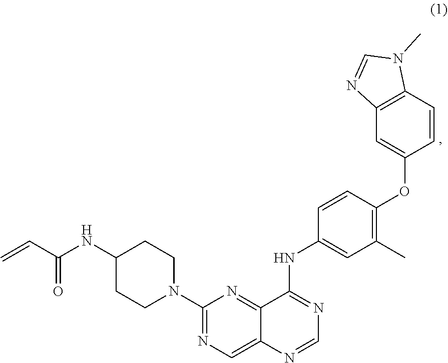 [SMILES: C=CC(=O)NC1CCN(c2ncc3ncnc(Nc4ccc(Oc5ccc6c(c5)ncn6C)c(C)c4)c3n2)CC1]

characterized by having:
(a) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°) and (16.2±0.2°); or
(b) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±
(c) a Raman spectrum comprising a peak at any one of the following wavenumbers expressed as inverse centimeters when measured at a temperature in the range of from 20 to 30° C. and a wavelength of 785 nm: 1310±2 and/or 1400±2; or 0.1°); or
(d) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°) and (16.2±0.2°) and a Raman spectrum comprising a peak at any one of the following wavenumbers expressed as inverse centimeters when measured at a temperature in the range of from 20 to 30° C. and a wavelength of 785 nm: 1310±2 and/or 1400±2; or
(e) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°) and a Raman spectrum comprising a peak at any one of the following wavenumbers expressed as inverse centimeters when measured at a temperature in the range of from 20 to 30° C. and a wavelength of 785 nm: 1310±2 and/or 1400±2.

According to an embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2)°, (11.4±0.2°), (12.4±0.2°) and (16.2±0.2°).

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°).

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°) and (16.2±0.1)°.

According to an embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (15.4±0.2°) and (16.2±0.2°)

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2=0.1°), (9.5±0.1°), (11.4±0.1°), (12.4±0.1°), (15.4±0.1°) and (16.2±0.1)°.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (16.2±0.2°) and (18.3±0.2°).

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°), (16.2±0.1°) and (18.3±0.1°).

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (13.5±0.2°), (13.8±0.2°), (16.2±0.2°) and (18.3±0.2)°

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°), (13.5±0.1°), (13.8±0.1°), (16.2±0.1°) and (18.3±0.1°).

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (15.4±0.2°), (16.2±0.2°) and (18.3±0.2°).

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°), (15.4±0.1°), (16.2±0.1)° and (18.3=0.1°).

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (13.5±0.2°), (13.8±0.2°), (14.0±0.2°), (15.4±0.2°), (16.2±0.2°) and (18.3±0.2)°.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°), (13.5±0.1°), (13.8±0.1°), (14.0±0.1°), (15.4±0.1°), (16.2±0.1°) and (18.3±0.1°).

According to a further embodiment of form III, the crystalline form has a Raman spectrum comprising a peak at any one of the following wavenumbers expressed as inverse centimeters when measured at a temperature in the range of from 20 to 30° C. and a wavelength of 785 nm: 1310±2 and/or 1400±2.

According to a further embodiment of form III, the crystalline form has a Raman spectrum comprising a peak at the following wavenumber expressed as inverse centimeters when measured at a temperature in the range of from 20 to 30° C. and a wavelength of 785 nm: 1310±2.

According to a further embodiment of form III, the crystalline form has a Raman spectrum comprising a peak at the following wavenumber expressed as inverse centimeters when measured at a temperature in the range of from 20 to 30° C. and a wavelength of 785 nm: 1400±2.

According to a further embodiment of form III, the crystalline form has a Raman comprising peaks at the following wavenumbers expressed as inverse centimeters when measured at a temperature in the range of from 20 to 30° C. and a wavelength of 785 nm: 1310±2 and 1400±2.

According to a further embodiment of form III, the crystalline form has a Raman spectrum comprising a peak at any one of the following wavenumbers expressed as inverse centimeters when measured at a temperature in the range of from 20 to 30° C. and a wavelength of 785 nm: 1310±2 and/or 1400±2 and a peak at any one of the following wavenumbers expressed as inverse centimeters when measured at a temperature in the range of from 20 to 30° C. and a wavelength of 785 nm: 485±2, 610±2 and/or 1029±2.

According to a further embodiment of form III, the crystalline form has a Raman spectrum comprising peaks at the following wavenumbers expressed as inverse centimeters when measured at a temperature in the range of from 20 to 30° C. and a wavelength of 785 nm: 485±2, 610±2, 1029±2, 1310±2 and 1400=2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°) and (16.2±0.2°) and a Raman spectrum comprising a peak at any one of the following wavenumbers expressed as inverse centimeters when measured at a temperature in the range of from 20 to 30° C. and a wavelength of 785 nm: 1310±2 and/or 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (15.4±0.2°) and (16.2±0.2°) and a Raman spectrum comprising a peak at any one of the following wavenumbers expressed as inverse centimeters when measured at a temperature in the range of from 20 to 30° C. and a wavelength of 785 nm: 1310±2 and/or 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°) and (16.2±0.2°) and a Raman spectrum comprising peaks at the following wavenumbers expressed as inverse centimeters when measured at a temperature in the range of from 20 to 30° C. and a wavelength of 785 nm: 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (15.4±0.2°) and (16.2±0.2°) and a Raman spectrum comprising peaks at the following wavenumbers expressed as inverse centimeters when measured at a temperature in the range of from 20 to 30° C. and a wavelength of 785 nm: 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°) and (16.2±0.2°) and a Raman spectrum comprising peaks at the following wavenumbers expressed as inverse centimeters when measured at a temperature in the range of from 20 to 30° C. and a wavelength of 785 nm: 485±2, 610±2, 1029±2, 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (15.4±0.2°) and (16.2±0.2°) and a Raman spectrum comprising peaks at the following wavenumbers expressed as inverse centimeters when measured at a temperature in the range of from 20 to 30° C. and a wavelength of 785 nm: 485±2, 610±2, 1029±2, 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1)°, (9.5±0.1°), (11.4±0.1°) and (12.4±0.1°) and a Raman spectrum comprising a peak at any one of the following wavenumbers expressed as inverse centimeters when measured at a temperature in the range of from 20 to 30° C. and a wavelength of 785 nm: 1310±2 and/or 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°) and a Raman spectrum comprising peaks at the following wavenumbers expressed as inverse centimeters when measured at a temperature in the range of from 20 to 30° C. and a wavelength of 785 nm: 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°) and (16.2±0.1°) and a Raman spectrum comprising a peak at any one of the following wavenumbers expressed as inverse centimeters when measured at a temperature in the range of from 20 to 30° C. and a wavelength of 785 nm: 1310±2 and/or 1400=2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°), (15.4±0.1°) and (16.2±0.1)° and a Raman spectrum comprising a peak at any one of the following wavenumbers expressed as inverse centimeters when measured at a temperature in the range of from 20 to 30° C. and a wavelength of 785 nm: 1310±2 and/or 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°), (12.4±0.1°) and (16.2±0.1°) and a Raman spectrum comprising peaks at the following wavenumbers expressed as inverse centimeters when measured at a temperature in the range of from 20 to 30° C. and a wavelength of 785 nm: 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1)°, (11.4±0.1°), (12.4±0.1°), (15.4±0.1°) and (16.2±0.1)° and a Raman spectrum comprising a peak at any one of the following wavenumbers expressed as inverse centimeters when measured at a temperature in the range of from 20 to 30° C. and a wavelength of 785 nm: 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°) and a Raman spectrum comprising peaks at the following wavenumbers expressed as inverse centimeters when measured at a temperature in the range of from 20 to 30° C. and a wavelength of 785 nm: 485±2, 610±2, 1029±2, 1310±2 and 1400±2.

According to a further embodiment of form III, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (15.4±0.2°), (16.2±0.2°) and (18.3±0.2°) and a Raman spectrum comprising peaks at the following wavenumbers expressed as inverse centimeters when measured at a temperature in the range of from 20 to 30° C. and a wavelength of 785 nm: 485±2, 610±2, 1029±2, 1310±2 and 1400±2.

According to a further embodiment of form III, the crystalline form is in substantially pure form.

In a preferred embodiment of form III, the crystalline form has a TGA thermogram characterized by a mass loss from approximately 240° C. (+5° C.) to approximately 580° C. (+5° C.). Preferably, said mass loss is measured at a heating rate of 20° C./min. In addition or in alternative, preferably, said mass loss is of approximately 61 wt %, based on the weight of the crystalline form.

In a preferred embodiment of form III, the crystalline form has a TGA thermogram characterized by a mass loss from approximately 420° C. (+5° C.) to approximately 454° C. (+5° C.). Preferably, said mass loss is measured at a heating rate of 20° C./min. In addition or in alternative, preferably, said mass loss is of approximately 61 wt %, based on the weight of the crystalline form.

In another embodiment of form III, the crystalline form has a TGA thermogram showing a mass loss of not more than 2.0 wt %, preferably of not more than 1.5 wt %, even more preferably of not more than 1.1 wt %, based on the weight of the crystalline form, when heated from 25 to 240° C. at a rate of 20° C./min.

According to a further embodiment of form III, the crystalline form has a melting point between 220° C. and 240° C. According to a further embodiment of form III, the crystalline form has a melting point between 225° C. and 230° C. According to a further embodiment of form III, the crystalline form has a melting point of approximately 231 (+5° C.). In these embodiments, the melting point is preferably determined by differential scanning calorimetry (DSC), in particular at a heating rate of 10° C./min.

In another embodiment of form III, the crystalline form is characterized by having a DSC curve comprising an endothermic peak, preferably a single endothermic peak, having a peak onset at a temperature of (228±2)° C., preferably of (228±1)° C., for example at about 228° C., when measured at a heating rate of 10° C./min.

In a further embodiment of form III, the crystalline form is characterized by having a DSC curve comprising an endothermic peak, preferably a single endothermic peak, having a peak maximum at a temperature of (231±2)° C., preferably of (231±1° C.), for example at about 231ºC, when measured at a heating rate of 10° C./min.

In a further embodiment of form III, the crystalline form is characterized by showing a mass change of not more than 2.0 wt %, preferably of not more than 1.7 wt %, based on the weight of the crystalline form, when measured with DVS at a relative humidity in the range of from 0 to 90% and a temperature of (25.0±1.0)° C.

In a further embodiment of form III, the crystalline form is characterized by showing a mass change of not more than 2.0 wt %, preferably of not more than 1.5 wt %, most preferably of not more than 1.2 wt %, based on the weight of the crystalline form, when measured with DVS at a relative humidity in the range of from 0 to 80% and a temperature of (25.0±1.0° C.)

In one embodiment of form III, the crystalline form is anhydrous.

In afurther embodiment of form III, the crystalline form is slightly hygroscopic.

In a preferred embodiment, the invention relates to a composition comprising crystalline form III as defined herein, wherein the crystalline form of compound (1) is present in an amount of at least about 50% (w/w), 60% (w/w), 65% (w/w), 67% (w/w), 70% (w/w), 75% (w/w), 80% (w/w) or 82% (w/w), preferably at least about 85% (w/w) or 88% (w/w), more preferably at least about 90% (w/w), including at least about 90, 91, 92, 93, 94, 95, 96, 97, 98 or 99% (w/w), and also including equal to about 100% (w/w), based on the weight of the composition. The remaining material may comprise other solid-state form(s) of compound (1) such as amorphous compound (1), and/or reaction impurities and/or processing impurities arising from the preparation of the composition but excluding any pharmaceutically acceptable excipients. Preferably, the remaining material is amorphous compound (1).

According to a further aspect, provided is a process for the preparation of a crystalline form of compound (1), wherein compound (1) is as follows:

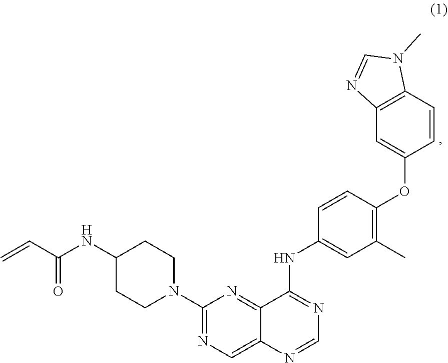 [SMILES: C=CC(=O)NC1CCN(c2ncc3ncnc(Nc4ccc(Oc5ccc6c(c5)ncn6C)c(C)c4)c3n2)CC1]

wherein the process comprises the following steps:
i) providing a suspension of compound (1) in at least one organic solvent;
ii) agitating the suspension obtained in step (i) until a crystalline form of compound (1) is formed; and
iii) isolating at least a part of the crystalline form of compound (1) obtained in step (ii).

Preferably, according to this process, crystalline form III as herein defined is at least in part formed, especially selectively formed.

According to this method and according to step i), compound (1) is suspended or slurried preferably upon heating in at least one organic solvent. With this regard, either one or a plurality of organic solvents may be used. In a preferred embodiment of this aspect, the at least one organic solvent is an alcohol or an ester. In a preferred embodiment of this aspect, the at least one organic solvent is an ester. In a preferred embodiment of this aspect, the at least one organic solvent is isopropanol or isopropyl acetate (IPAc). In a preferred embodiment of this aspect, the at least one organic solvent is isopropyl acetate (IPAc). Suspending may for example be reached at temperatures of equal or more than 50° C., such as at approximately 70° C. (+5° C.), exemplarily by agitating the mixture.

Agitating is performed according to step ii). In an embodiment of this aspect, the agitating of step (ii) is at a temperature of 18° C. to 75° C. and/or for 1-18 hours. In an embodiment of this aspect, the agitating of step (ii) is at a temperature of at least 50° C. and/or for at least 8 hours. In an alternative or additional preferred embodiment of this aspect, the agitating of step (ii) is at a temperature of 55° C. to 75° C. and/or for 10 to 18 hours. In an alternative embodiment of this aspect, the agitating of step (ii) is at a temperature of 18° C. to 30° C. and/or for 1 to 3 hours, for example for approximately 2 hours.

During agitation, the slurry or suspension may be cooled with appropriate cooling rates. Preferred cooling rates may decrease in subsequent cooling phases. In general, cooling from before-described agitating temperature to room temperature may take place. During cooling, crystalline form III can be formed as a solid.

The so-formed solid may then be isolated. This may be realized by common processes. According to an embodiment, isolating may take place by means of filtering the mixture. According to an embodiment, before-described process may comprise an additional step of seeding. With this regard, seed crystals of crystalline form III may be added to the suspension preferably at the beginning of the agitating step ii).

According to a further aspect, provided is a crystalline form obtained or obtainable by
i) providing a suspension of compound (1) in at least one organic solvent; and
ii) agitating the suspension obtained in step (i) until a crystalline form of compound (1) is formed.

In embodiments of this obtained or obtainable crystalline form, steps (i) and (ii) can be performed as detailed above.

The crystalline form obtainable or obtained by the processes according to the aspects described in this section about form III and preferred embodiments thereof is a further object of the invention. Preferably, this crystalline form is characterized by having a powder X-ray diffraction pattern comprising the peaks as defined above for form III and/or a Raman spectrum comprising peaks at the wavenumbers as defined above for form III. Preferably, this crystalline form corresponds to form III as defined herein in its broadest form or in any embodiment.

### Form I

According to a further aspect, a crystalline form is provided which is also called form I in the sense of the present invention. According to form I of the present invention, a crystalline form of compound (1) is provided

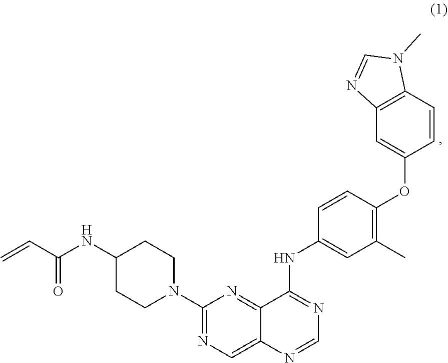 [SMILES: C=CC(=O)NC1CCN(c2ncc3ncnc(Nc4ccc(Oc5ccc6c(c5)ncn6C)c(C)c4)c3n2)CC1]

characterized by having:
(a) a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.1±0.2°); and/or
(b) a Raman spectrum comprising a peak at any one of the following wavenumbers expressed as inverse centimeters when measured at a temperature in the range of from 20 to 30° C. and a wavelength of 785 nm: 1411±2 and/or 1602±2.

Alternatively, according to form I of the present invention, a crystalline form of compound (1) is provided

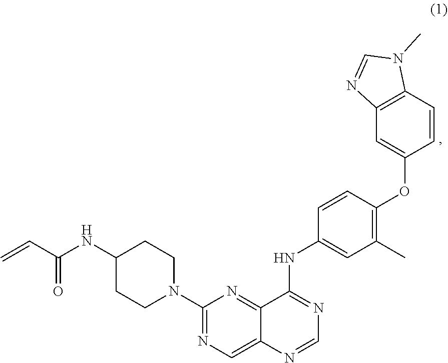 [SMILES: C=CC(=O)NC1CCN(c2ncc3ncnc(Nc4ccc(Oc5ccc6c(c5)ncn6C)c(C)c4)c3n2)CC1]

characterized by having:
(a) a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2)° and (12.0±0.2)°; and/or
(b) a Raman spectrum comprising a peak at any one of the following wavenumbers expressed as inverse centimeters when measured at a temperature in the range of from 20 to 30° C. and a wavelength of 785 nm: 1411±2 and/or 1602±2.

According to an embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.1±0.2)°.

According to an embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.0±0.2)°.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.1)° and (12.1±0.1)°.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.1)° and (12.0±0.1)°.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.2°), (7.9±0.2°), (11.1±0.2°), (12.0±0.2°), (17.2±0.2°) and (17.9±0.2°).

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.1°), (7.9±0.1°), (11.1±0.1°), (12.0±0.1°), (17.2±0.1°) and (17.9±0.1)°.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.2°), (7.9±0.2°), (11.1±0.2°), (12.0±0.2°), (13.9±0.2°), (15.6±0.2°), (17.2±0.2°), (17.9±0.2°) and (18.8±0.2)°.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.1°), (7.9±0.1°), (11.1±0.1°), (12.0±0.1°), (13.9±0.1°), (15.6±0.1°), (17.2±0.1°), (17.9±0.1°) and (18.8±0.1°).

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.2°), (7.9±0.2°), (11.1±0.2°), (12.0±0.2°), (12.9±0.2°), (13.9±0.2°), (15.6±0.2°), (17.2±0.2°), (17.9±0.2°) and (18.8±0.2°).

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.1°), (7.9±0.1°), (11.1±0.1°), (12.0±0.1°), (12.9±0.1°), (13.9±0.1°), (15.6±0.1°), (17.2±0.1°), (17.9±0.1°) and (18.8±0.1°).

According to a further embodiment of form I, the crystalline form has a Raman spectrum comprising a peak at any one of the following wavenumbers expressed as inverse centimeters when measured at a temperature in the range of from 20 to 30° C. and a wavelength of 785 nm: 1411±2 and/or 1602±2.

According to a further embodiment of form I, the crystalline form has a Raman spectrum comprising a peak at the following wavenumber expressed as inverse centimeters when measured at a temperature in the range of from 20 to 30° C. and a wavelength of 785 nm: 1411±2.

According to a further embodiment of form I, the crystalline form has a Raman spectrum comprising a peak at the following wavenumber expressed as inverse centimeters when measured at a temperature in the range of from 20 to 30° C. and a wavelength of 785 nm: 1602±2.

According to a further embodiment of form I, the crystalline form has a Raman spectrum comprising peaks at the following wavenumbers expressed as inverse centimeters when measured at a temperature in the range of from 20 to 30° C. and a wavelength of 785 nm: 1411±2 and 1602±2.

According to a further embodiment of form I, the crystalline form has a Raman spectrum comprising peaks at the following wavenumbers expressed as inverse centimeters when measured at a temperature in the range of from 20 to 30° C. and a wavelength of 785 nm: 824±2, 1411±2 and 1602±2.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.1±0.2°) and a Raman spectrum comprising a peak at any one of the following wavenumbers expressed as inverse centimeters when measured at a temperature in the range of from 20 to 30° C. and a wavelength of 785 nm: 1411±2 and/or 1602±2.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.0±0.2°) and a Raman spectrum comprising a peak at any one of the following wavenumbers expressed as inverse centimeters when measured at a temperature in the range of from 20 to 30° C. and a wavelength of 785 nm: 1411±2 and/or 1602±2.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.1°) and (12.1±0.1°) and a Raman spectrum comprising a peak at any one of the following wavenumbers expressed as inverse centimeters when measured at a temperature in the range of from 20 to 30° C. and a wavelength of 785 nm: 1411±2 and/or 1602±2.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.1°) and (12.0±0.1°) and a Raman spectrum comprising a peak at any one of the following wavenumbers expressed as inverse centimeters when measured at a temperature in the range of from 20 to 30° C. and a wavelength of 785 nm: 1411±2 and/or 1602±2.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.1±0.2°) and a Raman spectrum comprising peaks at the following wavenumbers expressed as inverse centimeters when measured at a temperature in the range of from 20 to 30° C. and a wavelength of 785 nm: 1411±2 and 1602±2.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.0±0.2°) and a Raman spectrum comprising peaks at the following wavenumbers expressed as inverse centimeters when measured at a temperature in the range of from 20 to 30° C. and a wavelength of 785 nm: 1411±2 and 1602±2.

According to a further embodiment of form I, the crystalline form has a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.2°), (7.9±0.2°), (11.1±0.2°), (12.0±0.2°), (17.2±0.2°) and (17.9±0.2°) and a Raman spectrum comprising peaks at the following wavenumbers expressed as inverse centimeters when measured at a temperature in the range of from 20 to 30° C. and a wavelength of 785 nm: 824±2, 1411±2 and 1602±2.

According to a further embodiment of form I, the crystalline form is in substantially pure form. In a preferred embodiment of form I, the crystalline form has a TGA thermogram characterized by a mass loss from approximately 100° C. (+5° C.) to approximately 580° C. (+5° C.).

Preferably, said mass loss is measured at a heating rate of 20° C./min. In addition or in alternative, preferably, said mass loss is of approximately 48 wt % based on the weight of the crystalline form.

In a preferred embodiment of form I, the crystalline form has a TGA thermogram characterized by a mass loss from approximately 419° C. (+5° C.) to approximately 463° C. (+5° C.).

Preferably, said mass loss is measured at a heating rate of 20° C./min. In addition or in alternative, preferably, said mass loss is of approximately 48 wt % based on the weight of the crystalline form.

According to a further embodiment of form I, the crystalline form has a melting point between 160° C. and 180 ºC. According to a further embodiment of form I, the crystalline form has a melting point between 165° C. and 175° C. According to a further embodiment of form I, the crystalline form has a melting point of approximately 171° C. (+5° C.). In these embodiments, the melting point is preferably determined by differential scanning calorimetry (DSC), in particular at a heating rate of 10° C./min.

In another embodiment of form I, the crystalline form is characterized by having a DSC curve comprising an endothermic peak having a peak onset at a temperature of (165±2° C.), preferably of (165±1° C.), for example at about 165° C., when measured at a heating rate of 10° C./min. In a further embodiment of form I, the crystalline form is characterized by having a DSC curve comprising an endothermic peak having a peak maximum at a temperature of (171±2° C.), preferably of (171±1)° C., for example at about 171ºC, when measured at a heating rate of 10° C./min.

In a preferred embodiment, the invention relates to a composition comprising crystalline form I as defined herein, wherein the crystalline form of compound (1) is present in an amount of at least about 50% (w/w), 60% (w/w), 65% (w/w), 67% (w/w), 70% (w/w), 75% (w/w), 80% (w/w) or 82% (w/w), preferably at least about 85% (w/w) or 88% (w/w), more preferably at least about 90% (w/w), including at least about 90, 91, 92, 93, 94, 95, 96, 97, 98 or 99% (w/w), and also including equal to about 100% (w/w), based on the weight of the composition. The remaining material may comprise other solid-state form(s) of compound (1) such as amorphous compound (1), and/or reaction impurities and/or processing impurities arising from the preparation of the composition but excluding any pharmaceutically acceptable excipients. Preferably, the remaining material is amorphous compound (1).

According to a further aspect, provided is a process for the preparation of a crystalline form of compound (1), wherein compound (1) is as follows:

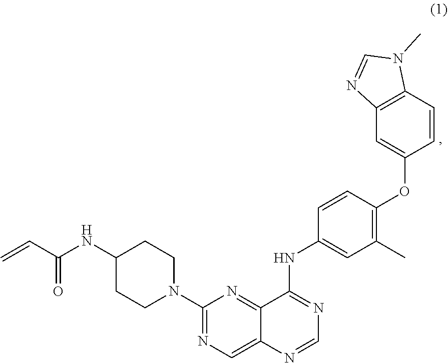 [SMILES: C=CC(=O)NC1CCN(c2ncc3ncnc(Nc4ccc(Oc5ccc6c(c5)ncn6C)c(C)c4)c3n2)CC1]

wherein the process comprises the following steps:
i) dissolving compound (1) upon heating in a mixture of solvents comprising at least one water-miscible organic solvent and water;
ii) cooling the solution obtained in step (i), wherein a crystalline form of compound (1) is formed; and
iii) isolating at least a part of the crystalline form of compound (1) obtained in step (ii).

Preferably, according to this process, crystalline form I as herein defined is at least in part formed, especially selectively formed.

According to this method and according to step i), compound (1) is dissolved upon heating in at least one water-miscible organic solvent and water. Especially, it may be preferred that the water-miscible organic solvent comprises an alcohol. Even more preferred, the water-miscible organic solvent comprises isopropyl alcohol. The amount of water-miscible organic solvent may be in the range of equal or less than 10% w/w H₂O. Dissolution may for example be reached at temperatures of equal or more than 75° C., such as at about 90° C., exemplarily by agitating the mixture.

According to step ii), the mixture is cooled to appropriate temperatures, such as equal or below 85° C., exemplarily to approximately 75° C., and the solution is agitated. Optional seeding with crystalline form I may take place. By means of cooling preferably to room temperature, such as to about 20° C., crystalline form I can be formed.

Finally, the produced crystalline form may be isolated according to step iii). This may be realized by common processes. According to an embodiment, filtering and washing the crystals may be preferred.

As a further example, crystalline form I may also be formed by dissolving compound (1) in a mixture of organic solvents comprising an alcohol and further one or more organic solvents. As an example, a mixture of dichloromethane, tetrahydrofuran and methanol may be used. The mixture may be washed, such as with brine. The mixture may then be distilled and diluted with an organic solvent, such as tetrahydrofuran. Distillation and dilution may be repeated until the amount of water and alcohol is equal or below 1.0% w/w each. Upon cooling and holding the mixture at room temperature, such as at 20° C., crystalline form I is formed. This may be isolated, such as by filtration.

According to another aspect, crystalline form I is obtained as described in Steps 1 to 6 of Example 1.1 (presented below).

According to another aspect, crystalline form I is obtained as described in Steps 1 to 5 of Example 1.2 (presented below).

According to a further aspect, provided is a crystalline form obtained or obtainable by
i) dissolving compound (1) upon heating in a mixture of solvents comprising at least one water-miscible organic solvent and water; and
ii) cooling the solution obtained in step (i), wherein a crystalline form of compound (1) is formed.

In embodiments of this obtained or obtainable crystalline form, steps (i) and (ii) can be performed as detailed above.

The crystalline form obtainable or obtained by the processes according to the aspects described in this section about form I and preferred embodiments thereof is a further object of the invention. Preferably, this crystalline form is characterized by having a powder X-ray diffraction pattern comprising the peaks as defined above for form I and/or a Raman spectrum a Raman spectrum comprising peaks at the wavenumbers as defined above for form I. Preferably, this crystalline form corresponds to form I as defined herein in its broadest form or in any embodiment.

### Solid Dispersion

All solid state forms as described herein, individually or as a mixture, can be useful to generate solid dispersions. Therefore, further provided herein is the use of any one of crystalline forms I, III, IV as described above in the broadest form or in any embodiment to produce a solid dispersion comprising compound (1) and a pharmaceutically acceptable dispersion carrier. Also provided herein is the use of any one of crystalline forms I, III, IV (as described above in the broadest form or in any embodiment) or a mixture thereof to produce a solid dispersion comprising compound (1) and a pharmaceutically acceptable dispersion carrier. In such solid dispersion, compound (1) is preferably amorphous. In other words, the solid dispersion preferably comprises compound (1) in amorphous form.

Also provided herein is the use of any one of crystalline forms I, III, IV (as described above in the broadest form or in any embodiment) or a mixture thereof to produce a solid dispersion consisting essentially of compound (1) and a pharmaceutically acceptable dispersion carrier. As used herein, the expressions “consists essentially of” and “consisting essentially of” have the meaning attributed to them in the art. In particular, they indicate that further components may be present, especially those further components that do not have a material effect on the characteristics of the respective dispersion, composition or formulation. Such further components may for example be residual solvents.

Also provided herein is the use of any one of crystalline forms I, III, IV (as described above in the broadest form or in any embodiment) or a mixture thereof to produce a solid dispersion consisting of compound (1) and a pharmaceutically acceptable dispersion carrier.

As used herein, the term “solid dispersion” refers to a system in a solid state comprising at least two components, wherein one component, such as compound (1) or generally an active pharmaceutical ingredient (API), preferably in amorphous state, is dispersed throughout another component such as a pharmaceutically acceptable solid dispersion carrier, particularly a dispersion polymer.

The phrase “pharmaceutically acceptable” is employed herein to refer to those compounds, materials, compositions, and/or dosage forms which are, within the scope of sound medical judgement, suitable for use in contact with the tissues of human beings without excessive toxicity, irritation, allergic response, or other problem or complication, and commensurate with a reasonable benefit/risk ratio.

As used herein, the term “dispersion carrier” refers to a carrier component that allows for an API such as compound (1) to be dispersed throughout such that a solid dispersion may form. In embodiments, compound (1) is dispersed at the molecular level in the pharmaceutically acceptable dispersion carrier

In embodiments, the pharmaceutically acceptable dispersion carrier is a polymer. Therefore, in the uses and processes described herein, the solid dispersion preferably comprises compound (1) as defined herein or a pharmaceutically acceptable salt thereof and a polymer. Polymeric dispersion carriers also are denoted “dispersion polymers”. Polymers are widely used in solid dispersion formulations. Different polymeric carriers lead to solid dispersions with various properties in terms of physical stability, phase behavior and drug release rate and extent. Due to the complex nature solid dispersion formulation carrier best suited for a given API need to be tested. The pharmaceutically acceptable dispersion polymer preferably is a neutral or acidic polymer.

In other embodiments, the pharmaceutically acceptable dispersion carrier is a polymer that is enteric (acidic polymer) or non-enteric (neutral polymer), preferably enteric. In other embodiments, the polymer is enteric or non-enteric, preferably enteric. The term “enteric polymer” refers to a pH-dependent acidic polymer that is insoluble or only slightly soluble at a low pH (e.g. about pH 1 up to but less than pH 3) but becomes soluble at a higher pH (e g. pH 5 and above). In certain embodiments a pH-dependent polymer may become soluble at a pH range from about pH 5 and above, e g. from about pH 6 to about pH 9, from about pH 6 to about pH 8, from about pH 5 to about pH 7, or from about pH 5 to about pH 6, which is generally less acidic than the gastric environment and roughly corresponds to pH values in the small intestine. Examples of enteric polymers include but are not limited to methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate, HPMCAS), polyvinyl acetate phthalate (PVAP), methyl methacrylate-methacrylic acid copolymers (Eudragit® L100), shellac, cellulose acetate trimellitate, sodium alginate and zein. The term “non-enteric polymer” refers to a neutral polymer that does not show pH-dependent solubility characteristics. Examples of non-enteric polymers include but are not limited to cellulose derivatives such as Methylcellulose (MC), ethylcellulose (EC), hydroxypropylcellulose (HPC), hydroxyethyl cellulose (HEC), hydroxypropyl methylcellulose (HPMC), poly-vinyl-pyrrolidone (PVP), copovidone such as polyvinylpyrrolidone-vinyl acetate copolymer (PVP-VA), poly (ethylene glycol) PEGs, starch derivatives like cyclodextrin, Soluplus® which is an amphiphilic copolymer consisting of polyethylene glycol, polyvinyl caprolactam, and polyvinyl acetate

In embodiments, the pharmaceutically acceptable dispersion carrier is a polymer, or more simply the polymer is, selected from the group consisting of hydroxypropyl methylcelluloses and esters thereof, polyvinylpyrrolidones and copolymers thereof, and polymethacrylates and copolymers thereof. The pharmaceutically acceptable dispersion carrier may contain a mixture of two or more polymers.

In an embodiment, the hydroxypropyl methylcelluloses and esters thereof are selected from the group consisting of hydroxypropyl methyl cellulose acetate (HPMCA), hydroxypropyl methyl cellulose (HPMC), hydroxypropyl cellulose (HPC), methyl cellulose, hydroxyethyl methyl cellulose, hydroxyethyl cellulose, hydroxyethyl cellulose acetate, hydroxyethyl ethyl cellulose, hydroxypropyl methyl cellulose acetate succinate (HPMCAS), hydroxypropyl methyl cellulose phthalate (HPMCP), carboxymethyl ethyl cellulose (CMEC), cellulose acetate phthalate (CAP), cellulose acetate succinate (CAS), hydroxypropyl methyl cellulose acetate phthalate (HPMCAP), cellulose acetate trimellitate (CAT), hydroxypropyl methyl cellulose acetate trimellitate (HPMCAT), and carboxymethylcellulose acetate butyrate (CMCAB). In an embodiment, the hydroxypropyl methylcelluloses and esters thereof are selected from the group consisting of hydroxypropyl methylcellulose acetate succinate and hydroxypropyl methylcellulose, in particular hot melt extrusion-grade hydroxypropyl methylcellulose.

In an embodiment, the polyvinylpyrrolidones and copolymers thereof are selected from the group consisting of polyvinylpyrrolidone vinyl acetate copolymer (PVP-VA), polyvinyl alcohols, polyvinyl alcohol polyvinyl acetate copolymers and polyvinylpyrrolidone (PVP). Polyvinylpyrrolidone (PVP) also is commonly denoted polyvidone or povidone. In embodiments, the polyvinylpyrrolidones and copolymers thereof are a polyvinylpyrrolidone vinyl acetate copolymer (PVP-VA).

In an embodiment, the polymethacrylates and copolymers thereof are selected from the group consisting of methacrylic acid-ethyl acrylate copolymer, methacrylic acid-methyl methacrylate copolymer, methyl methacrylate and methacrylic acid copolymer. Polymethacrylates and copolymers thereof are, for example, available under the brand name EudragitR from Evonik Industries AG. Methacrylic acid-methyl methacrylate copolymer is, for example, available under the brand name EudragitR L100. In certain embodiments, the polymethacrylates and copolymers thereof are a methylacrylic acid methyl methacrylate copolymer.

In embodiments, the pharmaceutically acceptable dispersion carrier is a polymer, or more simply the polymer is, selected from the group of hydroxypropyl methylcellulose acetate succinate (HPMCAS), polyvinylpyrrolidone vinyl acetate copolymer (PVP-VA), methylacrylic acid methyl methacrylate copolymer (such as EudragitR L100), and hot melt extrusion-grade hydroxypropyl methylcellulose (HPMC HME).

In certain embodiments, the pharmaceutically acceptable dispersion carrier is hydroxypropyl methylcellulose acetate succinate (HPMCAS). HPMCAS also is known as hypromellose acetate succinate. Hypromellose acetate succinate (HPMCAS) can be obtained by introducing acetyl and succinoyl groups to the hydroxyl groups of the backbone of hydroxypropyl methylcellulose (HPMC) also known as hypromellose. This procedure can be carried out by known methods, for instance by treating HPMC with acetic anhydride and/or with succinic anhydride. Acetic anhydride and succinic anhydride can be reacted with hydroxypropyl methylcellulose (HPMC) under specifically controlled conditions to produce HPMCAS with varying extent of substitution of acetyl and succinoyl groups.

HPMCAS is available in several grades (L, M and H) varying in extent of substitution of acetyl and succinoyl groups, based on the content of acetyl and succinoyl groups (wt %) in the HPMCAS molecule. Any grade of HPMCAS is usable in the solid dispersion of the invention. Preferably, HPMCAS of grade L, M or H is used. In certain embodiments, the pharmaceutically acceptable dispersion carrier is HPMCAS grade L. In certain embodiments, the pharmaceutically acceptable dispersion carrier is HPMCAS grade M. HPMCAS grade M may comprise an acetyl content of 7-11 wt %; a succinoyl content of 10-14 wt %; methoxyl content of 21-25 wt %; and a hydroxypropoxy content of 5-9 wt %. Preferably, HPMCAS grade M (HPMCAS-M) is soluble at pH ≥6. In certain embodiments, the pharmaceutically acceptable dispersion carrier is HPMCAS grade H. Preferably, granular HPMCAS (HPMCAS-G) is used. HPMCAS-G can be used for any grade of HPMCAS, in particular for grade G, such that HPMCAS-MG is used.

In certain embodiments, the pharmaceutically acceptable dispersion carrier is polyvinylpyrrolidone vinyl acetate copolymer (PVP-VA). Polyvinylpyrrolidone vinyl acetate copolymers are linear, random copolymers that are available by free-radical polymerization of the monomers in ratios varying from 70/30 to 30/70 vinyl acetate to vinylpyrrolidone.

In certain embodiments, the pharmaceutically acceptable dispersion carrier is methylacrylic acid methyl methacrylate copolymer, such as Eudragit® L100. As used herein, “methylacrylic acid methyl methacrylate copolymer” is used interchangeably with “methacrylic acid methyl methacrylate copolymer”.

In certain embodiments, the pharmaceutically acceptable dispersion carrier is a hot melt extrusion-grade hydroxypropyl methylcellulose (HPMC HME). HPMC HME refers to a modified grade of hydroxypropyl methylcellulose having low glass transition temperature and melt viscosity, which can be used for making a solid dispersion via hot melt extrusion. HPMC HME is a water-soluble amorphous polymer, usually provided as a white to off-white powder, available in three grades, HPMC HME 15 LV, HPMC HME 100 LV and HPMC HME 4M, differing in regard to their molecular weight. Preferably, HPMC HME 15LV having a molecular weight (Mw) below 100 kDa is used. Further preferred, HPMC HME 100L V having a molecular weight (Mw) below 200 kDa is used.

By dispersing the crystalline forms of compound (1), preferably on a molecular level, in a, for example polymeric, pharmaceutically acceptable dispersion carrier, an amorphous state of compound (1) can be obtained and maintained, even when exposed to elevated temperature and/or humidity conditions, and the solid dispersion can reliably provide compound (1) in amorphous form In embodiments of the solid dispersion, compound (1) is amorphous.

The term “amorphous” as used herein refers to a condensed phase where molecules are randomly orientated and characterized by the absence of any microscopic order, with no diffraction peaks by XRPD; an amorphous solid system may be composed of a single chemical entity or may be a multi-component system containing, e.g., an API, polymer and other excipients, without stoichiometric composition. Amorphous solids generally possess crystal-like short range molecular arrangement, but no long-range order of molecular packing as found in crystalline solids. The solid state form of a solid may be determined e.g. by x-ray powder diffraction (“XRPD”) or modulated differential scanning calorimetry (“mDSC”).

In embodiments, the solid dispersion comprises, consists of or consists essentially of amorphous compound (1) and a pharmaceutically acceptable dispersion carrier, wherein compound (1) is substantially in amorphous solid state form. In certain embodiments, the substantially amorphous solid state form refers to the solid dispersion comprising at least 80 wt % amorphous compound (1) based on a total weight of 100 wt % of compound (1). In certain embodiments, the substantially amorphous solid state form refers to the solid dispersion comprising at least 85 wt % amorphous compound (1) based on a total weight of 100 wt % of compound (1). In certain embodiments, the substantially amorphous solid state form refers to the solid dispersion comprising at least 90 wt % amorphous compound (1) based on a total weight of 100 wt % of compound (1). In certain embodiments, the substantially amorphous solid state form refers to the solid dispersion comprising at least 95 wt % amorphous compound (1) based on a total weight of 100 wt % of compound (1). In certain embodiments, the substantially amorphous solid-state form refers to the solid dispersion comprising at least 96, 97, 98 or 99 wt % amorphous compound (1) based on a total weight of 100 wt % of compound (1). Thus, the solid dispersion can provide compound (1) in amorphous or essentially amorphous state. Such a solid dispersion can thus be referred to as an amorphous solid dispersion. In embodiments, the solid dispersion thus is an amorphous solid dispersion.

In one embodiment, the solid dispersion comprises a predetermined amount of compound (1) or a pharmaceutically acceptable salt thereof. In this context, a predetermined amount refers to the initial amount of compound (1), or a pharmaceutically acceptable salt thereof used for the preparation of the solid dispersion.

In another embodiment, the solid dispersion comprises a therapeutically effective amount of compound (1) or a pharmaceutically acceptable salt thereof.

Solid dispersions of the invention can be prepared starting from form I, III or IV as described herein (in the broadest form or in any embodiment or aspect thereof) or a mixture thereof using any process known in the art for this purpose, for example as disclosed in S. V. Bhujbal et al., Acta Pharmaceutica Sinica B 2021; 11(8):2505e2536, which is herein incorporated by reference. According to the invention, solid dispersions are generally prepared by dissolving an active substance and a pharmaceutically acceptable dispersion carrier in a solvent or mixture of solvents to form a feed solution, and then the solvent is removed from the feed solution, such as by spray-drying, to form the solid dispersion.

A further aspect of the present invention is thus a process of preparing the solid dispersion as described herein, the process comprising the steps of:
a) providing a mixture of compound (1) and a pharmaceutically acceptable dispersion carrier and adding a solvent to obtain a solution or a suspension; and
b) removing the solvent from the solution or the suspension to form the solid dispersion as described herein, wherein

in step a), the compound (1) is provided as any one of crystalline forms I, III, IV as described above in the broadest form or in any embodiment. Preferably, in step b), the solid dispersion comprises compound (1) in amorphous form.

A further aspect of the present invention is thus a process of preparing the solid dispersion as described herein, the process comprising the steps of:
a) providing a mixture of compound (1) and a pharmaceutically acceptable dispersion carrier and adding a solvent to obtain a solution or a suspension; and
b) removing the solvent from the solution or the suspension to form the solid dispersion as described herein, wherein

in step a), the compound (1) is provided as any one of crystalline forms I, III, IV (as described above in the broadest form or in any embodiment) or as a mixture thereof. Preferably, in step b), the solid dispersion comprises compound (1) in amorphous form.

This process may further comprise the step of drying the solid dispersion obtained in step b).

The solid dispersion obtained or obtainable by such process is a further aspect of the present invention.

In an embodiment, a process of preparing the solid dispersion as described herein is provided, the process comprising the steps of:
a) providing a solution or suspension comprising compound (1) or a pharmaceutically acceptable salt thereof, a pharmaceutically acceptable dispersion carrier and at least one solvent; and
b) removing the solvent from the solution or the suspension to form the solid dispersion as described herein; and
c) optionally, drying the solid dispersion obtained in b), wherein in step a), the compound (1) is provided as any one of crystalline forms I, III, IV (as described above in the broadest form or in any embodiment) or as a mixture thereof.

The solution or suspension of step a) according to any of the above-described processes can be referred to as a feed solution.

In embodiments, the removing of the solvent in step b) of the above defined processes is carried out by spray-drying, freeze drying, rotary evaporation, distillation, drum drying and/or vacuum drying. In a preferred embodiment, the removing of the solvent in step b) is carried out by spray-drying. Preferably, in step b), the solid dispersion comprises compound (1) in amorphous form. The term “spray drying” as used herein is used conventionally and broadly and generally refers to any process that involves the atomization of a solution, suspension, slurry, or emulsion containing one or more components of the desired product into droplets by spraying followed by the rapid evaporation of the sprayed droplets into solid powder by hot air at a certain temperature and pressure. Spray drying is a process known to a person skilled in the art. Spray drying is generally performed by dissolving the crystalline form of compound (1) and the pharmaceutically acceptable dispersion polymers in a solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput.

Although the solid dispersion is preferably prepared by conventional spray drying techniques, other techniques known in the art may be used, such as melt extrusion, freeze drying, rotary evaporation, co-precipitation, KinetiSol® Dispersing Technology (KSD), fluidized bed technology, drum drying, vacuum drying or other solvent removal processes.

The processes described above of preparing the solid dispersion as described herein may comprise an additional step between steps a) and b) of spraying the solution or suspension obtained in step a) onto inert excipient cores. This process belongs to fluid bed technology, in particular fluid bed granulation technology.

In an embodiment, a process of preparing the solid dispersion as described herein is provided, the process comprising the steps of:
(a) providing a solution or suspension comprising compound (1) or a pharmaceutically acceptable salt thereof, a pharmaceutically acceptable dispersion carrier and at least one solvent; and
(a′) spraying the solution or suspension provided in (a) onto inert excipient cores; and
(b′) removing the solvent from the inert excipient cores; and
(c′) optionally, drying the excipient cores comprising the solid dispersion obtained in (c′), wherein in step a), the compound (1) is provided as any one of crystalline forms I, III, IV (as described above in the broadest form or in any embodiment) or as a mixture thereof.

The spraying in step (a′) may be performed in a fluidized bed coater e.g. as top spray, bottom spray, Wurster, tangential or side rotor spray.

Any solvent or mixture of solvents where crystalline compound (1) at least partially dissolves can be used. Examples of suitable solvents that can be used individually or as mixtures include water, alcohols, such as methanol (“MeOH”), ethanol (“EtOH”), n-propanol, isopropanol and butanol such as n-butanol, 2-butanol, isobutanol and tert-butanol; ketones, such as acetone, methyl ethyl ketone and methyl isobutyl ketone; esters, such as methyl acetate, ethyl acetate and propyl acetate, isopropyl acetate, n-butyl acetate and isobutyl acetate; and various other solvents, such as dichloromethane (DCM), chloroform, tetrahydrofuran, acetonitrile, toluene and 1,1,1-trichloroethane. In an embodiment, the solvent referred to in any of the above described processes and embodiments thereof is selected from the group consisting of water, alcohols, ketones, esters, dichloromethane, chloroform, tetrahydrofuran, acetonitrile, toluene, 1,1,1-trichloroethane and mixtures thereof. In an embodiment, the solvent referred to in any of the above described processes and embodiments thereof is selected from the group consisting of alcohols (in particular methanol, ethanol, n-propanol, isopropanol and butanol such as n-butanol, 2-butanol, isobutanol and tert-butanol), ketones (in particular acetone, methyl ethyl ketone and methyl isobutyl ketone), esters (in particular methyl acetate, ethyl acetate and propyl acetate, isopropyl acetate, n-butyl acetate and isobutyl acetate), dichloromethane (DCM), tetrahydrofuran, acetonitrile, toluene and 1,1,1-trichloroethane. Mixtures of solvents with water may also be used.

In embodiments, said solvent is a mixture of dichloromethane (DCM) and methanol (MeOH). The relative amounts of DCM and MeOH in the mixture may vary. Preferably, the mixture comprises at least 25 wt % MeOH based on a total weight of 100 wt % of the mixture. In embodiments, the mixture comprises an excess of DCM. Still preferably, the weight:weight ratio of DCM:MeOH ranges from 25:75 to 95:5 (w/w). Preferably, DCM and MeOH are in a weight:weight ratio of approximately 25:75, 50:50, 70:30, 75:25, 80:20, 85:15 or 90:10. A solvent mixture of DCM:MeOH in a ratio of approximately 90:10 (w/w) was advantageously found to enable higher throughput for spray-drying.

In embodiments, the concentration of solids in the feed solution (in particular the suspension or solution as defined in step a) above) is in the range of from about 1 to 20 wt %, based on a total weight of 100 wt % of the feed solution. Preferably, the concentration of solids in the feed solution is in the range of from about 5 to 15 wt %, more preferably of from about 8 to 12 wt % based on a total weight of 100 wt % of the feed solution. For example, the concentration of solids in the feed solution is about 8 wt % or 10 wt %, based on a total weight of 100 wt % of the feed solution.

After removal of the solvent by spray drying, the obtained solid dispersion is optionally subjected to a drying process in order to reduce residual solvent content. In embodiments, drying is performed at a temperature in the range of from about room temperature to 100° C., preferably of from about 30 to 60° C., more preferably of from about 35 to 45° C. For example, the drying is performed at a temperature of about 40° C. In other embodiments, the drying is performed at ambient pressure and/or under reduced pressure. For example, the drying is performed at ambient pressure or at a pressure of about 900 mbar or less, more preferably of about 100 mbar or less and most preferably of about 50 mbar or less, such as about 20 mbar or less. In still other embodiments, the drying is performed for a period in the range of from about 6 to 72 hours, preferably of from about 12 to 48 hours.

Pharmaceutical compositions, such as tablets, preferably film-coated tablets, can be manufactured according to conventional methods known to a skilled person. In embodiments, the manufacturing process can comprise the steps of 1) manufacturing a solid dispersion such as by spray-drying as described herein, 2) dry granulating of the solid dispersion with one or more suitable excipient(s), 3) blending the granules with suitable disintegrant(s) and/or lubricant(s), and/or glidants 4) compressing the blend into tablet cores, and 5) optionally film-coating the tablet cores.

In embodiments of the process of preparing a solid dispersion, compound (1) as crystalline form may be provided in an amount in a range of from 5 wt % to 95 wt %, based on a total weight of 100 wt % of the solid dispersion. In embodiments of the process of preparing a solid dispersion, compound (1) as crystalline form may be provided in an amount in a range of from 25 wt % to 75 wt %, based on a total weight of 100 wt % of the solid dispersion. In embodiments, the pharmaceutically acceptable dispersion carrier may be provided in an amount in a range of from 5 wt % to 95 wt %, based on a total weight of 100 wt % of the solid dispersion. In embodiments, the pharmaceutically acceptable dispersion carrier may be provided in an amount in a range of from 25 wt % to 75 wt %, based on a total weight of 100 wt % of the solid dispersion.

In embodiments of the process of preparing a solid dispersion, compound (1) as crystalline form may be provided in an amount in a range of from 20 wt % to 50 wt %, based on a total weight of 100 wt % of the solid dispersion. In embodiments, compound (1) as crystalline form may be provided in an amount in a range of from 25 wt % to 50 wt %, based on a total weight of 100 wt % of the solid dispersion. In embodiments, the pharmaceutically acceptable dispersion carrier may be provided in an amount in a range of from 50 wt % to 80 wt %, based on a total weight of 100 wt % of the solid dispersion. In embodiments, the pharmaceutically acceptable dispersion carrier may be provided in an amount in a range of from 50 wt % to 80 wt %, based on a total weight of 100 wt % of the solid dispersion. In embodiments, the pharmaceutically acceptable dispersion carrier may be provided in an amount in a range of from 50 wt % to 75 wt %, based on a total weight of 100 wt % of the solid dispersion.

In embodiments, the compound (1) and the pharmaceutically acceptable dispersion carrier may be provided in approximately equal weight amounts. In embodiments, approximately 50 wt % of compound (1) and approximately 50 wt % of the pharmaceutically acceptable dispersion carrier are provided.

In embodiments, approximately 25 wt % or 50 wt % of compound (1) and approximately 75 wt % or 50 wt % of the pharmaceutically acceptable dispersion carrier are provided.

In embodiments, the weight ratio of compound (1): the pharmaceutically acceptable dispersion carrier in the process of preparing a solid dispersion, in particular in in the solution or suspension of step a), is of approximately 1:4 to 4:1, preferably 1:3 to 3:1, such as 1:1 to 1:3. In embodiments, the weight ratio of compound (1) and the pharmaceutically acceptable dispersion carrier in the process of preparing a solid dispersion, in particular in the solution or suspension of step a), is of approximately 1:1.

Use for Treatment and/or Prevention of Oncological and/or Hyperproliferative Diseases

The crystalline forms I, III, IV as described above in the broadest form or in any embodiment can be used as a medicament. Particularly, the crystalline forms I, III, IV as described above in the broadest form or in any embodiment can be used for the treatment and/or prevention of oncological and/or hyperproliferative disorders, in particular in anti-cancer therapy.

According to an aspect is provided crystalline form I, as described above in the broadest form or in any embodiment, for use as a medicament. According to an aspect is provided crystalline form III, as described above in the broadest form or in any embodiment, for use as a medicament. According to an aspect is provided crystalline form IV, as described above in the broadest form or in any embodiment, for use as a medicament. According to an aspect is provided a mixture of crystalline form I, III and/or IV, as described above in the broadest form or in any embodiment, for use as a medicament.

According to an aspect is provided crystalline form I, as described above in the broadest form or in any embodiment, for use as an anti-cancer medicament. According to an aspect is crystalline form III, as described above in the broadest form or in any embodiment, for use as an anti-cancer medicament. According to an aspect is crystalline form IV, as described above in the broadest form or in any embodiment, for use as an anti-cancer medicament.

In an embodiment is provided crystalline form I, III or IV as described above (in the broadest form or in any embodiment) or a mixture thereof for use in the treatment and/or prevention of a disease or a disorder modulated by HER2, particularly an oncological and/or hyperproliferative disease. Another aspect refers to crystalline form I, III or IV as described above (in the broadest form or in any embodiment) or a mixture thereof for use in a method of treating and/or preventing a disease or disorder modulated by HER2, particularly an oncological or hyperproliferative disease.

A further aspect relates to a method of treating and/or preventing a disease or disorder modulated by HER2, particularly an oncological and/or hyperproliferative disease, wherein the method comprises the step of administering crystalline form I, III or IV as described above (in the broadest form or in any embodiment) or a mixture thereof to a patient. In an embodiment, such method comprises administering to a human in need of such treatment a therapeutically effective amount of crystalline form I, III or IV as described above (in the broadest form or in any embodiment) or a mixture thereof.

A related aspect relates to the use of crystalline form I, III or IV as described above (in the broadest form or in any embodiment) or a mixture thereof in the manufacture of a medicament. An embodiment relates to the use of crystalline form I, III or IV as described above (in the broadest form or in any embodiment) or a mixture thereof in the manufacture of a medicament for the treatment and/or prevention of a disease or disorder modulated by HER2, particularly an oncological and/or hyperproliferative disease.

In one aspect, is provided crystalline form I, III or IV as described above (in the broadest form or in any embodiment) or a mixture thereof for use in the treatment and/or prevention of a disease and/or condition, wherein the inhibition of wild type and/or mutant HER2 is of therapeutic benefit, particularly for the treatment and/or prevention of a disease and/or condition, wherein the inhibition of HER2 exon 20 mutant protein is of therapeutic benefit. Examples of such diseases and/or conditions include, but are not limited to, oncological and/or hyperproliferative diseases such as cancer.

One aspect relates to crystalline form I, III or IV as described above (in the broadest form or in any embodiment) or a mixture thereof for use in the treatment and/or prevention of an oncological and/or hyperproliferative disease.

As used herein, the term “hyperproliferative disease” refers to conditions wherein cell growth is increased over normal levels. Hyperproliferative diseases include malignant diseases, such as cancers, and non-malignant diseases. In preferred embodiments, the hyperproliferative disorder is cancer. As used herein, the term “oncological disease” refers to a disease or medical condition associated with cancer or cancer indication. Cancers can be classified by the type of tissue in which the cancer originates (histological type) and by primary site, or the location in the body, where the cancer first developed.

In an embodiment, the oncological and/or hyperproliferative disease is cancer.

In an embodiment is provided crystalline form I, as described above in the broadest form or in any embodiment, for use in the treatment and/or prevention of cancer. In an embodiment is provided crystalline form III, as described above in the broadest form or in any embodiment, for use in the treatment and/or prevention of cancer. In an embodiment is provided crystalline form IV, as described above in the broadest form or in any embodiment, for use in the treatment and/or prevention of cancer. In an embodiment is provided a mixture of crystalline form I, III and/or IV, as described above in the broadest form or in any embodiment, for use in the treatment and/or prevention of cancer.

Another aspect refers to crystalline form I, III or IV as described above (in the broadest form or in any embodiment) or a mixture thereof for use in a method of treating and/or preventing cancer.

A further aspect relates to a method of treating and/or preventing cancer, wherein the method comprises the step of administering crystalline form I, III or IV as described above (in the broadest form or in any embodiment) or a mixture thereof to a patient. In an embodiment, such method comprises administering to a human in need of such treatment a therapeutically effective amount of crystalline form I, III or IV as described above (in the broadest form or in any embodiment) or a mixture thereof.

An embodiment relates to the use of crystalline form I, III or IV as described above (in the broadest form or in any embodiment) or a mixture thereof in the manufacture of a medicament for the treatment and/or prevention of cancer.

In embodiments, the cancer is HER2 overexpressed, HER2 amplified and/or HER2 mutant. In embodiments, the cancer is HER2 exon 20 mutant cancer.

In embodiments, the oncological and/or hyperproliferative disease is a HER2 overexpressed, HER2 amplified and/or HER2 mutant cancer.

“HER2 overexpressed” as used herein refers to a cancer, where the cells of the cancer or tumor express HER2 at levels detectable by immunohistochemistry (e.g. IHC 2+ or IHC 3+) and/or methods assaying ERBB2 messenger RNA.

“HER2 amplified” as used herein refers to a cancer where the cancer or tumor cells exhibit more than 2, in particular more than 3, 4, 5, 6, 7, 8, 9 or 10, preferably more than 6, copies of the HER2 gene ERBB2.

HER2 expression, gene copy number and amplification can be measured, for example, by determining nucleic acid sequencing (e.g., sequencing of genomic DNA or cDNA), measuring mRNA expression, measuring protein abundance, or a combination thereof. HER2 testing methods include immunohistochemistry (IHC), fluorescence in situ hybridization (FISH), chromogenic in situ hybridization (CISH), ELISAs, and RNA quantification using techniques such as RT-PCR, microarray analysis and Next Generation Sequencing (NGS). HER2 expression in or on the cancer sample cells can be compared to a reference cell. The reference cell can be a non-cancer cell obtained from the same subject as the sample cell. The reference cell can be a non-cancer cell obtained from a different subject or a population of subjects. When the cancer is HER2 overexpressed and/or HER2 amplified in or on a cell, the cancer can be referred to as being “HER2 positive”.

“HER2 mutant” as used herein refers to a cancer harbouring at least one mutation, i.e. an alteration in the nucleic acid sequence of the HER2 gene and/or an alteration in the amino acid sequence of the HER2 protein, including but not limited to those listed below. Mutations can be found with any method known to the skilled person, such as molecular diagnostic methods including but not limited to Polymerase Chain Reaction (PCR), Single Strand Conformational Polymorphism (SSCP), Denaturing Gradient Gel Electrophoresis (DGGE), Heteroduplex analysis, Restriction fragment length polymorphism (RFLP), Next Generation Sequencing (NGS) and Whole Exome Sequencing.

“Cancer with HER2 exon 20 mutation” or “HER2 exon 20 mutant cancer” as used herein refers to a cancer where the cancer or tumor cells harbour at least one HER2 exon 20 mutation including but not limited to the mutations listed below.

ERBB2 (HER2) exon 20 encodes for a part of the kinase domain and ranges from amino acids 769 to 835. Every mutation, insertion, duplication or deletion within this region is defined as an exon 20 mutation including the following mutations: p.A772_G773insMMAY; p.Y772_A775_dup (YVMA); p.A775_G776ins YVMA; p. Y772ins YVMA; p.M774delinsWLV; p.A775_G776insSVMA; p.A775_G776insVVMA; p.A775_G776insYVMS; p.A775_G776insC; p.A776_delinsVC; p.A776_delinsLC; p.A776_delinsVV; p.A776_delinsAVGC; p.A776_delinsIC; p.A776_V777delinsCVC; p.V777_insE; p.G778_P780dup (GSP); p.G776_delinsVC (“p.” is referring to the HER2 protein).

In addition oncogenic HER2 mutations exist outside of exon 20 including the following mutations: p.S310F; p.R678Q; p.L755S; p.L755A; p.L755P; p.S310Y; p.S310A; p.V842I; p.D769Y; p.D769H; p.R103Q; p.G1056S; p.1767M; p.L869R; p.L869R; p.T733I; p.T862A; p. V697L; p.V777L; p. V777M; p.R929W; p.D277H; p.D277Y; p.G660D (“p.” is referring to the HER2 protein).

In embodiments, the oncological and/or hyperproliferative disease or the cancer is one of the following cancers, tumors or other proliferative diseases, without being restricted thereto: Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis); appendix;
cancers/tumors/carcinomas of the testis: e.g. seminomas, non-seminomas;
Gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; nonfluorineunctional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
peripheral nervous system cancer;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (BchlorineL), chronic T-cell lymphocytic leukemia (TchlorineL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP).

All cancers/tumors/carcinomas mentioned above which are characterized by their specific location/origin in the body are meant to include both the primary tumors and the metastatic tumors derived therefrom. Preferably, the cancer as defined herein (including in any embodiment referring to e.g. cancer types) is metastatic, advanced, and/or unresectable.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

In some embodiments, the cancer is selected from the group consisting of brain cancer, breast cancer, endocrine cancer, gastrointestinal cancer, gynecologic cancer, head and neck tumor, lung cancer, nervous system cancer, and skin cancer.

Preferably, said brain cancer is a glioblastoma or a glioma.

Preferably, said breast cancer is lobular breast cancer. In addition or in alternative, said breast cancer is preferably metastatic.

Preferably, said endocrine cancer is nerve sheath tumor, more preferably HER2 mutant nerve sheath tumor.

Preferably, said gastrointestinal cancer is selected from the group consisting of anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer and small bowel cancer. In addition or in alternative, said gastrointestinal cancer may be a gastrointestinal neuroendocrine tumor, preferably HER2 mutant. Still preferably, said gastrointestinal cancer is selected from the group consisting of gastric adenocarcinoma, gastroesophageal junction adenocarcinoma and esophageal adenocarcinoma, in particular metastatic gastric adenocarcinoma, metastatic gastroesophageal junction adenocarcinoma and metastatic esophageal adenocarcinoma.

Preferably, said gynecologic cancer is selected from the group consisting of cervical cancer, uterine cancer, endometrial cancer and ovarian cancer.

Preferably, said head and neck tumor is a salivary gland cancer or tumor.

Preferably, said lung cancer is non-small cell lung cancer (NSCLC).

Preferably, said nervous system cancer is peripheral nervous system cancer, more preferably HER2 amplified peripheral nervous system cancer.

Preferably, said skin cancer is not a melanoma, i.e. non-melanoma skin cancer.

In some embodiments, the cancer is selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer. In some embodiments, the cancer is HER2 overexpressed, HER2 amplified and/or HER2 mutant (in particular HER2 exon 20 mutant) cancer selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In some embodiments, the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In some embodiments, the cancer is HER2 overexpressed, HER2 amplified and/or HER2 mutant (in particular HER2 exon 20 mutant) cancer selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In some embodiments, the cancer is selected from the group consisting of brain cancer, breast cancer, biliary cancer, bladder cancer, cervical cancer, uterine cancer, colorectal cancer, endometrial cancer, skin cancer, gastric cancer, esophagus tumor, head and neck tumor, gastrointestinal cancer, gallbladder tumor, kidney cancer, liver cancer, lung cancer and prostate cancer.

In embodiments, the cancer is HER2 overexpressed, HER2 amplified and/or HER2 mutant (in particular HER2 exon 20 mutant) cancer selected from brain cancer, breast cancer, biliary cancer, bladder cancer, cervical cancer, uterine cancer, colorectal cancer, endometrial cancer, skin cancer, gastric cancer, esophagus tumor, head and neck tumor, gastrointestinal cancer, gallbladder tumor, kidney cancer, liver cancer, lung cancer and prostate cancer.

In other embodiments, the cancer is selected from the group consisting of breast cancer, bladder cancer, colorectal cancer, gastrointestinal cancer, esophageal cancer or lung cancer. In further embodiments, the cancer is selected from cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer). In still further embodiments, the cancer is NSCLC. In still further embodiments, the cancer is HER2 exon 20 mutant NSCLC.

In an embodiment, the cancer is advanced, unresectable or metastatic NSCLC harbouring a HER2 mutation, wherein said HER2 mutation is in the tyrosine kinase domain. Preferably, in this embodiment, the solid dispersion or the pharmaceutical composition as described herein is administered as first line of therapy. Still preferably, in this embodiment, the solid dispersion or the pharmaceutical composition as described herein is administered as second or further line of therapy.

In embodiments, the cancer is HER2 positive metastatic breast cancer. Preferably, in this embodiment, the solid dispersion or the pharmaceutical composition as described herein is administered as first line of therapy. Still preferably, in this embodiment, the solid dispersion or the pharmaceutical composition as described herein is administered as second or further line of therapy.

In embodiments, the cancer is HER2 positive metastatic gastric adenocarcinoma, metastatic gastroesophageal junction adenocarcinoma or metastatic esophageal adenocarcinoma. Preferably, in this embodiment, the solid dispersion or the pharmaceutical composition as described herein is administered as first line of therapy. Still preferably, in this embodiment, the solid dispersion or the pharmaceutical composition as described herein is administered as second or further line of therapy.

In another aspect, the present invention relates to crystalline form I, III or IV as described above (in the broadest form or in any embodiment) or a mixture thereof for use in the treatment and/or prevention of an oncological and/or hyperproliferative disease as defined herein, wherein the crystalline form I, III or IV or mixture thereof is administered in combination with a cytostatic and/or cytotoxic active substance and/or in combination with radiotherapy and/or immunotherapy.

In another aspect, the present invention relates to a combination of crystalline form I, III or IV as described above (in the broadest form or in any embodiment) or a mixture thereof with a cytostatic and/or cytotoxic active substance and/or in combination with radiotherapy and/or immunotherapy for use in the treatment and/or prevention of cancer.

Crystalline form I, III or IV as described above (in the broadest form or in any embodiment) may be used on their own or in combination with one or more other pharmacologically active substances such as state-of-the-art or standard-of-care compounds, such as e.g. cell proliferation inhibitors, anti-angiogenic substances, steroids or immune modulators/checkpoint inhibitors, and the like.

Pharmacologically active substances which may be administered in combination with the crystalline forms as described herein, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib, bevacizumab, pertuzumab and trastuzumab); antimetabolites (e.g. antifolates such as methotrexate, raltitrexed, pyrimidine analogues such as 5fluorouracil (5fluorineU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, KRAS inhibitors (e.g. KRAS G12C inhibitors), signalling pathway inhibitors (e.g. SOS1 inhibitors), FLT3 inhibitors, BRD4 inhibitors, IGF-1R inhibitors, TRAILR2 agonists, Bcl-xL inhibitors, Bcl-2 inhibitors, Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), T-cell engagers (e.g. bi-specific T-cell engagers (BiTEs®) like e.g. CD3×BCMA, CD3×CD33, CD3×CD19), PSMA×CD3), tumor vaccines and various chemotherapeutic agents such as amifostin, anagrelid, clodronat, filgrastin, interferon, interferon alpha, leucovorin, procarbazine, levamisole, mesna, mitotane, pamidronate and porfimer.

### Pharmaceutical Composition

According to a further aspect, the present invention relates to a pharmaceutical composition comprising crystalline form I, III or IV as described above (in the broadest form or in any embodiment) and one or more pharmaceutically acceptable excipients.

According to a further aspect, the present invention relates to a pharmaceutical composition comprising a therapeutically effective amount of crystalline form I, III or IV as described above (in the broadest form or in any embodiment) or a mixture thereof and one or more pharmaceutically acceptable excipients. Another embodiment of the present invention is a pharmaceutical composition comprising a therapeutically effective amount of the solid dispersion as described herein and one or more pharmaceutically acceptable excipients.

The term “therapeutically effective amount” as used herein refers to a quantity of substance that is capable of obviating symptoms of illness or of preventing or alleviating these symptoms, or which prolong the survival of a treated patient.

The term “pharmaceutically acceptable excipient” refers to a non-toxic component that does not destroy the pharmacological activity of the compound with which it is formulated. Pharmaceutically acceptable excipients that may be used in the compositions of this invention include fillers, disintegrants, glidants, lubricants, and coating agents. The compositions may comprise further pharmaceutically acceptable excipients selected from buffers, dispersion agents, surfactants, wetting agents, emulsifiers, suspending agents, preservatives, antioxidants, opaquing agents, processing aids, colorants, sweeteners, perfuming agents, flavoring agents, diluents and other known additives usable in the manufacturing of a pharmaceutical product. The pharmaceutical composition may contain conventional non-toxic pharmaceutically acceptable excipients. In embodiments of the pharmaceutical composition, the one or more pharmaceutically acceptable excipients are selected from the group of fillers, disintegrants, glidants, lubricants, and coating agents. In embodiments, the pharmaceutical composition comprises a filler, a disintegrant, a glidant and a lubricant. In embodiments, the pharmaceutical composition comprises a filler, a disintegrant, a glidant, a lubricant and a coating agent. It is to be understood that the pharmaceutical composition may comprise one or more excipients of each function, e.g. one or more filler, one or more disintegrants, one or more glidants, one or more lubricants, one or more coating agents.

In embodiments, the filler(s) is(are) selected from the group consisting of microcrystalline cellulose, mannitol and mixtures thereof. In embodiments, the disintegrant(s) is(are) selected from the group consisting of crosslinked sodium carboxymethyl cellulose, also denoted croscarmellose, or sodium bicarbonate, crospovidone, sodium starch glycolate and mixtures thereof. In certain embodiments, the disintegrant is croscarmellose sodium. In embodiments, the glidant is colloidal silicon dioxide. In embodiments, the lubricant(s) is(are) selected from the group consisting of stearyl fumarate, magnesium stearate and mixtures thereof. In certain embodiments, the lubricant is sodium stearyl fumarate.

In embodiments of the pharmaceutical composition the one or more pharmaceutically acceptable excipients comprise mannitol, microcrystalline cellulose, croscarmellose sodium, colloidal silicon dioxide and sodium stearyl fumarate.

In certain embodiments, the pharmaceutical composition may comprise a coating agent, such as when formulated as a film-coated tablet. In embodiments, the coating agent may comprise film-forming agents such as polyvinyl alcohol that may be partially hydrolysed, anti-tacking agents such as talc, pigments such as titanium dioxide, glyceryl mono and dicaprylocaprate (GMDCC) and iron oxides such as iron oxide yellow, and lubricants such as sodium lauryl sulphate. Coating agents are commercially available such as under the tradename Opadry™ AMB II yellow. In a preferred embodiment, the coating agent does not contain titanium dioxide e.g. is free of titanium dioxide.

The crystalline form present in the solid dispersion may be as defined in any of the above aspects, objects and/or embodiments and thus especially according to at least one of crystalline forms I, III, and IV.

The crystalline form present in the pharmaceutical composition may be as defined in any of the above aspects, objects and/or embodiments and thus especially according to at least one of crystalline forms I, III, and IV.

In particular, the invention provides a pharmaceutical composition comprising crystalline form I as defined herein and one or more pharmaceutically acceptable excipients.

In particular, the invention provides a pharmaceutical composition comprising crystalline form III as defined herein and one or more pharmaceutically acceptable excipients.

In particular, the invention provides a pharmaceutical composition comprising crystalline form IV as defined herein and one or more pharmaceutically acceptable excipients.

In an embodiment, the pharmaceutical composition comprising the crystalline form as defined herein, comprises one or more pharmaceutically acceptable excipients and an additional therapeutic agent.

According to a further embodiment, the pharmaceutical composition comprises at least one other cytostatic and/or cytotoxic active substance.

Suitable preparations for administering the compounds of the invention will be apparent to those with ordinary skill in the art and include for example tablets, pills, capsules, suppositories, lozenges, troches, solutions—particularly solutions for injection (s.c., i.v., i.m.) and infusion (injectables)—elixirs, syrups, sachets, emulsions, inhalatives or dispersible powders. Suitable tablets may be obtained, for example, by mixing one or more compounds of the crystallic forms with known excipients, for example inert diluents, carriers, disintegrants, adjuvants, surfactants, binders and/or lubricants.

The dosage range of the crystalline forms as described applicable per day is usually from 1 mg to 2000 mg, preferably from 10 to 1000 mg.

The dosage for intravenous use is from 1 mg to 1000 mg with different infusion rates, preferably between 5 mg and 500 mg with different infusion rates.

However, it may sometimes be necessary to depart from the amounts specified, depending on the body weight, age, the route of administration, severity of the disease, the individual response to the drug, the nature of its formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering large amounts it may be advisable to divide them up into a number of smaller doses spread over the day Unless otherwise defined, the technical and scientific terms used herein have the same meaning as commonly understood by one of ordinary skill in the art to which this invention belongs. It is to be understood that any of the above illustrated aspect or embodiment can be combined with any other of the above illustrated aspect or embodiment to provide an additional aspect or embodiment. In particular, for form IV, each embodiment characterizing the crystalline form by its X-ray diffraction pattern can be combined with any other embodiment characterizing the crystalline form by its Raman spectrum to provide a further embodiment, where the crystalline form is characterized by its X-ray diffraction pattern and its Raman spectrum. The same applies to forms III and I.

In many embodiments described above, peaks comprised in an X-ray diffraction pattern (i.e. X-ray diffraction peaks) are cited as an absolute value followed by an error range, for instance (6.2±0.2°), where 6.2 is the absolute value and +0.2 is the error range, such that the peak lies between 6.0° and 6.4°. The error range is usually ±0.1 or +0.2. For all aspects or embodiments with an error range of +0.2 referred to XRPD, corresponding embodiments with an error range of +0.1 are herein disclosed. This means that in any aspect or embodiment referring to an error range of +0.2, 0.2 can be replaced by 0.1 to provide additional embodiments of the present invention.

Similarly, in many embodiments described above, peaks comprised in Raman spectrum (i.e. Raman peaks) are cited as an absolute value followed by an error range, for instance 831±2, where 831 is the absolute value and +2 is the error range, such that the peak lies between 829 and 833. The error range is usually ±2. For all aspects or embodiments with an error range of +2 referred to Raman, corresponding embodiments with an error range of +1 are herein disclosed. This means that in any aspect or embodiment referring to an error range of +2, 2 can be replaced by 1 to provide additional embodiments of the present invention.

In all embodiments referring to X-ray diffraction peaks, the peaks preferably have more than 5% relative intensity, especially wherein the X-ray diffraction pattern is obtained with the experimental parameters reported in Table 1.

Features and advantages of the present invention will become apparent from the following detailed examples, which illustrate the principles of the invention by way of example without restricting its scope:

### EXAMPLES

### Abbreviations

The following abbreviations are used herein.
° C. degrees Celsius
2-Me-THF 2-methyltetrahydrofuran
AcCl acetyl chloride
Boc tert-butoxycarbonyl
cat. catalytic
DCM dichloromethane
DMSO dimethyl sulfoxide
DMSO-d6 deuterated dimethyl sulfoxide
DMAc N,N-dimethylacetamide
equiv. equivalent or equivalents
Et₃N triethylamine
h, hr or hrs hour or hours
HCl hydrochloric acid
IPA iso-propanol
IPAc iso-propyl acetate
KOH potassium hydroxide
LR EI MS low resolution electron ionization mass spectroscopy
m, min or mins minute or minutes
M molar
Me methyl
MeCN acetonitrile
MeOH methanol
mol % mole or moles percent
MS mass spectroscopy
NaOH sodium hydroxide
NMR nuclear magnetic resonance
PG protecting group
ROI residue on ignition
THF tetrahydrofuran
V volume or volumes

Solid crystalline forms of compound (1) can be produced as polymorphic forms I, III, and IV. Examples for respective methods for producing the defined polymorphic forms are given below in Examples 1 and 2.

### Example 1—Preparation of Crystalline Forms of Compound (1)

### General

Unless stated otherwise, all reactions are carried out in commercially available apparatus using methods that are commonly used in chemical laboratories. Starting materials that are sensitive to air and/or moisture are stored under protective gas and corresponding reactions and manipulations therewith are carried out under protective gas (nitrogen or argon). If a compound is to be represented both by a structural formula and by its nomenclature, in the event of a conflict the structural formula is decisive.

### Analytical Methods

¹H NMR spectra are recorded in dimethyl sulfoxide-d6 (DMSO-d6) on Bruker (400 MHZ) spectrometer.

MS measurements are carried out using Waters ACQuITY QDa Detector paired with LC System. Ionization Parameters are as follows: 100-800 mass range; 15V cone voltage; 15 pts/s sampling rate; 0.8V +/−capillary voltage; 600° C. probe temperature.

HPLC Method for measuring compound (5a) and compound (5b) ratio:

HPLC equipped with gradient pump (600 bar), column thermostat, UV-detector and autosampler thermostat.
Column: Waters XBridge BEH C18 4.6×100 mm, 2.5 μm (or an equivalent column)
Solvents and Reagent Acetonitrile, HPLC grade
   Water, Milli-Q water or equivalent
   Ammonium formate, ≥99%
   Formic acid, LC-MS grade
   Methanol, HPLC grade
Buffer Preparation Accurately weigh 0.63 g of ammonium formate into a 1L volumetric flask.
   Dissolve in 800 mL of water. Adjust pH to 2.80 using formic acid and dilute to volume with water.
   Filter the ammonium formate buffer before use.
   Assemble the filter device with a C18 filter disk (Supelco Empore or equivalent, 47 mm diameter). Install the filtration device to a waste flask.
   Make sure that the surface of the filter is covered with liquid (1-2 mm thickness) always during the activation and filtration procedure. If the filter runs dry, re-start the procedure from activation Step 1.
   Activation step 1: Pump 30-50 mL MeOH through the filter.
   Activation step 2: Pump 30-50 mL of MilliQ water through the filter.
   Filtration: Install the filtration device to a clean flask and filtrate the mobile phase.
Mobile Phase A Buffer
Mobile Phase B Acetonitrile
Diluent Acetonitrile
Flow 1.2 mL/min
Gradient

| Time, [min] | Mobile Phase B % |
|---|---|
| 0 | 10 |
| 5 | 30 |
| 15 | 50 |
| 18 | 90 |
| 18.1 | 10 |
| 20 | 10 |

Wavelength 254 nm
Column temperature 40° C.
Sample Temperature 5° C.
Retention Times:

| Component | Retention Times, [min] |
|---|---|
| Compound (5a) | 1.00 |
| Compound (5b) | 1.10 |

### Example 1.1—Route 1 to Obtain Crystalline Forms I and III

Scheme 1. Synthesis of Compound (1) from Compound (9), Compound (10), and Compound (6b), Based on a Halogen Leaving Group

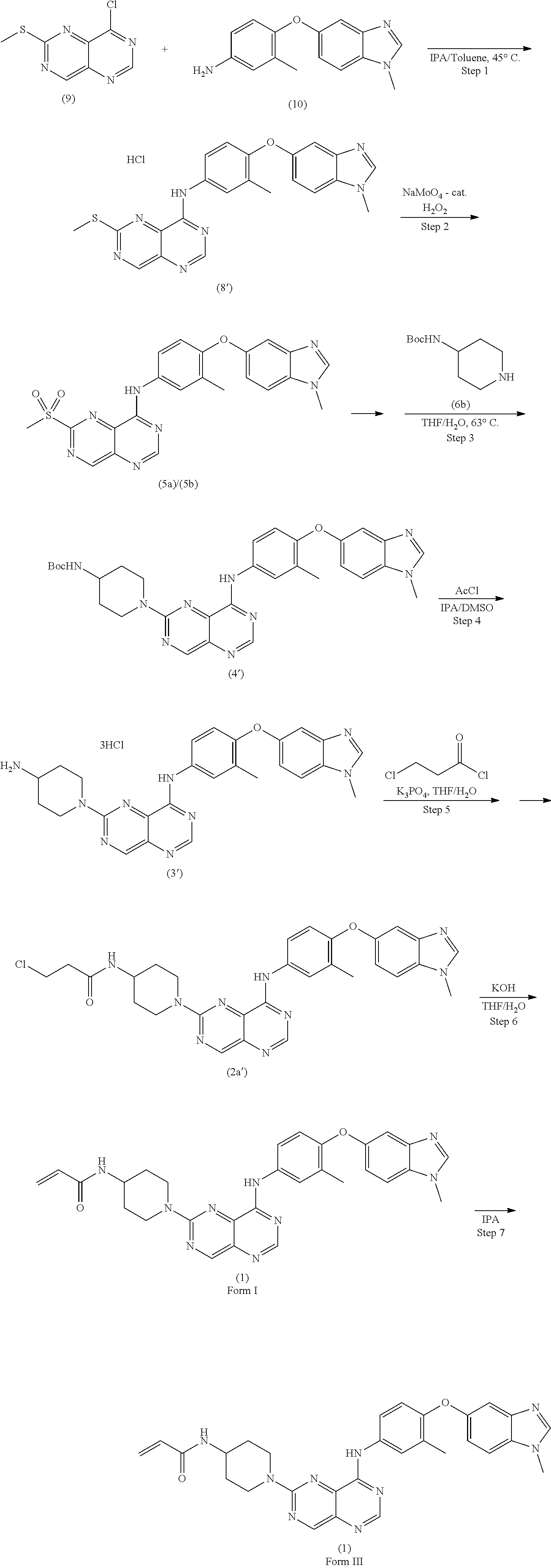

Starting materials (9) and (10) are commercially available and can be prepared according to procedures known in the art. For example, compound (9) can be prepared as described in WO 97/32880, WO 2010/026262 or WO 2020/239999 and compound (10) can be prepared as described in WO 2019/214634, WO 2021/156178, WO 2021/213800 or WO 2022/003575.

Step 1. Preparation of Compound (8′) from Compound (9) and Compound (10).

### General Procedure.

All calculations are made with respect to compound (9).

To a clean N₂-sparged vessel, compound (9) (1.0 equiv.), compound (10) (1.0 equiv.), and toluene (2.0 V) are charged. Agitation is started and IPA (10.0 V) is added. Upon completion of addition, mixture is heated to 43° C. and held until reaction is complete. Then, the reaction mixture is cooled to 22° C., agitated for 30 minutes and filtered. Solids are washed with IPA (1.5 V) twice and dried in vacuum at 50° C. to yield compound (8′) as a solid in 93% yield.

### Analytical Information

compound (8′): ¹H NMR (400 MHZ, DMSO-d₆) δ ppm 2.21 (s, 3H) 2.77 (s, 3H) 4.05 (s, 3H) 7.09 (d, J=8.76 Hz, 1H) 7.15 (d, J=2.50 Hz, 1H) 7.34 (dd, J=9.01, 2.25 Hz, 1H) 7.85 (dd, J=8.63, 2.63 Hz, 1H) 7.89 (d, J=2.50 Hz, 1H) 7.97 (d, J=9.01 Hz, 1H) 8.66 (s, 1H) 9.27 (s, 1H) 9.48 (s, 1H) 10.04 (br s, 1H); LR EI MS m/z: 430.14

Step 2. Preparation of Compound (5a)/(5b) from Compound (8′).

### General Procedure.

All calculations are made with respect to compound (8′).

To a clean N₂-sparged vessel, compound (8′) (1.0 equiv., HCl salt) and ethanol (5.5 V) are charged. Agitation is started, content is sparged with N₂ and internal temperature of vessel is adjusted to 25° C. To this mixture, a solution of Na₂MoO₄ (1.1 mol %) in water (2.2 V) is charged followed by 30% aq. H₂O₂ (1.20 equiv.) while maintaining an internal temperature of vessel of 25° C. Upon completion of addition, mixture is agitated at 25° C. no less than 3 hours. Then, excess of H₂O₂ is quenched with Na-L-Ascorbate (0.10 equiv.) solution in water (0.3 V). Mixture is agitated for 15 minutes at 25° C. followed by addition of DMSO (6.1 V) and water (0.2 V). pH of the mixture is adjusted to 5.0-6.0 using triethylamine and the mixture is heated to 40° C. followed by addition of water (6 V) while maintaining internal temperature of vessel of 40° C. Then, mixture is brought to 25° C. and solids are filtered. Solids are washed with a solution of water (2.0 V) and ethanol (0.5 V) and dried in vacuum at ambient temperature to yield compound (5a)/(5b) as a solid in 94% yield and 95:5 to 70:30 respected ratio as measured by the analytical method reported above.

### Analytical Information

compound (5a): ¹H NMR (400 MHZ, DMSO-d₆) δ ppm 2.27 (s, 3H) 3.10 (s, 3H) 3.84 (s, 4H) 6.88 (d, J=8.76 Hz, 1H) 7.02 (dd, J=8.69, 2.19 Hz, 1H) 7.13 (d, J=2.25 Hz, 1H) 7.59 (d, J=8.75 Hz, 1H) 7.73 (dd, J=8.69, 2.56 Hz, 1H) 0.00 (d, J=6.50 Hz, 1H) 8.21 (s, 1H) 8.78 (s, 1H) 9.60 (s, 1H) 10.46 (s, 1H); LR EI MS m/z: 446.15

Step 3. Preparation of Compound (4′) from Compound (5a)/(5b) and Compound (6b).

### General Procedure.

Stoichiometry calculations of compound (6b) are based on the calculated content of compound (5a) and compound (5b). The calculated content is obtained by subtracting residual solvents, KF, ROI, and total impurities from the theoretical value of 100%, rather than comparing it to a reference standard with known potency. Calculations of THE amount are based on the weight input of the mixture of compound (5a) and compound (5b).

To a clean N2-sparged Vessel, compound (5a)/(5b) (1.0 equiv.), compound (6b) (1.3 equiv.) and THF (9.0 V) are charged, and agitation is started. Mixture is heated to 60° C. and agitated for no less than 6 hours. Upon completion of reaction, mixture is cooled to −10° C., agitated for an hour and filtered. Wet cake is re-charged to the vessel and triturated with THF (2 V) at 5° C. for an hour, then filtered and washed with THF (1 V). Product is dried in vacuum at ambient temperature. Yield: 87%.

### Analytical Information

compound (4′): ¹H NMR (400 MHZ, DMSO-d₆) δ ppm 1.40 (m, 11H) 1.86 (br d, J=10.01 Hz, 2H) 2.26 (s, 3H) 3.17 (br t, J=11.38 Hz, 2H)-3.50-3.70 (m, 2H) 3.84 (s, 3H)-4.69-5.06 (br. S., 2H)-6.86-6.95 (m, 2H) 7.00 (dd, J=8.63, 2.38 Hz, 1H) 7.10 (d, J=2.25 Hz, 1H) 7.57 (d, J=8.76 Hz, 1H) 7.78 (dd, J=8.76, 2.50 Hz, 1H) 7.84 (d, J=2.25 Hz, 1H) 8.18 (s, 1H) 8.38 (s, 1H) 9.06 (s, 1H) 9.57 (s, 1H); LR EI MS m/z: 582.25

Step 4. Preparation of Compound (3′) from Compound (4′).

### General Procedure.

All calculations are made with respect to compound (4′).

To a clean N2-sparged Vessel #1, IPA (6.0 V) is charged, and agitation is started. Then, acetyl chloride (7.50 equiv.) is charged while maintaining internal temperature of vessel below 45° C. Mixture is heated to 65° C. and agitated for about 1 hour.

To a clean N2-sparged Vessel #2, compound (4′) (1 equiv.) and DMSO (4.0 V) are charged, and mixture is agitated to obtain uniform slurry. The content of the Vessel #2 is charged to Vessel #1 while maintaining internal temperature of vessel at 65° C. Reaction is agitated for no less than 5 hours, then, cooled to ambient temperature and filtered. Solids are washed with IPA (2.0 V) and dried in vacuum at 50° C. to yield compound (3′) in quantitative yield.

### Analytical Information

compound (3′): ¹H NMR (400 MHZ, DMSO-d₆) δ ppm-1.51-1.72 (m, 2H) 2.10 (br d, J=9.76 Hz, 2H) 2.24 (s, 3H) 3.17 (m, 2H)-3.29-3.51 (m, 1H) 4.07 (s, 3H)-4.82-5.32 (broad signal) 7.13 (d, J=8.50 Hz, 1H) 7.17 (d, J-2.25 Hz, 1H) 7.39 (dd, J=9.01, 2.25 Hz, 1H)-7.76-7.86 (m, 2H) 8.01 (d, J=9.01 Hz, 1H) 8.41 (br d, J-3.50 Hz, 3H) 8.62 (s, 1H) 9.21 (s, 1H) 9.57 (s, 1H) 10.53 (br s, 1H); LR EI MS m/z: 482.29

Step 5. Preparation of Compound (2a′) from Compound (3′).

### General Procedure.

All calculations are made with respect to compound (3′).

To a clean N₂-sparged Vessel, compound (3′) (1.0 equiv.) and THF (8.0 V) are charged and agitation is started. To the mixture, a solution of K₃PO₄-5H₂O (4.0 equiv.) in water (6.0 V) is charged while maintaining internal temperature of vessel of 22° C. Mixture is agitated until full dissolution (e.g. between 15 minutes and 1 hr depending on the scale), then, agitation is stopped, layers are settled and separated. To organic layer, a solution of K₃PO₄-5H₂O (1.02 equiv.) in water (1.5 V) is charged, agitation started, and mixture is cooled to 6° C. To this mixture a solution of 3-chloropropionyl chloride (1.02 equiv.) in anhydrous THF (0.92 V) is charged while maintaining internal temperature of 6° C. Reaction is agitated for no less than 3 hours, then, mixture is brought to ambient temperature, agitation is stopped, layers are settled and separated. To the organic layer, DCM (4.0 V) and brine are added (2.0 V). The layers are separated, and organics are filtered through a charcoal filter. The obtained solution is subjected to azeotropic distillations at atmospheric pressure to remove water targeting final volume of 8 V. The obtained slurry is brought to ambient temperature and filtered. Solids are washed with IPA (2.0V) and dried in vacuum to obtain compound (2a′) in 69% yield.

### Analytical Information

compound (2a′): ¹H NMR (400 MHZ, DMSO-d₆) δ ppm-1.33-1.51 (m, 2H) 1.89 (br dd, J=12.76, 3.00 Hz, 2H) 2.26 (s, 3H) 2.58 (t, J=6.38 Hz, 2H)-3.20-3.32 (m, 2H)-3.76-3.87 (m, 5H)-3.88-4.02 (m, 1H) 4.83 (br signal, 2H) 6.89 (d, J-8.76 Hz, 1H) 7.00 (dd, J=8.76, 2.25 Hz, 1H) 7.10 (d, J=2.25 Hz, 1H) 7.57 (d, J=8.76 Hz, 1H) 7.77 (dd, J=8.76, 2.50 Hz, 1H) 7.84 (d, J=2.50 Hz, 1H) 8.04 (d, J=7.50 Hz, 1H) 8.17 (s, 1H) 8.39 (s, 1H) 9.07 (s, 1H) 9.58 (s, 1H); LR EI MS m/z: 572.17

Step 6. Preparation of Form I of Compound (1) from Compound (2a′).

### General Procedure.

All calculations are made with respect to compound (2a′).

To a clean N₂-sparged Vessel, compound (2a′) (1.0 equiv.) and THF (8.0 V) are charged and agitation is started. To this mixture, a solution of KOH (2.0 equiv.) in water (3.5 V) is charged while maintaining internal temperature of vessel of 23° C. Reaction is heated to 38° C. and agitated for no less than 18 hours. Upon completion of reaction, mixture is brought to ambient temperature, agitation is stopped, and aqueous layer is removed. Organics are concentrated under vacuum to target volume of 4.0 V and diluted with DCM (6.0 V) and MeOH (3.0 V). Resulting solution is washed with brine (2.0 V) and organics are separated. DCM and MeOH are exchanged with THE via atmospheric distillation to target volume of 8.0 V. Mixture is brought to ambient temperature and slurry is filtered. Solids are washed with THF (2.0 V) and dried in vacuum to yield crystalline form I of compound (1) in 91% yield.

### Step 7. Conversion of Form I of Compound (1) to Form III of Compound (1).

### General Procedure.

All calculations are made with respect to form I of compound (1).

To a clean N₂-sparged Vessel, crude form I of compound (1) (1.0 equiv.), iso-propanol or iso-propyl acetate (8.0 V) are charged and agitation is started. If available, seeds of form III of compound (1) can be charged. Slurry is agitated at ambient temperature for 2 hours and filtered. Solids are washed with iso-Propanol or isopropyl acetate (2.0 V) and dried in vacuum to yield form III of compound (1) in 97% yield.

### Example 1.2—Route 2 to Obtain Crystalline Forms I and IV

Scheme 2. Synthesis of Compound (1) from Compound (9), Compound (10), and Compound (7′), Based on a Sulfur Comprising Leaving Group

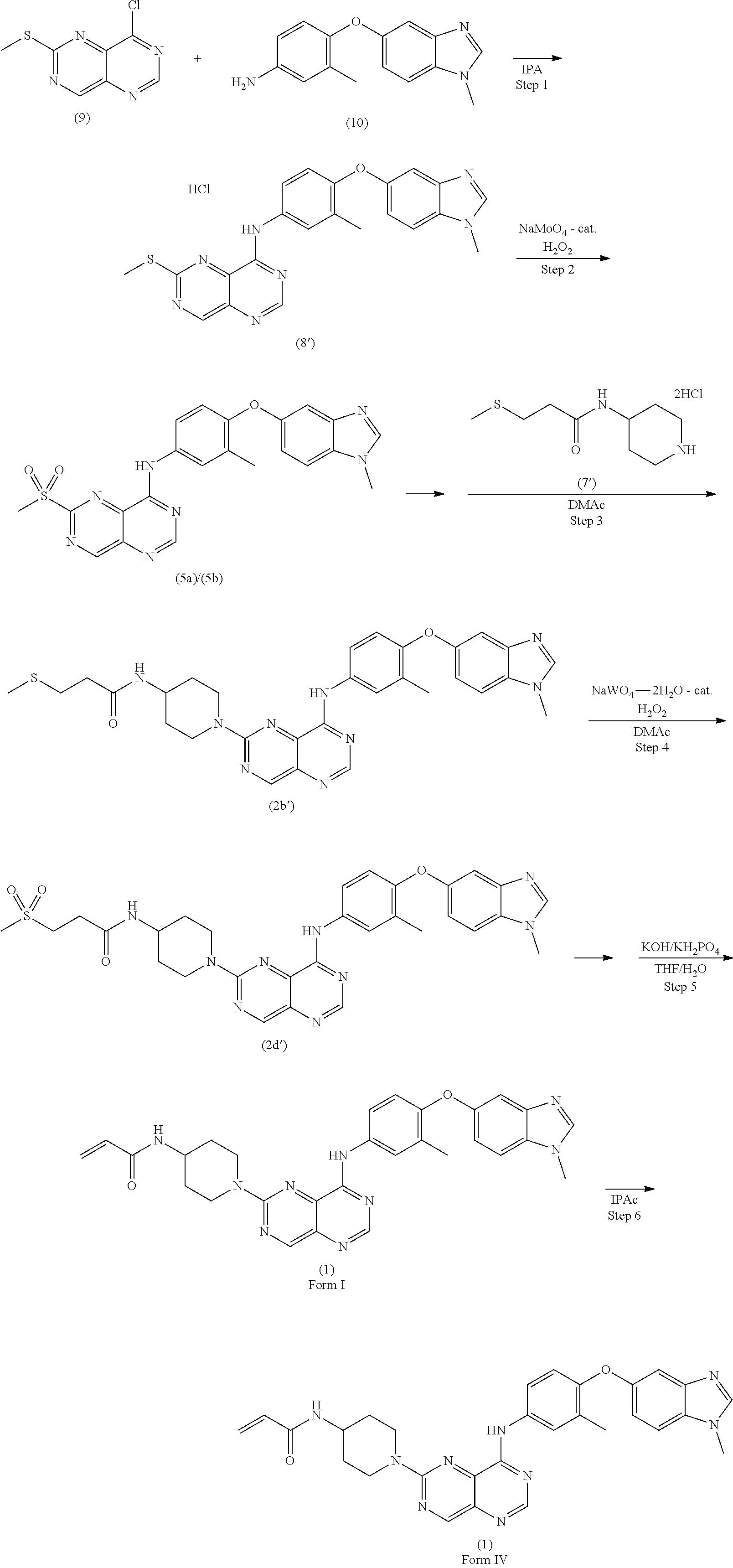

### Steps 1 and 2 are Performed as Described in Example 1.1.

Step 3. Preparation of Compound (2b′) from Compound (5a)/(5b) and Compound (7′).

### General Procedure.

All calculations are made with respect to compound (5a).

To a clean N₂-sparged Vessel #1, compound (7′) (1.4 equiv.), water (1.0 V), brine (0.75 V), and 2-Me-THF (9.0 V) are charged and agitation is started. Mixture is cooled to 10° C. and 50% aqueous NaOH solution (0.47 V) is added while maintaining internal temperature of vessel below 20° C. Biphasic mixture is vigorously agitated. Agitation is stopped after layers form or after approximately 30 to 60 minutes, layers are settled and separated. Aqueous layer is back-extracted with 2-Me-THF (4V). Then, organics are combined and 2-Me-THF is exchanged with DMAc to target volume of ˜7 V. Solution of free base of compound (7′) in DMAc thus obtained is stored until used in the next steps.

To a clean N₂-sparged Vessel #2, compound (5a)/compound (5b) (1.0 equiv.) is charged followed by addition of compound (7′) solution in DMAc. Agitation is started, mixture is heated to 68° C. and held for approximately 6 hours. Then, mixture is brought to ambient temperature and filtered. Solids are washed with MeOH (4 V) and dried in vacuum to yield compound (2b′) as a solid in 76% yield.

### Analytical Information

compound (2b′): ¹H NMR (400 MHZ, DMSO-d₆) δ ppm 1.33-1.49 (m, 1H) 1.83-1.95 (m, 1H) 2.06 (s, 1H) 2.26 (s, 1H) 2.37 (t, J=7.25 Hz, 1H) 2.67 (t, J=7.25 Hz, 1H) 3.26 (brt, J=11.51 Hz, 1H) 3.84 (s, 1H) 3.88-3.98 (m, 1H) 4.75-4.90 (m, 1H) 6.89 (d, J=8.75 Hz, 1H) 7.00 (dd, J=8.50, 2.00 Hz, 1H) 7.10 (d, J=2.00 Hz, 1H) 7.57 (d, J-8.76 Hz, 1H) 7.77 (dd, J-8.76, 2.25 Hz, 1H) 7.84 (d, J=2.00 Hz, 1H) 7.91 (br d, J=7.50 Hz, 1H) 8.17 (s, 1H) 8.39 (s, 1H) 9.07 (s, 1H) 9.57 (s, 1H); LR EI MS m/z: 292.74.

Step 4. Preparation of Compound (2d′) from Compound (2b′).

### General Procedure.

All calculations are made with respect to compound (2b′).

To a clean N₂-sparged Vessel #1, compound (2b′) (1.0 equiv.), DMAc (8.0 V), and a solution of Na₂WO₄ (2 mol %) in water (0.45 V) are charged. Agitation is started and mixture is heated to 70° C. 30% aqueous solution of H₂O₂ (2.6 equiv.) was charged slowly while maintaining internal temperature of vessel of 70° C. Upon completion of addition, mixture is agitated at 70° C. for approximately 16 hours, then, cooled to 50° C. and DMSO (0.22 equiv.) is added. Mixture is further cooled to 4° C. followed by addition of IPAc (4.0 V). The obtained slurry is agitated for an additional 4 hrs at 4° C. and filtered. Solids are washed with a mixture of DMAc (0.76 V) and IPAc (2.25V) and dried in vacuum. Obtained compound (2d′) is recharged into the vessel and triturated in water (16V) at 72° C. for 18 hours. Then, slurry is cooled, product is filtered and dried in vacuum.

If needed, compound (2c′) can be enriched in reaction mixture with employment of sub-stoichiometric amount of oxidant and then isolated by standard purification methods. Compound (2c′) is characterized below on 300 MHz NMR spectrometer and infusion MS.

### Analytical Information

compound (2d′): ¹H NMR (400 MHZ, DMSO-d₆) δ ppm-1.34-1.48 (m, 2H)-1.85-1.95 (m, 2H)-2.54-2.62 (m, 2H) 2.99 (s, 3H)-3.22-3.38 (m, 4H) 3.84 (s, 3H)-3.87-4.00 (m, 1H) 4.82 (br d, 2H) 6.89 (d, J=8.75 Hz, 1H) 7.00 (dd, J=8.76, 2.25 Hz, 1H) 7.10 (d, J=2.25 Hz, 1H) 7.57 (d, J=8.50 Hz, 1H) 7.77 (dd, J=8.76, 2.75 Hz, 1H) 7.84 (d, J=2.50 Hz, 1H) 8.10 (d, J=7.75 Hz, 1H) 8.17 (s, 1H) 8.39 (s, 1H) 9.06 (s, 1H) 9.56 (s, 1H); LR EI MS m/z: 616.22. compound (2c′): 1H NMR (300 MHz, DMSO-d₆) δ ppm-1.34-1.58 (m, 2H)-1.83-2.02 (m, 2H) 2.27 (s, 3H)-2.50-2.66 (m, 4H)-2.77-3.41 (m, 6H) 3.84 (s, 3H)-3.88-4.05 (m, 1H) 4.77 (br d, J=13.33 Hz, 1H) 6.90 (d, J-8.65 Hz, 1H) 6.99 (dd, J=8.72, 2.27 Hz, 1H) 7.13 (d, J-2.20 Hz, 1H) 7.53 (d, J=8.79 Hz, 1H)-7.73-7.85 (m, 2H) 7.94 (d, J=7.47 Hz, 1H) 8.13 (s, 1H) 8.38 (s, 1H) 9.02 (s, 1H) 9.37 (s, 1H): LR MS: 600.25.

Step 5. Preparation of Form I of Compound (1) from Compound (2d′).

### General Procedure.

All calculations are made with respect to compound (2d′).

To a clean N₂-sparged Vessel #1, compound (2d′) (1.0 equiv.), 45% aqueous KOH solution (1.5 equiv.), THF (8.9 V), and MeCN (1.6 V) are charged and agitation is started. Mixture is heated to 42° C. and agitated for ˜14 hrs followed by cooling to 30° C. and agitation for an additional 10 hrs. Upon completion of reaction, mixture is reheated to 45° C., agitations is stopped, and layers are separated. The organic layer is consecutively washed with 2.5 M Phosphate Buffer and brine. Organics are subjected to azeotropic distillation using THF to remove water targeting final 6 V volume. Upon completion of distillation, slurry is brought to ambient temperature, filtered and washed with THF. Solids are dried in vacuum at 50° C. to yield crystalline form I of compound (1) in 81% yield.

### Step 6. Conversion of Form I of Compound (1) to Form IV of Compound (1).

### General Procedure.

All calculations are made with respect to compound (1).

To a clean N₂-sparged Vessel, compound (1) (1.0 equiv.), seeds of form IV of compound (1) (2 wt %) and IPAc (20V) are charged, and agitation is started. Mixture is heated to 70° C. and agitated for ˜20 hrs followed by cooling to 20° C. Slurry is filtered and solids consecutively washed with IPAc and water. Solids are dried in vacuum at 50° C. to yield form IV of compound (1) in 95% yield.

### Example 2—Manufacture of Solid State Forms of Compound (1)

It should be noted that the input form of compound (1) is not strictly consequential for the crystallization procedures if full dissolution is achieved prior to crystallization. In case of full dissolution, the compound (1) starting material can for example be produced according to the synthesis described in WO 2021/213800 or according to the procedures disclosed under Example 1 herein.

### Example 2.1—Manufacture of Form I

First example procedure for the preparation of form I (crystallization): 19 kg of compound (1) (any solid state form) are dissolved in a mixture of ˜54 kg THF, ˜160 kg DCM and ˜48 kg MeOH. Residual inorganic salts are removed by washing with brine (48 kg). Undissolved particulates are removed by polish filtration of the organic layer. The organic layer is then distilled to ˜160 L and the mixture is diluted with 78 kg of THF. The distillation, THF dilution, distillation sequence is repeated until levels of water and MeOH≤1.0% w/w each. Upon completion of distillations, the obtained slurry is held at ambient temperature for not longer than 12 hrs and filtered to yield form I.

Second example procedure for the preparation of form I (crystallization): 6 g of form IV or any other solid form of compound (1) (e.g. prepared according to one of the Examples described herein) are dissolved in 75 g 5% w/w H₂O in IPA solution at 90° C. Solution is slowly cooled to 75° C. and seeded with 60 mg of form I. Mixture is agitated at 75° C. for 2 hours followed by cooling at 0.3° C./min rate to 20° C. Upon completion of cooling, solids are filtered and dried to yield form I.

### Example 2.2—Manufacture of Form III

Example procedure for the preparation of form III (slurry): 17 kg of form I of compound (1) are mixed with 271 kg of IPAc. Slurry is heated to 70° C. To the slurry, 0.2 kg of seeds of form III of compound (1) (e.g. prepared according to one of the Examples described herein) are added, and mixture is agitated for ˜16 hrs. Upon completion of hold, mixture is gradually cooled to 53° C. in ˜40 mins, then to 33° C. in ˜40 mins, then to 25° C. Obtained slurry is agitated for ˜1 hr and filtered. Solids are washed with 27 kg of IPAc and dried to yield form III.

The procedure can also be performed without addition of seeds.

### Example 2.3—Manufacture of Form IV

First example procedure for the preparation of form IV (crystallization): 300 mg form I of compound (1) are dispersed in 3 ml of 1-BuOH. Mixture is heated to 90° C. with over-head agitation. Dissolution can be observed. Solution is cooled with the rate of 0.2° C./min to 75° C. followed by quick cooling to 20° C. Obtained slurry is held for ˜12 hrs at 20° C. while agitated and filtered to yield form IV.

Second example procedure for the preparation of form IV (crystallization): Form III or any other solid form of compound (1) is dissolved in 10 volumes of 1-BuOH/Anisole (1:1) mixture at 110° C. Solution is subjected to distillation with slight vacuum during which most of the 1-BuOH was removed. The solution is seeded with form III seeds, held at 110° C. and a slurry is obtained. Mixture is cooled to ambient temperature while agitated and filtered to yield isolated compound (1) as form IV. This despite being seeded with form III.

Third example procedure for the preparation of form IV (slurry); A slurry of form I and IV of compound (1) is slurried in IPAc at temperatures ranging from 25-75° C. for 72-168 hrs. Mixture is brought to ambient temperature if applicable and filtered to yield compound (1) as form IV. When compound (1) is obtained as described in Example 1.2 above, this third example procedure for the preparation of form IV can also be performed starting from Form I alone, with or without seeding.

### Example 2.4—Manufacture of Reference Form II

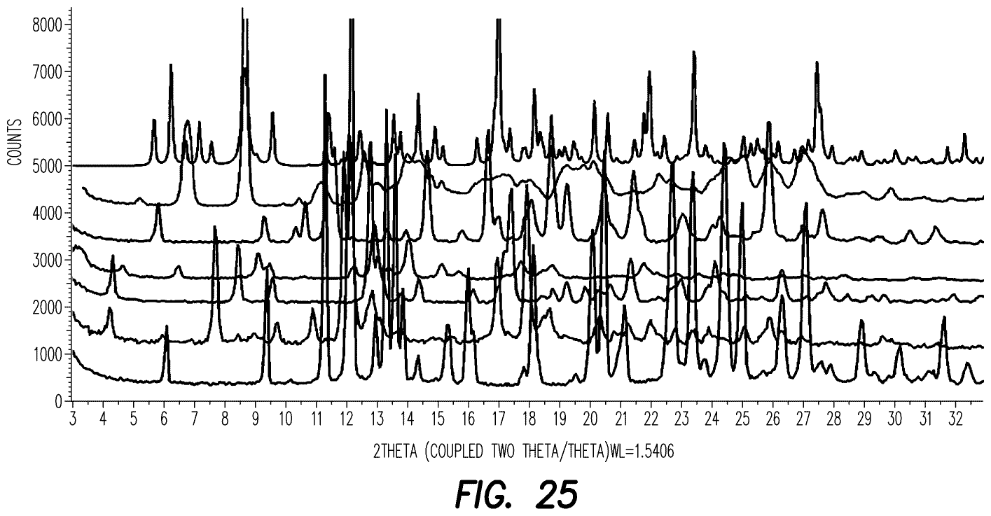

Example procedure for the preparation of form II (slurry): form I of compound (1) (30 mg) is slurried in methanol (1.5 mL) at temperature of 55° C. for 2 hrs. The mixture is cooled to room temperature and filtered to yield compound (1) as form II. The XRPD diffractogram of form II among others is shown in FIG. 25.

### Example 2.5—Manufacture of Reference Form VI

Example procedure for the preparation of form VI (slurry): form III of compound (1) is slurried in water at 50° C. for 2 hrs, cooled to room temperature and slurried for additional 168 hrs. The mixture is filtered to yield compound (1) as form VI. The XRPD diffractogram of form VI among others is shown in FIG. 25.

### Example 2.6—Manufacture of Reference Form VII

Example procedure for the preparation of form VII (crystallization): A mixture of compound (1) (30 mg) in methylethyl ketone (1.5 mL) is heated to 55° C. for 30-60 min. The obtained clear solution is filtered into a clean vial and left at room temperature without agitation to yield form VII. The XRPD diffractogram of form VII among others is shown in FIG. 25.

### Example 3—Characterisation of Solid State Forms of Compound (1)

Solid crystalline forms I, III and IV of compound (1) can be characterised by Powder X-Ray Diffraction (XRPD or PXRD), Raman spectroscopy, Differential Scanning calorimetry (DSC), Thermogravimetric Analysis (TGA),Dynamic Vapor Sorption (DVS), Scanning Electron Microscopy (EMS) and, if applicable Single Crystal X-ray diffraction (SXRD), e.g. as described below.

### Example 3.1—Characterisation by Powder X-Ray Diffraction (XRPD or PXRD)

Solid state forms of compound (1) were analysed and may be identified by XRPD. Generally, XRPD are measured at a temperature in the range of from 20 to 30° C. using CuK_{α} radiation having a wavelength of 1.54184 Å (CuKα1,2 radiation). Kα2 is removed by a software leading to CuK_{α} radiation having a wavelength of 1.54060 Å(CuKα1 radiation).

For the XRPD analysis, the methodology can be as follows.

The respective solid compound (approximately 0.2 g) is representatively subsampled into a stainless-steel sample holder fitted with a zero-diffraction plate (ZDP). The sample holder is then levelled off with a glass slide to create a flat sample surface level with the sample holder. The instrument used for the analysis is a Bruker D2 Phaser (system EQ-SSRD-XRD-01). A corundum reference standard is run each day to evaluate the system performance. Two peaks must be within +0.02° 20 for system suitability to be acceptable. The instrument settings for the measurement of the solid compound samples can be seen in Table 1. Processing (Kα2 contribution stripping, peak labelling) is completed using DIFFRAC.EVA software (version 5.0). In the following, experimental parameters for XRPD measurements are given:

TABLE 1
experimental parameters for XRPD measurements

| | | Regions of interest |
|---|---|---|
| Parameter | Full range scan | scans |
| Model | Bruker D2 Phaser | Bruker D2 Phaser |
| X-ray radiation source | Cu Kα (λ = | Cu Kα (λ = |
| | 1.54184 Å) | 1.54184 Å) |
| Current (mA) | 10 | 10 |
| Voltage (kV) | 30 | 30 |
| Scan type | Coupled Two Theta/ | Coupled Two Theta/ |
| | Theta | Theta |
| Scan mode | Continuous PSD fast | Continuous PSD fast |
| Application type | Powder Diffraction | Powder Diffraction |
| Goniometer radius (mm) | 141.0 | 141.0 |
| Range (°2θ) | 5 to 30 | 6.75 to 8.75, 11.5 to |
| | | 13.0, 13.75 to 15.25, |
| | | 18.0 to 19.75 |
| PSD opening (°) | 2.5 | 3.0 |
| Step size (°) | 0.01 | 0.014 |
| Rate (s/step) | 1.0 | 10.0 |
| Divergence slit width | 0.6 | 0.6 |
| (mm) | | |
| Detector slit width (mm) | 3 | 3 |
| Air scatter screen (mm) | 1 | 1 |
| Rotation | 15 | 15 |
| (rotations/minute) | | |

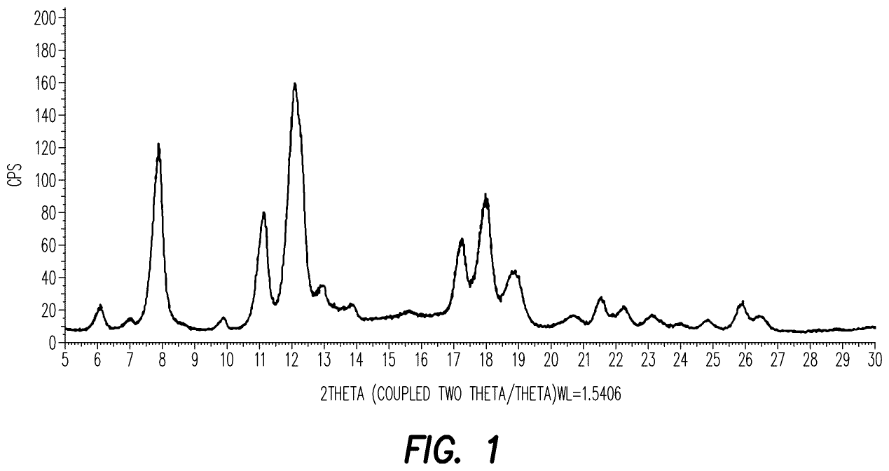

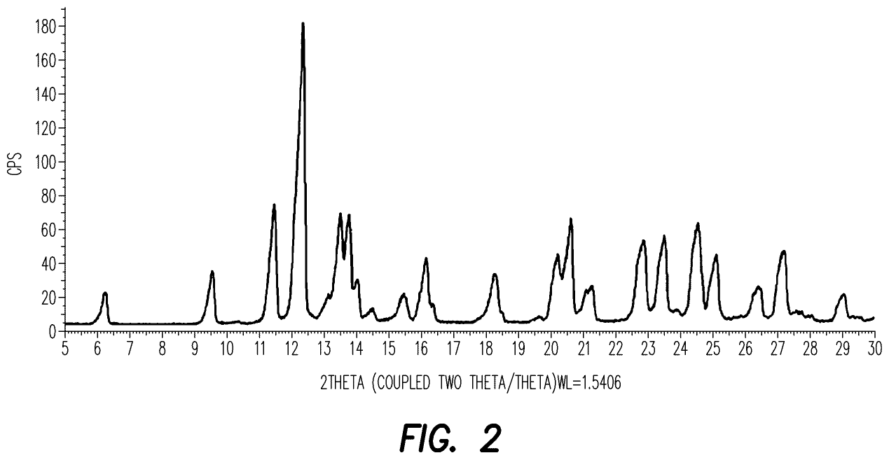

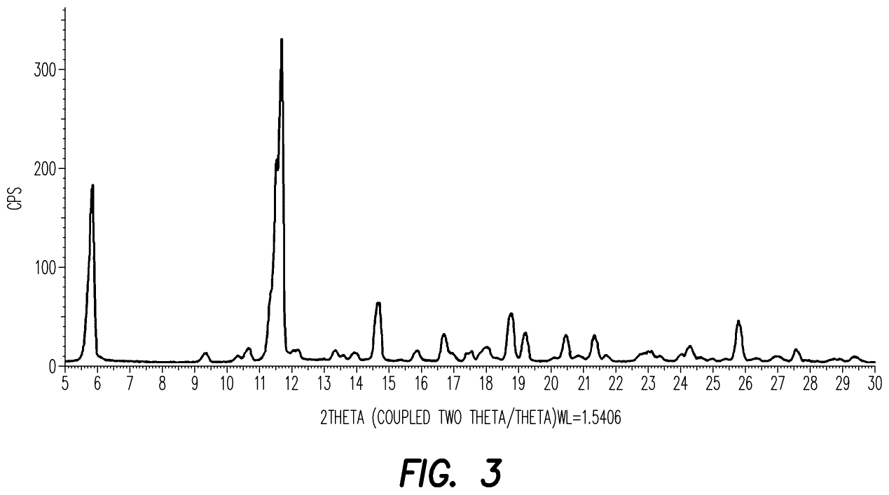

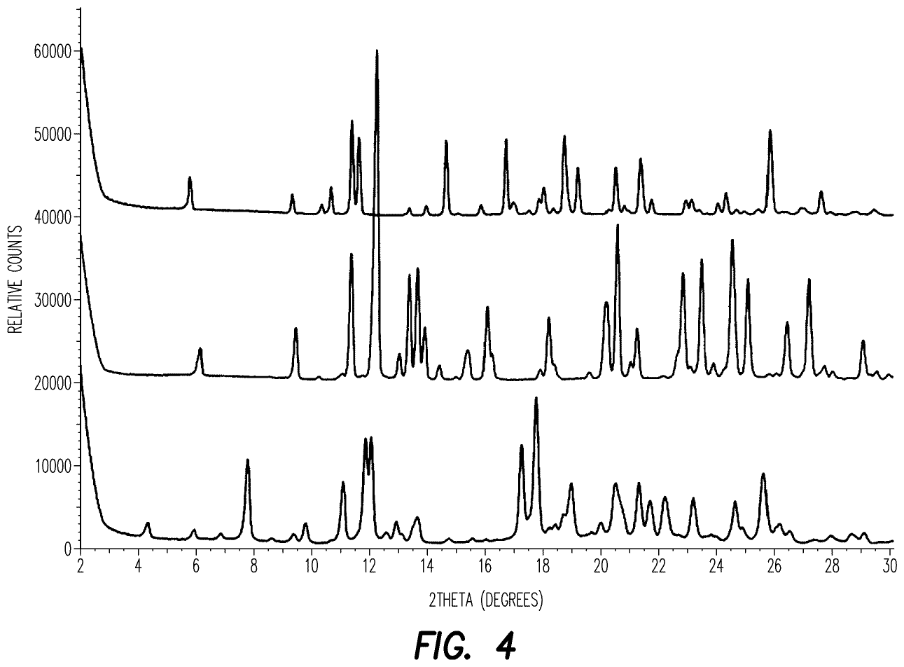

The processed XRPD diffractogram for each of the polymorphic forms are shown in FIG. 1 (form I), FIG. 2 (form III) and FIG. 3 (form IV). An XRPD overlay of all three forms is displayed in FIG. 4. It can be seen that each of the three polymorphic forms has its own unique XRPD fingerprint, and each form is identifiable by XRPD. Table 2 lists the peaks for each form (that are above 5% relative intensity). Table 3 lists the best characteristic peaks to use when trying to identify a given polymorphic form where another form is present. The diagnostic peaks are indicative of peak positions where the impurity has a relatively high intensity peak, and the sample dominant form has a flat baseline.

TABLE 2
XRPD peak comparison

| Form I (°2θ) | Form III (°2θ) | Form IV (°2θ) |
|---|---|---|
| 6.063# | — | 5.872**, # |
| — | 6.224*, # | — |
| 7.853**, # | — | — |
| — | 9.535* | — |
| 11.095*, # | — | — |
| — | 11.448*, # | 11.538** |
| — | — | 11.690**, # |
| — | — | — |
| 12.048**, # | 12.352**, # | — |
| 12.923*, # | — | — |
| — | 13.138 | — |
| — | 13.504* | — |
| 13.875 | 13.776* | — |
| — | 14.011* | — |
| — | — | 14.670*, # |
| 15.602 | 15.449 | — |
| — | 16.151*, # | — |
| — | 16.344 | — |
| — | — | 16.706, # |
| 17.219*, # | — | — |
| 17.943**, # | — | — |
| — | 18.273*, # | — |
| 18.849*, # | — | 18.778*, # |
| — | — | 19.202, # |
| — | 20.190* | |
| — | 20.621* | 20.463 |
| — | 21.079 | — |
| — | 21.267* | 21.349 |
| 21.521* | — | — |
| 22.213* | — | — |
| — | 22.852* | — |
| 23.090 | — | — |
| — | 23.492* | — |
| — | — | 24.300, # |
| — | 24.540* | — |
| — | 25.126* | — |
| 25.891*, # | — | 25.796*, # |
| 26.491 | 26.417* | — |
| — | 27.210*, # | — |
| — | 29.068 | — |

With regard to Table 2, peaks shown with a #are determined to be characteristic peaks, peaks marked “*” have a greater than 10% relative intensity, peaks marked “**” have a greater than 50% relative intensity. Further, the peaks are listed in order of peak position (° 2θ), with similar peak positions on the same row.

TABLE 3
XRPD characteristic peaks (°2θ) for
polymorphic impurity identification

Form to be

| detected | Sample matrix | | |
|---|---|---|---|
| as impurity | In Form I | In Form III | In Form IV |
| Form I | — | 7.85, 17.22, | 7.85, 11.10, |
| | | 25.89 | 12.05 |
| Form III | 27.21, 16.15, 11.45 | — | 18.27, 6.22 |
| Form IV | 14.67, 11.69, 16.71 | 5.87, 18.78, | — |
| | | 19.20, 25.80 | |

### Example 3.2—Characterisation by Raman Spectroscopy

The produced crystalline forms I, III, and IV of compound (1) were further analyzed and may be identified by means of Raman spectroscopy. The methodology for this can be as follows. Solid compounds are representatively subsampled into a vial lid to approximately 0.5 mm thickness or greater. The instrument used for the analysis is a BWTek i-Raman Plus probe. The instrument settings for the measurement of the solid compound samples can be seen in Table 4. Processing (dark subtraction, background removal, peak labelling, diffractogram figure creation) is completed using BWSpec 4 software. The experimental parameters are further detailed in following Table 4:

TABLE 4
Raman measurement experimental parameters

| Parameter | Value | |
|---|---|---|
| Model | BWTek i-Raman Plus | |
| Laser excitation | 785 | nm |
| wavelength | | |
| Spectral range | 150 to 4000 cm⁻¹ | |
| Number of averages | 3 | |
| Total integration time | 10 | s |

| | |
|---|---|
| Laser power level | Varied to achieve maximum signal |
| | without oversaturation |

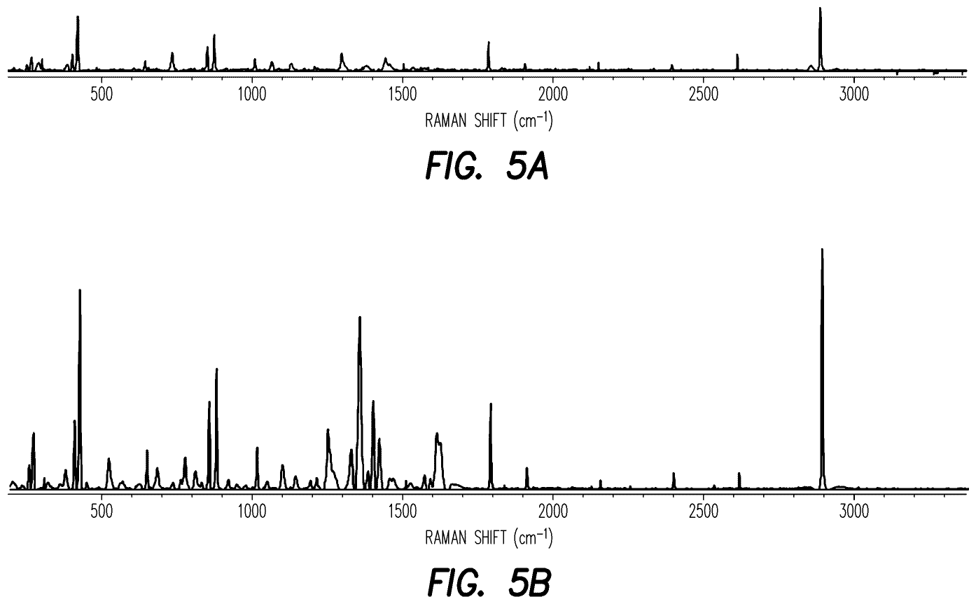

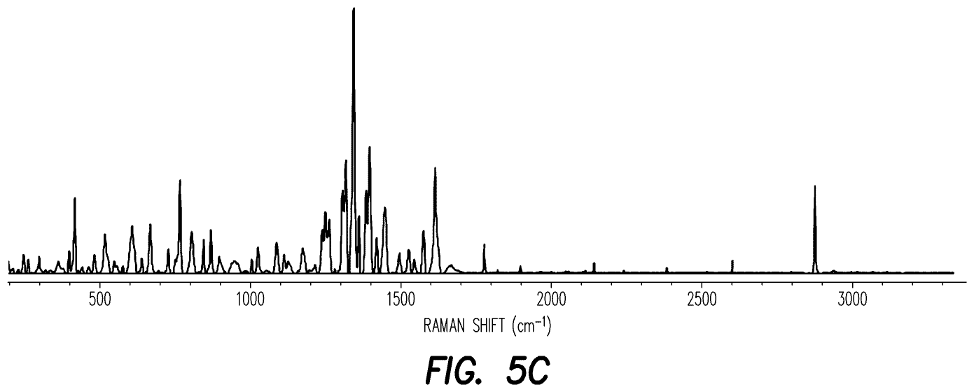

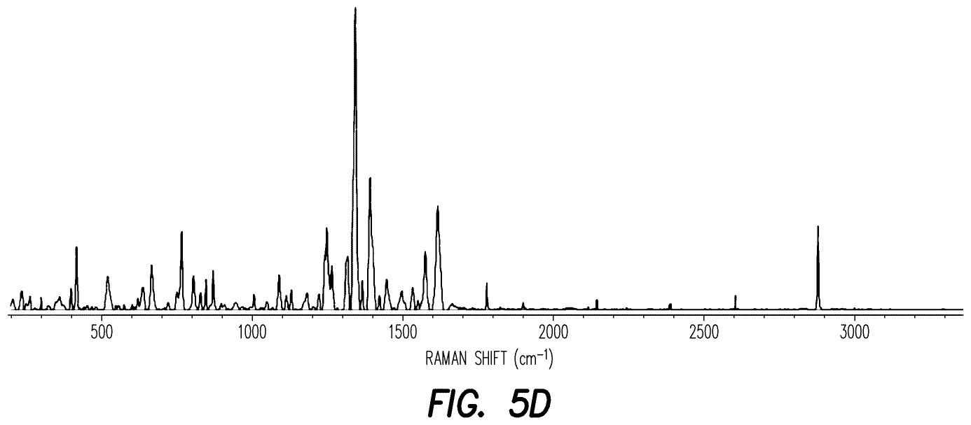

The processed Raman spectra can be seen individually in FIGS. 5a to 5d, wherein FIG. 5a shows background Raman spectra; FIG. 5b shows a Raman spectrum of crystalline form I; FIG. 5c shows a Raman spectrum of crystalline form III; and FIG. 5d shows a Raman spectrum of crystalline form IV. A summary of the peaks is listed in Table 5. In Table 5, only peaks not present in the background are listed, bolded peaks are determined to be characteristic peaks and the peaks are listed in order of Raman shift (cm-1), with similar shifts listed on the same row.

Further, Table 6 shows only Raman peaks (cm-1) characteristic for polymorphic form impurity identification.

TABLE 5
Raman peak comparison

| Form I (cm⁻¹) | Form III (cm⁻¹) | Form IV (cm⁻¹) |
|---|---|---|
| | | 237.7# |
| 373.0 | 365.9 | 363.8 |
| | 484.8# | |
| 514.9 | 520.0 | 522.0 |
| | 609.9# | |
| | | 639.9# |
| 676.4 | 671.2 | 668.0 |
| 754.9 | 754.1 | 752.4 |
| 768.5 | 768.7 | 767.7 |
| 803.1 | 807.6 | 806.7 |
| 823.7# | | |
| | | 831.1# |
| | 900.4 | 898.7 |
| 911.4 | | 910.5 |
| | 1028.5# | |
| 1091.0 | 1090.7 | 1091.4 |
| | 1115.6 | 1115.0 |
| 1183.5 | 1177.7 | 1184.5 |
| 1241.6 | 1243.7 | 1243.1 |
| | 1253.2 | 1250.0 |
| | 1265.4 | 1267.0 |
| | 1309.7# | |
| 1318.4 | 1321.2 | 1319.2 |
| 1345.7 | 1346.6 | 1344.4 |
| 1373.6 | 1364.8 | 1368.4 |
| 1390.5 | 1388.4 | 1393.6 |
| | 1400.4# | |
| 1411.4# | | |
| | 1422.9 | 1424.3 |
| | 1452.0 | 1448.9 |
| | 1529.0 | 1533.8 |
| 1561 | | |
| | 1578.1 | 1575.9 |
| 1602.1# | | |
| 1613.8 | 1617.1 | 1616.9 |

TABLE 6
characteristic Raman peaks (cm⁻¹) for
polymorphic form impurity identification

Form to be

| detected | Sample matrix | | |
|---|---|---|---|
| as impurity | In Form I | In Form III | In Form IV |
| Form I | — | 373, 1602, 515, | 1411, 1602, |
| | | 911 | 373, 515 |
| Form III | 610, 485, 1400 | — | 1029, 485, 610 |
| Form IV | 238, 640 | 831, 238, 640 | — |

### Example 3.3—Characterisation by Differential Scanning Calorimetry (DSC)

The produced crystalline forms I, II, and IV of compound (1) were further analyzed and may be identified by means of Differential Scanning calorimetry (DSC). The methodology for this can be as follows. The solid forms (˜5 mg) are placed in an aluminium pan and sealed. The instrument used for the analysis is a TA Instruments DSC 25. Experimental settings are listed 10 in Table 7. Processing is completed using TRIOS software.

TABLE 7
Experimental settings for DSC analysis

| Parameter | Value |
|---|---|
| Model | TA Instruments DSC 25 |
| sample pan type | Aluminum |
| Temperature range | 40° C. to 300-350° C. |
| Ramp rate | 10° C./min |

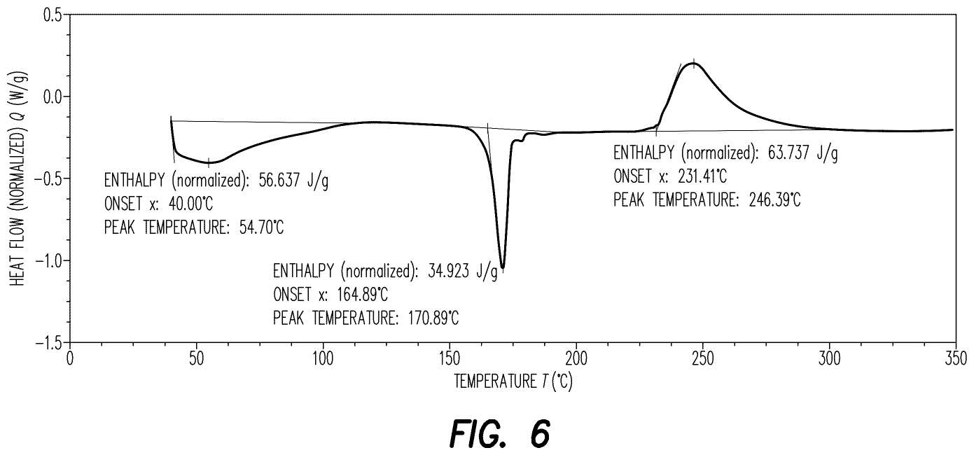

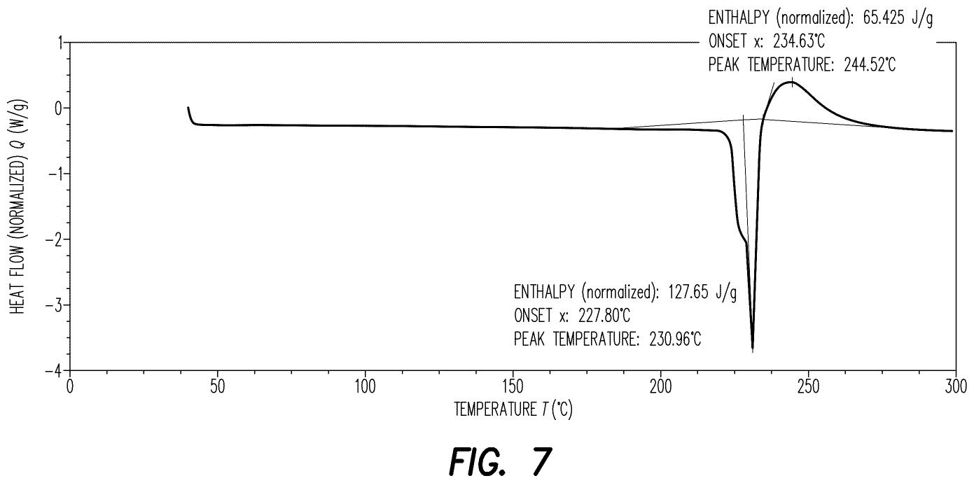

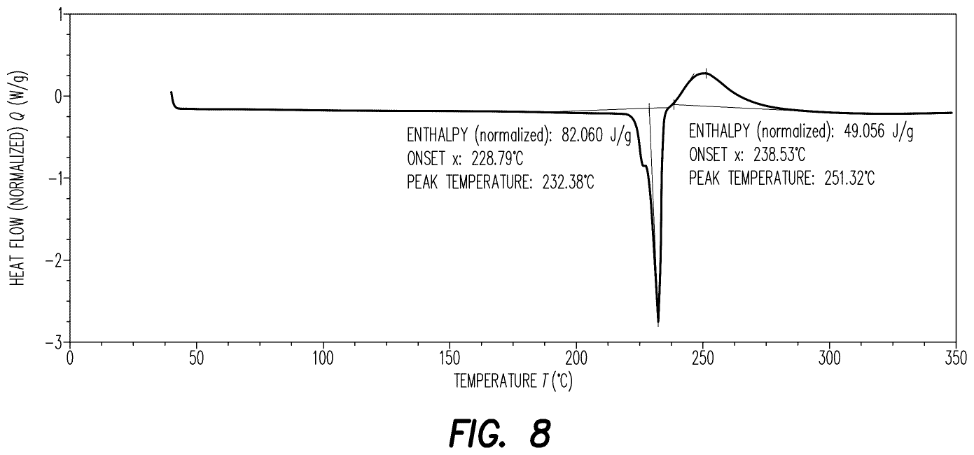

The DSC plots for each polymorphic form of compound (1) can be seen in FIG. 6 (form I) FIG. 7 (form III) and FIG. 8 (form IV).

A summary of the DSC events is listed in Table 8. From comparison of the melting points of each form, it is expected that form IV is most thermodynamically stable, very closely followed by form III. Form I is expected to be less thermodynamically stable. An exothermic event around 240° C. was observed in all three polymorphic forms I, III, and IV.

TABLE 8
DSC events

Polymorphic

| Form | Peak temperature | | Onset | | Description |
|---|---|---|---|---|---|
| Form I | 54.7° | C. | 40° | C. | Solvent loss |
| | | | | | (THF/ethanol/water) |
| | 170.89° | C. | 164.89° | C. | Melting point |
| | 246.39° | C. | 231.41° | C. | Exothermic event |
| Form III | 230.96° | C. | 227.80° | C. | Melting point |
| | 244.52° | C. | 234.63° | C. | Exothermic event |
| Form IV | 232.38° | C. | 228.79° | C. | Melting point |
| | 251.32° | C. | 238.53° | C. | Exothermic event |

### Example 3.4—Characterisation by Thermogravimetric Analysis (TGA)

The produced crystalline forms I, III, and IV of compound (1) were further analyzed and may be identified by means of Thermogravimetric Analysis (TGA). The methodology for this can be as follows. Solid compounds (˜5 mg) are placed in an aluminium pan and sealed. The instrument used for the analysis is a TA Instruments TGA 550. Experimental settings are listed in Table 9. The same sample lots from DSC testing are used for TGA testing. Processing is completed using TRIOS software.

TABLE 9
Experimental settings for TGA analysis

| Parameter | Value |
|---|---|
| Model | TA Instruments TGA 550 |
| sample pan type | Aluminum |
| Temperature range | ambient to 600° C. |
| Ramp rate | 20° C./min |

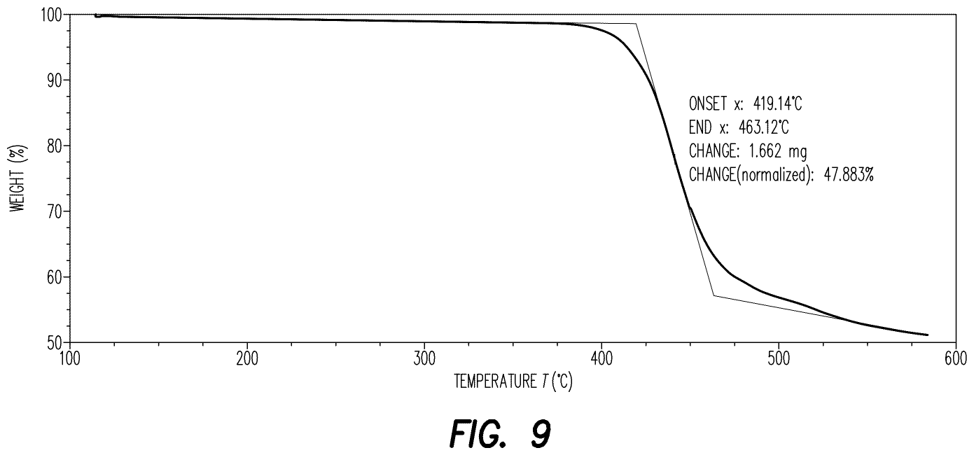

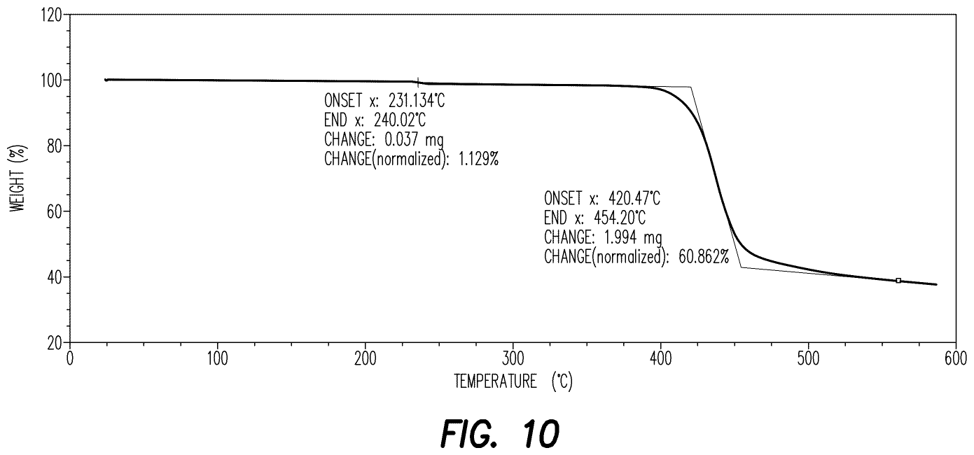

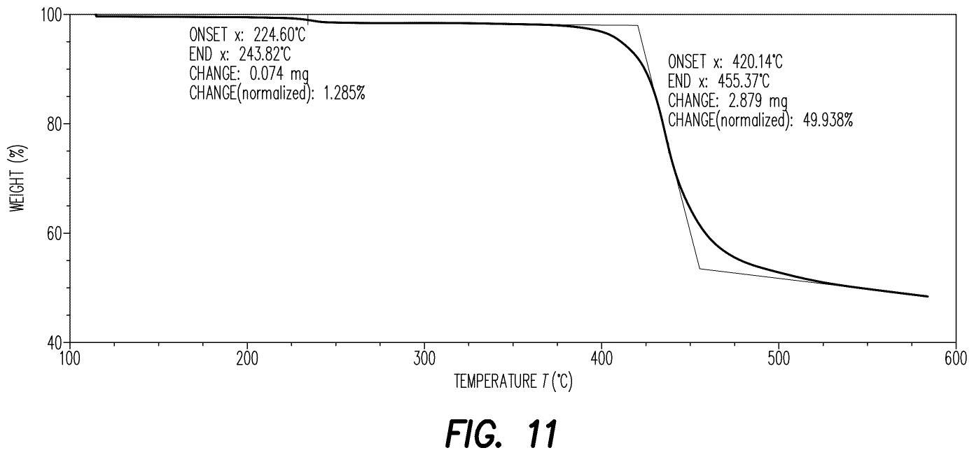

The TGA plots for each polymorphic form of compound (1) can be seen in FIG. 9 (form I) FIG. 10 (form III) and FIG. 11 (form IV). A summary of the TGA events is listed in Table 10. All three of the forms decomposed around 440° C. The event of around 1% mass loss correlates to the melting temperatures in form III and form IV, around 235° C. It is possible that this small mass loss is due to solvent being released from the crystalline lattice of form III and form IV solids upon melting.

TABLE 10
TGA events

| Polymorphic | Change in | | | |
|---|---|---|---|---|
| Form | mass | Onset | End | Description |
| Form I | 47.883% | 419.14° C. | 463.12° C. | Decomposition |
| Form III | 1.129% | 231.13° C. | 240.02° C. | Small mass loss |
| | | | | event |
| | 60.862% | 420.47° C. | 454.20° C. | Decomposition |
| Form IV | 1.285% | 224.60° C. | 243.82° C. | Small mass loss |
| | | | | event |
| | 49.938% | 420.14° C. | 455.37° C. | Decomposition |

### Example 3.5—Characterization by Dynamic Vapor Sorption (DVS)

To study the hygroscopic behaviour of crystalline forms I, III and IV of compound (1), sorption isotherms obtained by DVS were recorded with a DVS-1 or DVS intrinsic from Surface Measurement Systems. The methodology for this can be as follows. The mass of the solids is monitored as the relative humidity (RH) was deliberately changed. The RH is cycled twice from 0% to 90% to 0% in 10% steps. The temperature during measurement is kept constantly at (25.0 ±1.0° C.) Each step is held until mass stability was achieved. A camera inside the instrument captured an image at the conclusion of each stage, allowing for the observation of any visual changes in the solid form during the analysis.

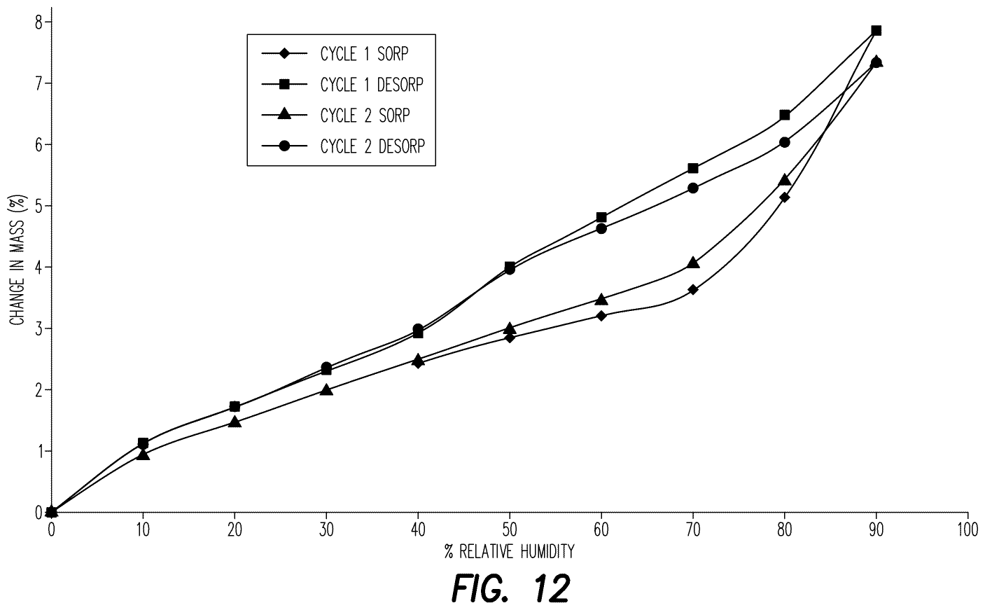

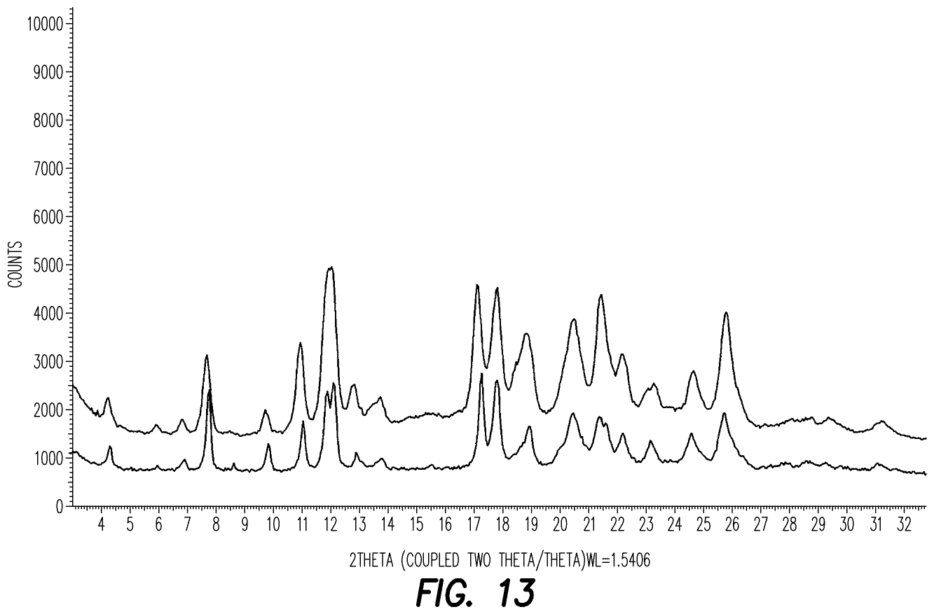

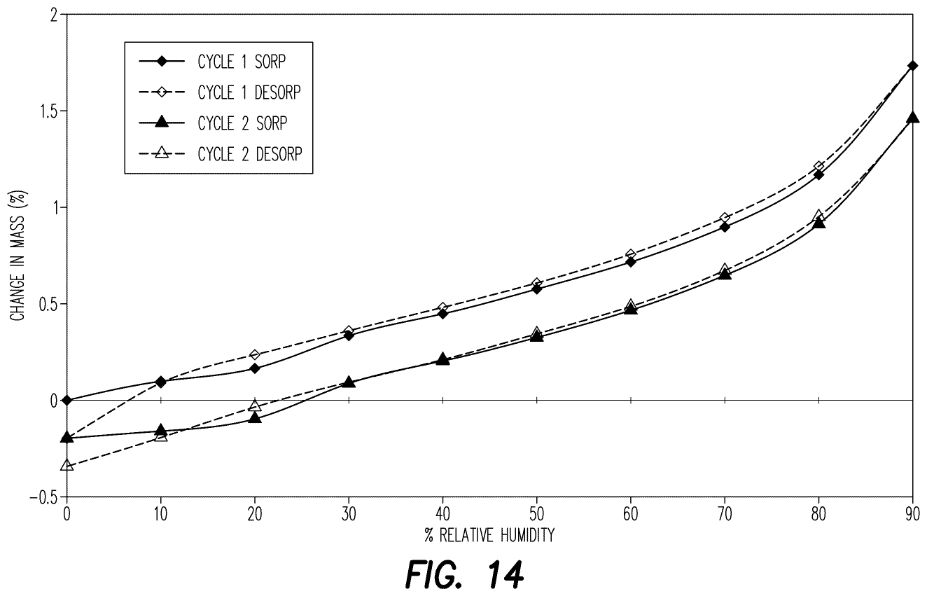

FIG. 12 shows the isotherms plot of form I. Maximum water uptake (=change in mass, Δm) of 7.3 wt % is observed up to 90% RH, which is fully reversible. The sample absorbs 5.4 wt % moisture at 80% RH. Therefore, form I is classified as hygroscopic material. Despite the significant hysteresis observed between the sorption and desorption curves, in particular in the ranges above about 40% RH, no irreversible phase change occurred during the DVS experiment as confirmed by PXRD, performed with the sample before and after the DVS run (FIG. 13). FIG. 14 shows the isotherms plot of form III. Maximum water uptake of 1.7 wt % is observed up to 90% RH, which is fully reversible. The sample absorbs 1.2 wt % moisture at 80% RH. Therefore, form III is classified as slightly hygroscopic material. There is no indication of irreversible phase conversion or hydrate formation as a result of water vapour sorption as indicated by a lack of hysteresis between the sorption and desorption curves. No change in the visual appearance was observed for form III during the humidity cycles.

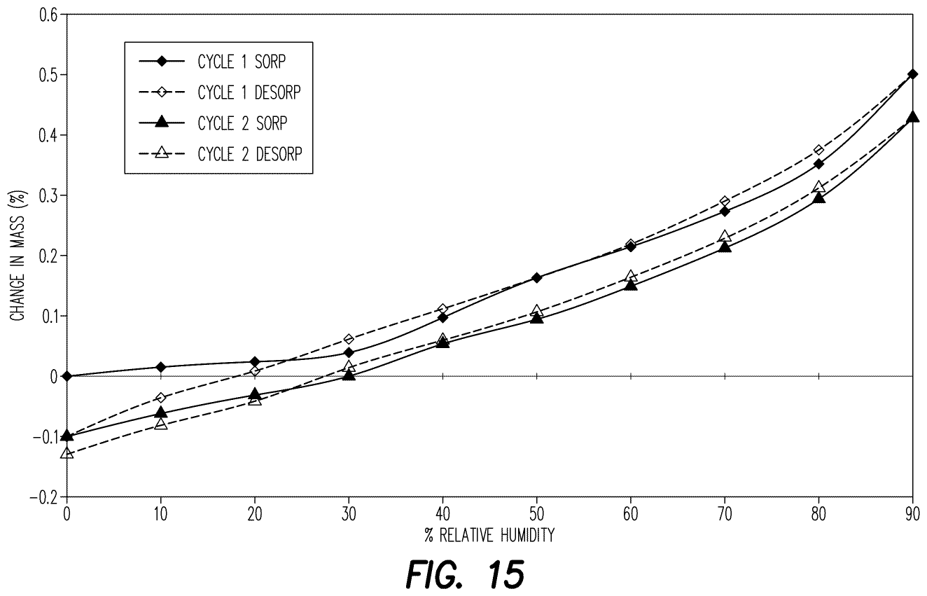

FIG. 15 shows the isotherms plot of form IV. Maximum water uptake of 0.5 wt % is observed up to 90% RH, which is fully reversible. The sample absorbs 0.4 wt % moisture at 80% RH. Therefore, form IV is classified as slightly hygroscopic material. There is no indication of irreversible phase conversion or hydrate formation as a result of water vapour sorption as indicated by a lack of hysteresis between the sorption and desorption curves. No change in the visual appearance was observed for form IV during the humidity cycles. Table 11 summarizes the events observed for the different crystalline forms of compound (1) during the DVS experiments.

TABLE 11
DVS events

| Polymorphic | Δm | Δm | | |
|---|---|---|---|---|
| Form | (0-90%) | (0-80%) | Hygroscopicity | Hysteresis |
| Form I | 7.3 wt % | 5.4 wt % | hygroscopic | yes |
| Form III | 1.7 wt % | 1.2 wt % | slightly hygroscopic | no |
| Form IV | 0.5 wt % | 0.4 wt % | slightly hygroscopic | no |

### Example 3.6—Characterisation by Scanning Electron Microscopy (SEM)

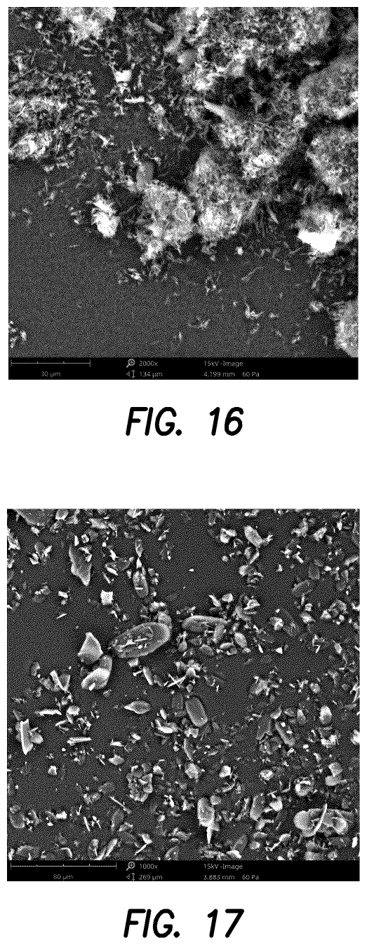

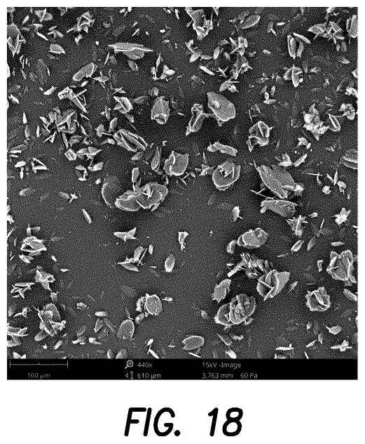

The SEM images for each of the polymorphic forms are shown in FIG. 16 (form I), FIG. 17 (form III) and FIG. 18 (form IV). While form III and form IV mainly consist of plate-like crystals, form I shows a needle like morphology.

### Example 3.7—Characterisation by Single Crystal X-Ray Diffractometry (SXRD)

Single crystals of crystalline form IV of compound (1) are grown by slow evaporation at room temperature from a solution in anisole:1-butanol (8:1). A clear and colorless chunk with approximate dimensions of 0.170×0.07×0.05 mm is selected and mounted on a MiTeGen MicroMount™ with Paratone-N oil. Three frames separated in reciprocal space are recorded to provide an orientation matrix and initial cell parameters. Final cell parameters are obtained and refined based on the full data set. Minimal diffraction was observed at typical collection times (≤60 seconds) therefore an exposure collection time of 360 seconds/degree data is employed for the full data set.

Diffraction data are acquired at room temperature on Rigaku R-AXIS RAPID diffractometer equipped with a sealed tube copper source (λ=1.54184 Å) at 50 kV/40 mA and a Spider curved image plate detector. A diffraction data set of reciprocal space is obtained to a resolution of 0.81 Å using 5° oscillation steps and 300 sec. exposure for each frame. The diffraction images are processed and scaled using Rigaku Oxford Diffraction software (RapidAuto; Rigaku OD: The Woodlands, T X, 2015). Observation of the crystal after data collection showed no signs of decomposition.

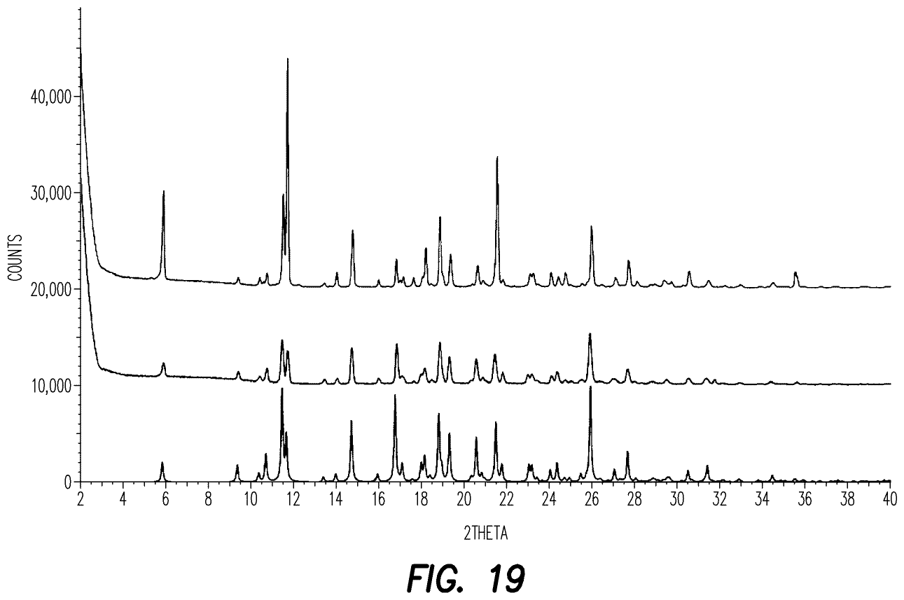

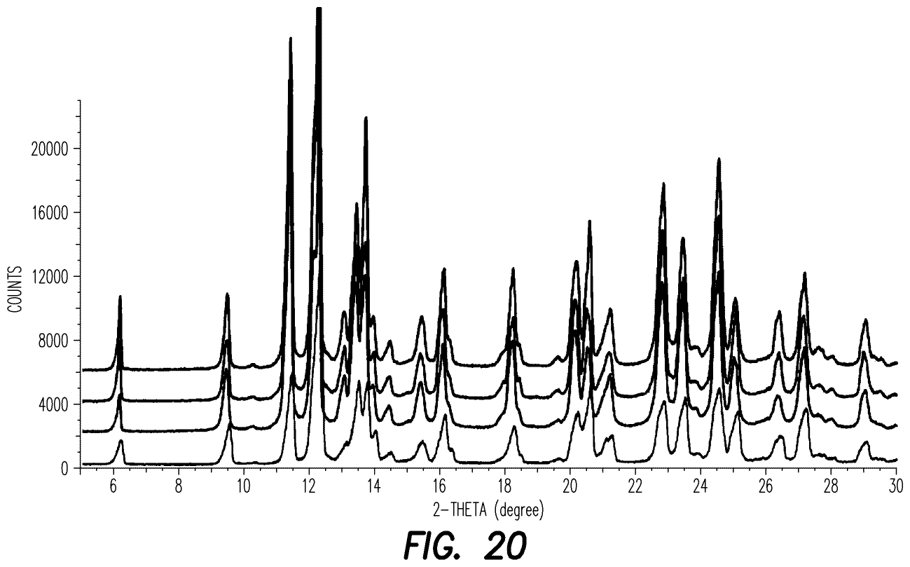

The structure is solved using Olex216 imbedded with the SHELXT17 strurogram employing Direct Methods. The structure is refined with the SHELXL18 refinement package using Least Squares minimization. All non-hydrogen atoms are refined anisotropically. Hydrogen atoms are included in the model at geometrically calculated positions and refined using a riding model. The initial structure solution provided a calculated powder diffraction pattern consistent with form IV (see bottom diffractogram of FIG. 19).

TABLE 12
Crystal data and structure refinement

| Empirical formula | C₂₉H₃₀N₉O₂ | | |
|---|---|---|---|
| Formula weight | 535.62 | | |
| Temperature | RT (283-303) K | | |
| Wavelength | 1.54184 | Å | |
| Crystal system | Triclinic | | |
| Space group | P-1 | | |
| Unit cell dimensions | a = 9.4141(12) | Å | α = 91.327(6)° |
| | b = 10.5120(13) | Å | β = 102.462(7)° |
| | c = 15.6630(19) | Å | γ = 114.978(8)° |
| Volume | 1360.5(3) | Å³ | |
| Z | 2 | | |
| Denisty (calculated) | 1.310 | Mg/m³ | |
| Absorption coefficient | 0.704 | mm⁻¹ | |

| | |
|---|---|
| F(000) | 566 |
| Crystal size | 0.050 × 0.050 × 0.030 mm³ |
| Crystal color and habit | Colorless Block |
| Diffractometer | Rigaku R-AXIS RAPID-S |
| Theta ranges for data collection | 9.36 to 136.414 |
| Index ranges | −11 <= h <= 11, −12 <= k <= 12, −18 <= I <= 18 |
| Reflections collected | 12987 |
| Independent reflections | 4800 [Rᵢₙₜ = 0.1345, R_{sigma} = 0.3480] |
| Completeness to theta = 67.684° | 99.9% |
| Absorption correction | empirical |
| Solution method | SHELXT-2014/5 (Sheldrick, 2014) |
| Refinement method | SHELXL-2014/7 (Sheldrick, 2014) |
| Data/restraints/parameters | 4800/0/485 |
| Goodness-of-fit on F² | 0.991 |
| Final R indices [I > 2sigma(I)] | R1 = 0.1166, wR2 = 0.1270 |
| R indices (all data) | R1 = 0.4023, wR2 = 0.1985 |
| Largest diff. peak and hole | 0.18 and −0.15 e · Å⁻³ |

### Example 4—Polymorphic Stability of Solid State Forms of Compound (1)

### Example 4.1—Polymorphic Stability of Form III and Form IV

5 Form III and form IV were open stored under stress conditions of 90° C./3% RH and 90° C./78% RH over a period of 21 days and analyzed by Powder X-ray diffraction for polymorphic stability.

A Bruker D2 Phaser X-Ray Diffractometer (XRD, EQ-SSRD-XRD-01) can be used for characterizing the samples. The settings used during the measurement of the sample are listed in Table 13. Raw results are evaluated using DIFFRAC.EVA (Bruker, V5.0) software. The Kα2 contribution was stripped, and a peak search was performed using peak search parameters (width 0.302 and threshold 1.0).

TABLE 13
PXRD measurement experimental parameters

| Parameter | Full range scan |
|---|---|
| Model | Bruker D2 Phaser |
| X-ray radiation source | Cu Kα (λ = 1.54184 Å) |
| Current (mA) | 10 |
| Voltage (kV) | 30 |
| Scan type | Coupled Two Theta/Theta |
| Scan mode | Continuous PSD fast |
| Application type | Powder Diffraction |
| Goniometer radius (mm) | 141.0 |
| Range (°2θ) | 5 to 30 |
| PSD opening (°) | 2.5 |
| Step size (°) | 0.01 |
| Rate (s/step) | 3.0 |
| Divergence slit width (mm) | 0.2 |
| Detector slit width (mm) | 3 |
| Air scatter screen (mm) | 1 |
| Rotation (rotations/minute) | 15 |
| Reason for use | High resolution |

A test sample (approximately 0.3 g) is representatively subsampled into an agate-type mortar and pestle. Sample is ground for approximately one minute to achieve a fine homogenous powder. Sample is then transferred into a stainless-steel sample holder fitted with a zero-diffraction plate (ZDP). The sample holder is then levelled off with a glass slide to create a flat sample surface level with the sample holder.

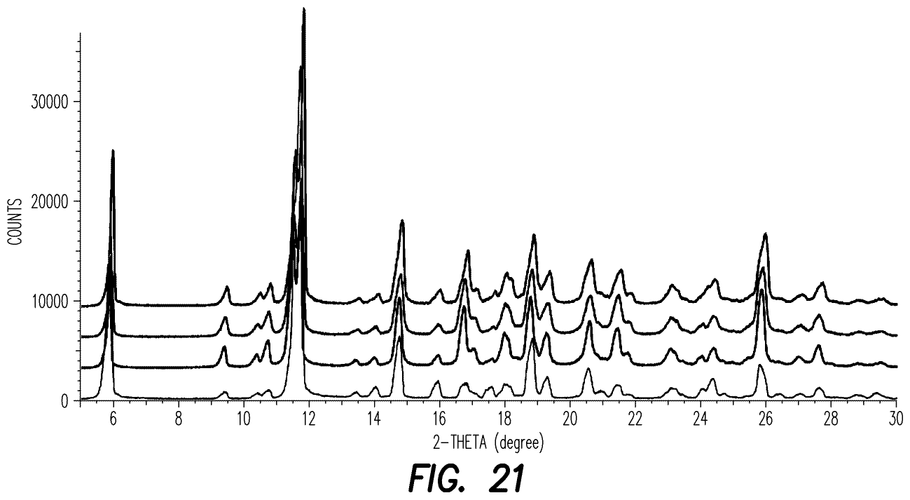

An overlay of the processed powder x-ray diffractograms for form III and form IV under different stress conditions are shown in FIG. 20 and FIG. 21, respectively. Form III control and test samples stored under 90° C./3% relative humidity (RH) and 90° C./78% RH for a period of 21 days were stable and conformed to form III reference standard, with no missing or extra reflections observed. Similarly, control and test samples of form IV stored under 90° C./3% RH and 90° C./78% RH for a period of 21 days were stable and conformed to form IV reference standard.

### Example 4.2—Polymorphic Stability of Form I, Form II and Form VI

Crystalline forms I, II and VI of compound (1) were exposed to temperature stress under conditions as outlined in Table 14, and the samples were analyzed by the powder X-ray diffraction method as described in Example 4.1 for polymorphic stability.

TABLE 14
Summary of temperature stress tests
with form I, form II and form VI

| Initial | | PXRD of |
|---|---|---|
| crystalline | | stressed |
| form | Storage conditions | sample |
| Form I | 6 h at 150° C. (closed vial) | Form I |
| Form II | 2 h at 40° C. vacuum drying | Form V |
| | (open vial) | |
| Form VI | 1 h at 120° C. (closed vial) | amorphous |

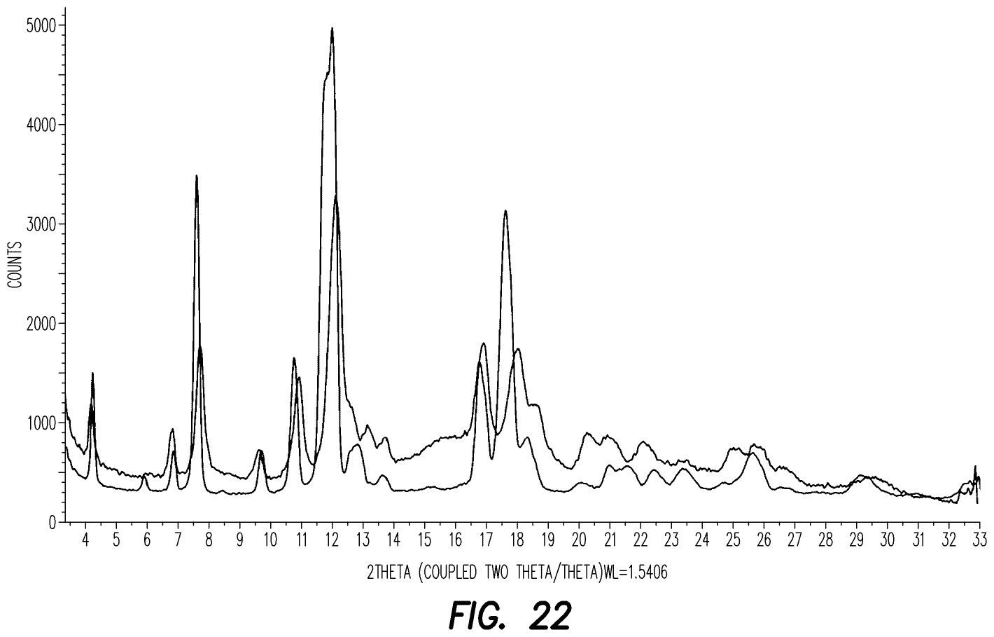

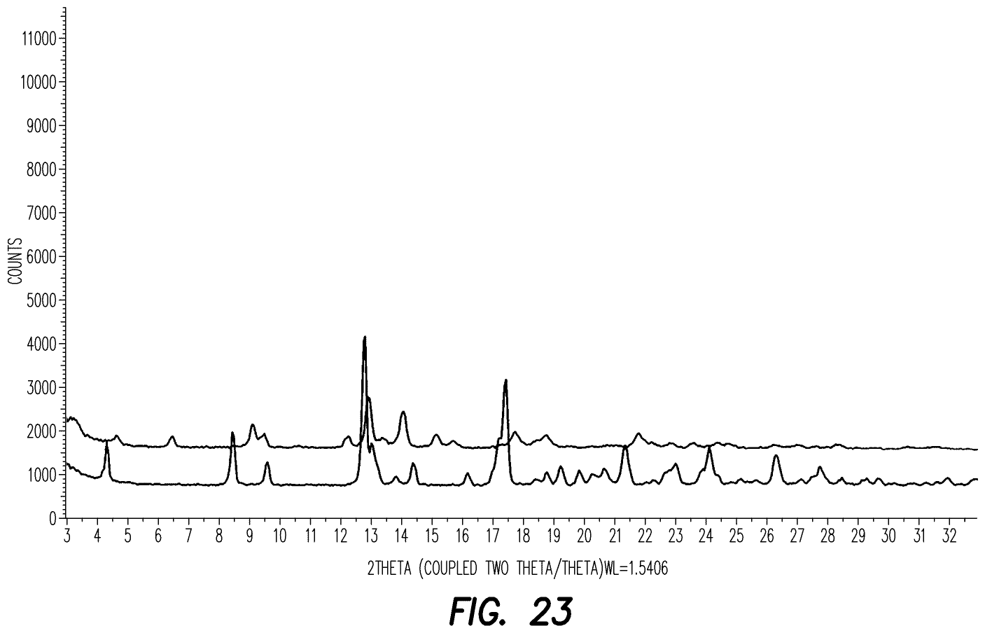

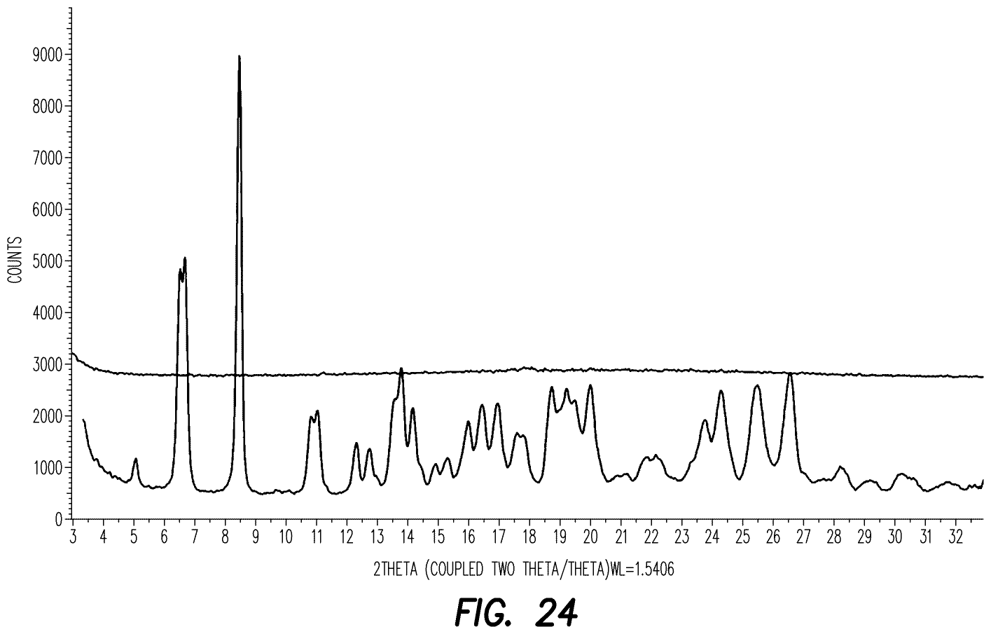

As can be seen from Table 14, although form I was exposed to the harshest temperature stress, it was the only form which remained unchanged (see also FIG. 22), while form II underwent a phase change to form V (see also FIG. 23) and form VI turned into amorphous compound (1) (see also FIG. 24). Reference XRPD diffractograms for forms I and V among others are shown in FIG. 25.

### Example 5—Chemical Stability of Solid State Forms of Compound (1)

To study form III and form IV under harsh stress conditions both crystalline forms were exposed to a range of elevated temperature (between 60° C. and 90° C.) and humidity (between 3% RH and 79% RH) over a duration of up to 21 days. To do so, control samples are stored refrigerated in the presence of desiccant for the duration of the study and measured simultaneously with stressed samples by HPLC at a wavelength of 252 nm.

The related substance results for form III and form IV are summarized in Table 15 and Table 16, respectively. No color changes were observed after stressing for either form III or form IV.

TABLE 15
Summary of the results for stressed form III samples

| | | Impurity | | | | | | | | |
|---|---|---|---|---|---|---|---|---|---|---|
| T [° C.] | % RH | A | B | C | D | E | F | G | H | I |
| Control 1 | | 0.059 | 0.012 | 0.026 | 0.127 | 0.049 | 0.008 | 0.016 | 0.139 | 0.014 |
| Control 2 | | 0.051 | 0.010 | 0.023 | 0.097 | 0.057 | 0.006 | 0.019 | 0.136 | 0.016 |
| Control 3 | | 0.069 | 0.010 | 0.024 | 0.143 | 0.051 | 0.007 | 0.019 | 0.139 | 0.016 |
| *60 | 75 | 0.067 | 0.012 | 0.023 | 0.134 | 0.057 | 0 | 0.019 | 0.140 | 0.014 |
| *70 | 62 | 0.064 | 0.012 | 0.024 | 0.119 | 0.059 | 0.006 | 0.019 | 0.139 | 0.015 |
| *75 | 41 | 0.066 | 0.011 | 0.025 | 0.118 | 0.062 | 0.009 | 0.019 | 0.136 | 0.010 |
| **75 | 79 | 0.066 | 0.012 | 0.031 | 0.106 | 0.074 | 0 | 0.005 | 0.135 | 0.015 |
| *80 | 11 | 0.066 | 0.011 | 0.025 | 0.123 | 0.054 | 0.007 | 0.024 | 0.141 | 0.015 |
| **80 | 61 | 0.052 | 0.013 | 0.024 | 0.096 | 0.060 | 0.009 | 0.020 | 0.137 | 0.015 |
| *90 | 3 | 0.064 | 0.012 | 0.033 | 0.087 | 0.073 | 0 | 0.007 | 0.140 | 0.016 |
| ***90 | 41 | 0.066 | 0.013 | 0.025 | 0.109 | 0.060 | 0 | 0.022 | 0.138 | 0.013 |
| *90 | 78 | 0.065 | 0.011 | 0.023 | 0.112 | 0.057 | 0.006 | 0.021 | 0.133 | 0.014 |

*samples measured after 21 days;
**samples measured after 17 days;
***sample measured after 10 days

For form III there was no significant increase in any related substance at any of the accelerated stress conditions. The lack of degradant formation with extreme stress (up to 21 days at 90° C./78% RH) confirms that form III is chemically very stable in the solid state.

TABLE 16
Summary of the results for stressed form IV samples

| | | Impurity | | | | | | | | |
|---|---|---|---|---|---|---|---|---|---|---|
| T [° C.] | % RH | A | B | C | D | E | F | G | H | I |
| Control 1 | | 0.102 | 0.009 | 0.064 | 0.011 | 0.074 | 0.015 | 0.037 | 0.129 | 0.012 |
| Control 2 | | 0.091 | 0.009 | 0.064 | 0.012 | 0.070 | 0.013 | 0.032 | 0.130 | 0.012 |
| Control 3 | | 0.114 | 0.009 | 0.070 | 0.013 | 0.071 | 0 | 0.023 | 0.129 | 0.012 |
| **60 | 75 | 0.111 | 0.008 | 0.066 | 0.014 | 0.074 | 0.012 | 0.037 | 0.132 | 0.012 |
| **70 | 62 | 0.113 | 0.010 | 0.065 | 0.022 | 0.069 | 0.013 | 0.038 | 0.133 | 0.012 |
| **75 | 41 | 0.113 | 0.010 | 0.072 | 0.015 | 0.078 | 0 | 0.027 | 0.129 | 0.010 |
| ***75 | 79 | 0.103 | 0.010 | 0.065 | 0.017 | 0.073 | 0.016 | 0.038 | 0.130 | 0.012 |
| **80 | 11 | 0.109 | 0.009 | 0.073 | 0.013 | 0.086 | 0 | 0.033 | 0.126 | 0.011 |
| ***80 | 61 | 0.111 | 0.008 | 0.065 | 0.021 | 0.070 | 0.015 | 0.039 | 0.131 | 0.011 |
| **90 | 3 | 0.104 | 0.011 | 0.070 | 0.018 | 0.087 | 0.008 | 0.050 | 0.125 | 0.011 |
| ***90 | 41 | 0.112 | 0.010 | 0.065 | 0.021 | 0.076 | 0.011 | 0.044 | 0.127 | 0.012 |
| **90 | 78 | 0.098 | 0.011 | 0.068 | 0.015 | 0.080 | 0.014 | 0.043 | 0.125 | 0.010 |

**samples measured after 17 days;
***samples measured after 10 days

Like form III, form IV shows no significant increase in any related substance at any of the accelerated stress conditions. The lack of degradant formation with extreme stress (up to 21 days at 90° C./78% RH) confirms that also form IV is chemically very stable in the solid state.

### Example 6—Manufacture of Solid Dispersions Comprising Compound (1) and Different Dispersion Polymers

In this Example, solid dispersions were prepared containing 25 wt % or 50 wt % compound (1) and 75 wt % or 50 wt %, respectively, of dispersion carriers HPMCAS-M (Shin-Etsu AQOAT), PVP-VA, Eudragit® L100 or HPMC HME 15LV.

The solid dispersions of this Example can be prepared according to the following protocol: the solid dispersion is spray dried from a spray solution composition comprising compound (1) as crystalline form I, III, IV (as described herein in the broadest form or in any embodiment thereof) or a mixture thereof, the dispersion carrier and a DCM:MeOH (1:1 (w/w)) solvent system with a solids content of 8 wt % total solids. Solid dispersions are manufactured using a Procept 4M8TRX spray drier with 2-fluid nozzle type and 1.0 mm/1.0 mm nozzle cap/tip dimension, an inlet temperature of 85-90° C., an outlet temperature of 45-50° C., atomization at 3.0 bar, drying gas air flow rate of 0.50 m3/min, and a solution feed rate of approx. 15 g/min. Secondary drying of the dispersion is done in a vacuum dryer of tray dryer type in collection vessels, at 40° C. for 22.5 hours.

The spray drying yield results obtained following the protocol from the previous paragraph are summarized in Table 17, where gA stands for gram API (active pharmaceutical ingredient, i.e. compound (1)).

TABLE 17
Batch sizes and yield for solid dispersions of compound (1) with dispersion
carriers HPMCAS-M, PVP-VA, Eudragit ® L100 or HPMC HME 15LV

| | Batch size | | Yield | |
|---|---|---|---|---|
| Formulation | [gA] | | [%]/gA | |
| 25 wt %:75 wt % compound (1):HPMCAS-M | 12 | gA | 85% | (10.2 g) |
| 50 wt %:50 wt % compound (1):HPMCAS-M | 0.25 | gA | 64% | (0.16 gA) |
| 25 wt %:75 wt % compound (1):PVP-VA | 12 | gA | 79% | (9.5 gA) |
| 50 wt %:50 wt % compound (1):PVP-VA | 0.25 | gA | 72% | (0.18 gA) |
| 25 wt %:75 wt % compound (1):Eudragit ® L100 | 0.25 | gA | 76% | (0.19 gA) |
| 50 wt %:50 wt % compound (1):Eudragit ® L100 | 0.25 | gA | 60% | (0.15 gA) |
| 25 wt %:75 wt % compound (1):HPMC HME 15LV | 0.25 | gA | 52% | (0.13 g) |
| 50 wt %:50 wt % compound (1):HPMC HME 15LV | 0.25 | gA | 32% | (0.08 gA) |

### Example 7—Characterization of Solid Dispersions by X-Ray Powder Diffraction (XRPD)

XRPDs can be obtained according to the following protocol: XRPD analysis is done with a Rigaku Miniflexx 600 diffractometer. An amount of approx. 10 mg of samples of the solid dispersions of compound (1) with dispersion carriers, e.g. with HPMCAS-M, PVP-VA, Eudragit® L100 or HPMC HME 15LV as in Example 6, is placed onto a zero-background sample disk and placed into the auto sampler of the Rigaku Miniflex 600. Samples are analyzed using the instrument parameters described in Table 18 below.

TABLE 18
Summary of XRPD collection parameters

| Parameter | Value | |
|---|---|---|
| Θ/2-Θ | 3-60° | |
| Radiation source | Cu—Kα1,2 (1.54184 Å) | |
| Spin | On | |
| Step size | 0.02° | |
| Step rate | 2.0 deg/min | |
| Voltage | 40 | kV |
| Current | 15 | mA |
| Divergent slit | 1.0 | mm |

| | |
|---|---|
| Holder | Zero-Background |

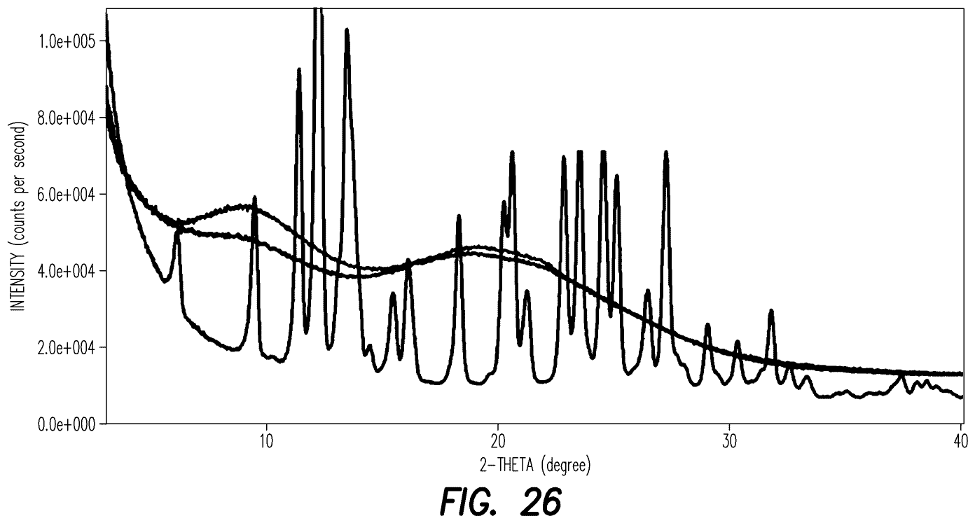

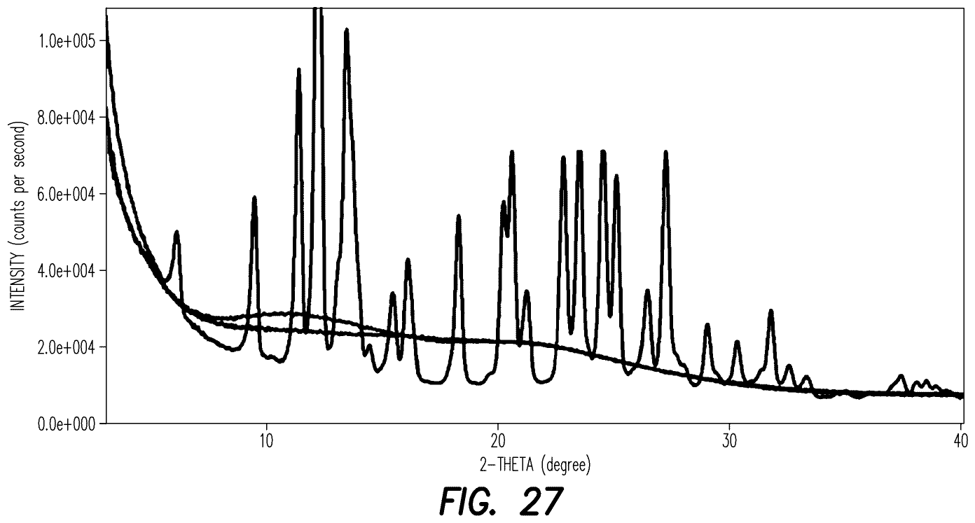

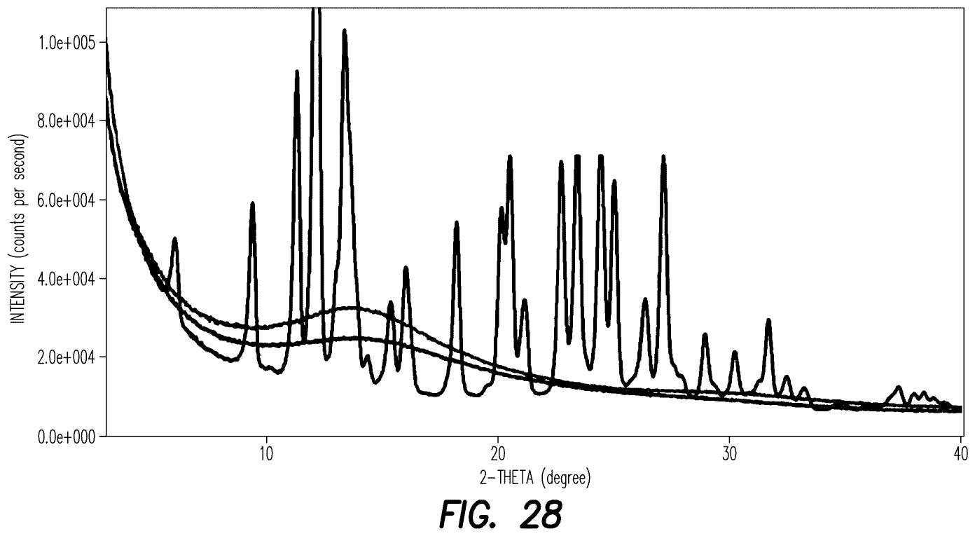

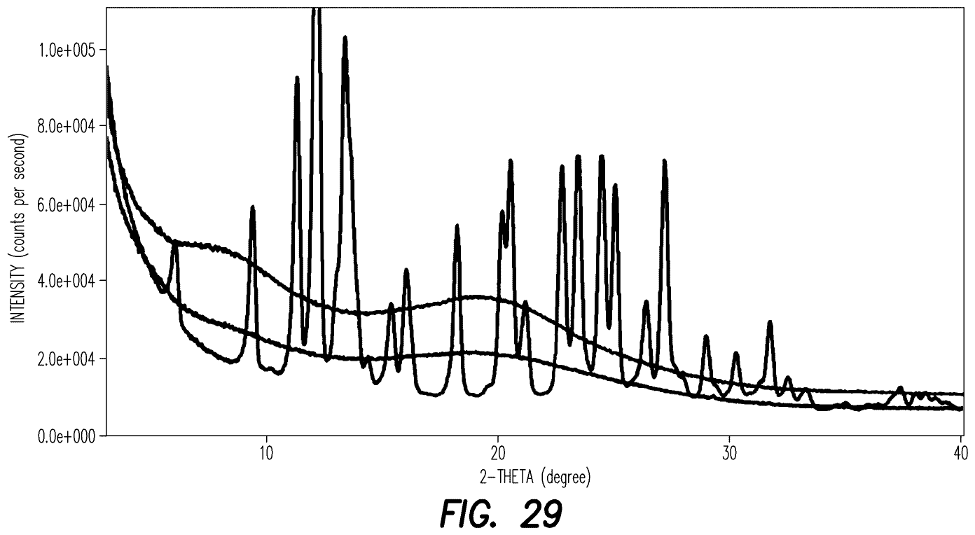

The XRPDs obtained following the protocol from the previous paragraph with the various solid dispersions prepared under Example 6 are displayed in FIGS. 26 to 29. Comparing them against the XRPD of crystalline compound (1) in the same Figures indicates the absence of crystalline material in the samples. Specifically, the XRPDs of the solid dispersion of 25 wt % or 50 wt % compound (1) and dispersion carriers HPMCAS-M, PVP-VA, Eudragit® L100 or

HPMC HME 15LV exhibited a lack of sharp peaks and the presence of amorphous halos. The lack of sharp diffraction peaks is indicative that the solid dispersions are consistent with an amorphous form of compound (1).

1. A crystalline form of compound (1) 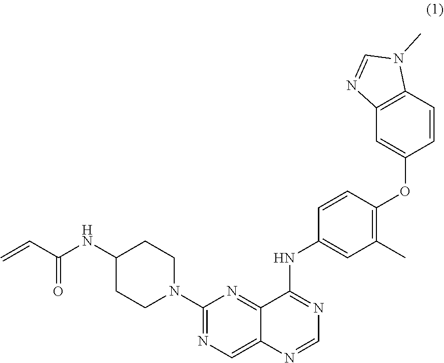 [SMILES: C=CC(=O)NC1CCN(c2ncc3ncnc(Nc4ccc(Oc5ccc6c(c5)ncn6C)c(C)c4)c3n2)CC1]
characterized by having:
(a) a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°) and (16.7±0.2°);
and/or
(b) a Raman spectrum comprising a peak at any one of the following wavenumbers expressed as inverse centimeters when measured at a temperature in the range of from 20 to 30° C. and a wavelength of 785 nm: 640±2 and/or 831±2.

2. The crystalline form according to claim 1, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°) and (16.7±0.2°).

3. The crystalline form according to claim 1 or 2, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (5.9±0.2°), (11.7±0.2°), (14.7±0.2°), (16.7±0.2°), (18.8 ±0.2°) and (19.2±0.2°).

4. The crystalline form according to any one of claims 1 to 3, characterized by having a Raman spectrum comprising a peak at any one of the following wavenumbers expressed as inverse centimeters when measured at a temperature in the range of from 20 to 30° C. and a wavelength of 785 nm: 640±2 and/or 831±2.

5. The crystalline form according to any one of claims 1 to 4, characterized by having a Raman spectrum comprising peaks at the following wavenumbers expressed as inverse centimeters when measured at a temperature in the range of from 20 to 30° C. and a wavelength of 785 nm: 238±2, 640±2 and 831±2.

6. A crystalline form of compound (1) 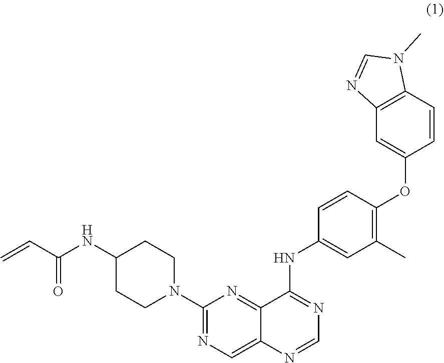 [SMILES: C=CC(=O)NC1CCN(c2ncc3ncnc(Nc4ccc(Oc5ccc6c(c5)ncn6C)c(C)c4)c3n2)CC1]
characterized by having:
(a) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4 +0.2°) and (16.2±0.2°); or
(b) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°); or
(c) a Raman spectrum comprising a peak at any one of the following wavenumbers expressed as inverse centimeters when measured at a temperature in the range of from 20 to 30° C. and a wavelength of 785 nm: 1310±2 and/or 1400±2; or
(d) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°) and (16.2±0.2°) and a Raman spectrum comprising a peak at any one of the following wavenumbers expressed as inverse centimeters when measured at a temperature in the range of from 20 to 30° C. and a wavelength of 785 nm: 1310±2 and/or 1400±2; or
(e) a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°) and a Raman spectrum comprising a peak at any one of the following wavenumbers expressed as inverse centimeters when measured at a temperature in the range of from 20 to 30° C. and a wavelength of 785 nm: 1310±2 and/or 1400±2.

7. The crystalline form according to claim 6, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°) and (16.2±0.2°).

8. The crystalline form according to claim 6 or 7, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4±0.2°), (16.2 ±0.2°) and (18.3±0.2°).

9. The crystalline form according to any one of claims 6 to 8, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°).

10. The crystalline form according to any one of claims 6 to 9, characterized by having a Raman spectrum comprising a peak at any one of the following wavenumbers expressed as inverse centimeters when measured at a temperature in the range of from 20 to 30° C. and a wavelength of 785 nm: 1310±2 and/or 1400±2.

11. The crystalline form according to any one of claims 6 to 10, characterized by having a Raman spectrum comprising peaks at the following wavenumbers expressed as inverse centimeters when measured at a temperature in the range of from 20 to 30° C. and a wavelength of 785 nm: 485±2, 610±2, 1029±2, 1310±2 and 1400±2.

12. The crystalline form according to any one of claims 6 to 11, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.2°), (9.5±0.2°), (11.4±0.2°), (12.4 +0.2°) and (16.2±0.2°) and a Raman spectrum comprising a peak at any one of the following wavenumbers expressed as inverse centimeters when measured at a temperature in the range of from 20 to 30° C. and a wavelength of 785 nm: 1310±2 and/or 1400±2.

13. The crystalline form according to any one of claims 6 to 12, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (6.2±0.1°), (9.5±0.1°), (11.4±0.1°) and (12.4±0.1°) and a Raman spectrum comprising a peak at any one of the following wavenumbers expressed as inverse centimeters when measured at a temperature in the range of from 20 to 30° C. and a wavelength of 785 nm: 1310±2 and/or 1400±2.

14. A crystalline form of compound (1) 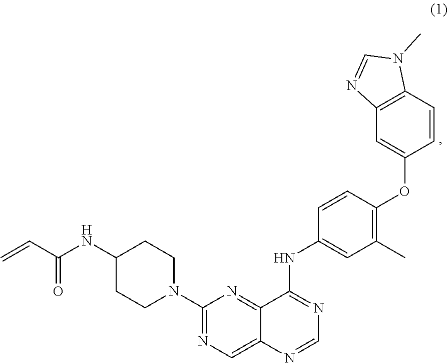 [SMILES: C=CC(=O)NC1CCN(c2ncc3ncnc(Nc4ccc(Oc5ccc6c(c5)ncn6C)c(C)c4)c3n2)CC1]
characterized by having:
(a) a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.0±0.2°); and/or
(b) a Raman spectrum comprising a peak at any one of the following wavenumbers expressed as inverse centimeters when measured at a temperature in the range of from 20 to 30° C. and a wavelength of 785 nm: 1411±2 and/or 1602±2.

15. The crystalline form according to claim 14, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (7.9±0.2°) and (12.0±0.2°).

16. The crystalline form according to claim 14 or 15, characterized by having a powder X-ray diffraction pattern comprising peaks at the following 2θ values when measured at a temperature in the range of from 20 to 30° C. using CuK_{α} radiation having a wavelength of 1.54056 Å or 1.54184 Å: (6.1±0.2°), (7.9±0.2°), (11.1±0.2°), (12.0±0.2°), (17.2 ±0.2°) and (17.9±0.2°).

17. The crystalline form according to any one of claims 14 to 16, characterized by having a Raman spectrum comprising a peak at any one of the following wavenumbers expressed as inverse centimeters when measured at a temperature in the range of from 20 to 30° C. and a wavelength of 785 nm: 1411±2 and/or 1602±2.

18. The crystalline form according to any one of claims 14 to 17, characterized by having a Raman spectrum comprising peaks at the following wavenumbers expressed as inverse centimeters when measured at a temperature in the range of from 20 to 30° C. and a wavelength of 785 nm: 824±2, 1411±2 and 1602±2.

19. A process for the preparation of a crystalline form of compound (1): 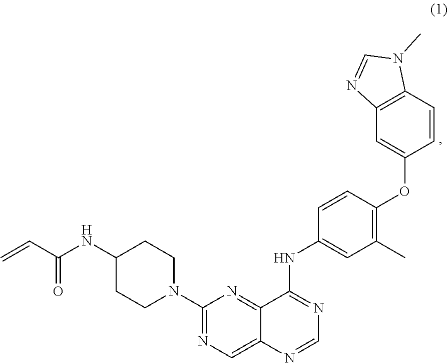 [SMILES: C=CC(=O)NC1CCN(c2ncc3ncnc(Nc4ccc(Oc5ccc6c(c5)ncn6C)c(C)c4)c3n2)CC1]
comprising the following steps:
i) dissolving compound (1) upon heating in at least one organic solvent;
ii) cooling the solution obtained in step (i), wherein a crystalline form of compound (1) is formed; and
iii) isolating at least a part of the crystalline form of compound (1) obtained in step (ii).

20. The process according to claim 19, wherein the crystalline form of compound (1) as defined in any one of claims 1 to 5 is at least in part formed.

21. The crystalline form obtainable or obtained by the process according to claim 19 or 20.

22. The crystalline form according to claim 21, characterized by having a powder X-ray diffraction pattern comprising the peaks as defined in any one of claims 1 to 5 and/or a Raman spectrum comprising peaks at the wavenumbers as defined in any one of claims 1 to 5.

23. A process for the preparation of a crystalline form of compound (1): 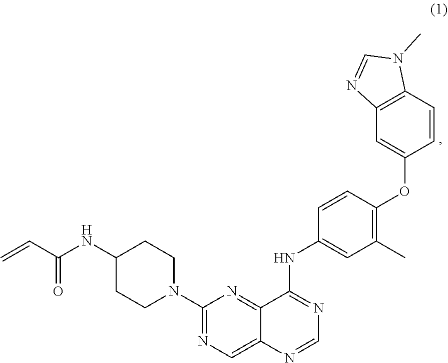 [SMILES: C=CC(=O)NC1CCN(c2ncc3ncnc(Nc4ccc(Oc5ccc6c(c5)ncn6C)c(C)c4)c3n2)CC1]
comprising the following steps:
i) providing a suspension of compound (1) in at least one organic solvent;
ii) agitating the suspension obtained in step (i) until a crystalline form of compound (1) is formed; and
iii) isolating at least a part of the crystalline form of compound (1) obtained in step (ii).

24. The process according to claim 23, wherein the crystalline form of compound (1) as defined in any one of claims 6 to 13 is at least in part formed.

25. The crystalline form obtainable or obtained by the process according to claim 23 or 24.

26. The crystalline form according to claim 25, characterized by having a powder X-ray diffraction pattern comprising the peaks as defined in any one of claims 6 to 13 and/or a Raman spectrum comprising peaks at the wavenumbers as defined in any one of claims 6 to 13.

27. A process for the preparation of a crystalline form of compound (1): 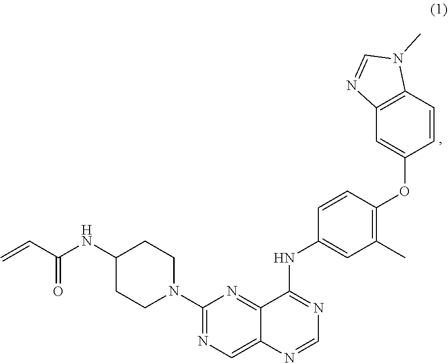 [SMILES: C=CC(=O)NC1CCN(c2ncc3ncnc(Nc4ccc(Oc5ccc6c(c5)ncn6C)c(C)c4)c3n2)CC1]
comprising the following steps:
i) dissolving compound (1) upon heating in a mixture of solvents comprising at least one water-miscible organic solvent and water;
ii) cooling the solution obtained in step (i), wherein a crystalline form of compound (1) is formed; and
iii) isolating at least a part of the crystalline form of compound (1) obtained in step (ii).

28. The process according to claim 27, wherein the crystalline form of compound (1) as defined in any one of claims 14 to 18 is at least in part formed.

29. A crystalline form obtainable or obtained by the process according to any one of claim 27 or 28.

30. The crystalline form according to claim 29, characterized by having a powder X-ray diffraction pattern comprising the peaks as defined in any one of claims 14 to 18 and/or a Raman spectrum comprising peaks at the wavenumbers as defined in any one of claims 14 to 18.

31. The crystalline form as defined in any one of claim 1 to 18, 21, 22, 25, 26, 29 or 30, for use as a medicament.

32. The crystalline form as defined in any one of claim 1 to 18, 21, 22, 25, 26, 29 or 30, for use in the treatment and/or prevention of an oncological and/or hyperproliferative disease, in particular cancer.

33. The crystalline form for use according to claim 32, wherein the oncological and/or hyperproliferative disease is a cancer selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

34. The crystalline form for use according to claim 32 or 33, wherein the oncological and/or hyperproliferative disease is a HER2 overexpressed, HER2 amplified and/or HER2 mutant cancer.

35. A pharmaceutical composition comprising the crystalline form as defined in any one of claim 1 to 18, 21, 22, 25, 26, 29 or 30 and one or more pharmaceutically acceptable excipients.

36. Use of the crystalline form as defined in any one of claim 1 to 18, 21, 22, 25, 26, 29 or 30 or of a mixture thereof to prepare a solid dispersion comprising compound (1) and a pharmaceutically acceptable dispersion carrier.

37. Use according to claim 36, wherein the solid dispersion comprises compound (1) in amorphous form.

38. A process of preparing a solid dispersion comprising the steps of:
a) providing a mixture of compound (1) and a pharmaceutically acceptable dispersion carrier and adding a solvent to obtain a solution or a suspension; and
b) removing the solvent from the solution or the suspension to form the solid dispersion, wherein
in step a), compound (1) is provided as the crystalline form as defined in any one of claim 1 to 18, 21, 22, 25, 26, 29 or 30 or as a mixture thereof.

39. The process according to claim 38, wherein in step b), the solid dispersion comprises compound (1) in amorphous form.
